SEQUENCE LISTING (650 sequences)

SEQ ID NO: 1 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 2 - protein, 7 aa, synthetic construct
  VAR_SEQ 1: note = Xaa isselected from L, G, S, A, Q, and D
  VAR_SEQ 6: note = Xaa is selected from S, P, Q, and N
  VAR_SEQ 7: note = Xaa is selected from V, A, S, I, and F
XNTTKXX

SEQ ID NO: 3 - protein, 11 aa, synthetic construct
AQSNTTKSVDQ

SEQ ID NO: 4 - protein, 11 aa, synthetic construct
AQQNTTKSVDQ

SEQ ID NO: 5 - protein, 11 aa, synthetic construct
ADLNTTKPVMQ

SEQ ID NO: 6 - protein, 11 aa, synthetic construct
AQANTTKSVDQ

SEQ ID NO: 7 - protein, 11 aa, synthetic construct
AQNNTTKSVDQ

SEQ ID NO: 8 - protein, 11 aa, synthetic construct
AQLNTTKNVDQ

SEQ ID NO: 9 - protein, 11 aa, synthetic construct
ADLNTTKSVAQ

SEQ ID NO: 10 - protein, 11 aa, synthetic construct
AQDNTTKSVFQ

SEQ ID NO: 11 - protein, 11 aa, synthetic construct
ADLNTTKSVSQ

SEQ ID NO: 12 - protein, 11 aa, synthetic construct
AQGNTTKSVDQ

SEQ ID NO: 13 - protein, 11 aa, synthetic construct
AQGNTTKSVQQ

SEQ ID NO: 14 - protein, 11 aa, synthetic construct
AQLNTTKSVDQ

SEQ ID NO: 15 - protein, 11 aa, synthetic construct
ADLNTTKSVNQ

SEQ ID NO: 16 - protein, 11 aa, synthetic construct
AQSNTTKQVMQ

SEQ ID NO: 17 - protein, 11 aa, synthetic construct
AQHNTTKSVDQ

SEQ ID NO: 18 - protein, 11 aa, synthetic construct
AQGNTTKSSMQ

SEQ ID NO: 19 - protein, 11 aa, synthetic construct
ADLNTTKNVMQ

SEQ ID NO: 20 - protein, 11 aa, synthetic construct
ADLNTTKSVFQ

SEQ ID NO: 21 - protein, 11 aa, synthetic construct
AQLNTTKSVMQ

SEQ ID NO: 22 - protein, 11 aa, synthetic construct
ADLNTTKSIMQ

SEQ ID NO: 23 - protein, 11 aa, synthetic construct
AILNTTKSVDQ

SEQ ID NO: 24 - protein, 11 aa, synthetic construct
PELNTTKSVMQ

SEQ ID NO: 25 - protein, 11 aa, synthetic construct
AQLNTTKSIDQ

SEQ ID NO: 26 - protein, 11 aa, synthetic construct
AQLNTTKIVDQ

SEQ ID NO: 27 - protein, 11 aa, synthetic construct
AQLNTTKSVDG

SEQ ID NO: 28 - protein, 11 aa, synthetic construct
AQLNTTKQVDQ

SEQ ID NO: 29 - protein, 11 aa, synthetic construct
AHLNTTKSVDQ

SEQ ID NO: 30 - protein, 11 aa, synthetic construct
ADLNTTKQVMQ

SEQ ID NO: 31 - protein, 11 aa, synthetic construct
AQGNTTKSAMQ

SEQ ID NO: 32 - protein, 11 aa, synthetic construct
ANGNTTKSVMQ

SEQ ID NO: 33 - protein, 11 aa, synthetic construct
AQDNTTKSVQQ

SEQ ID NO: 34 - protein, 11 aa, synthetic construct
AQSNTTKAVMQ

SEQ ID NO: 35 - protein, 11 aa, synthetic construct
AQQNTTKSVQQ

SEQ ID NO: 36 - protein, 11 aa, synthetic construct
ADLNTTKSVGQ

SEQ ID NO: 37 - protein, 11 aa, synthetic construct
DQQNTTKSVMQ

SEQ ID NO: 38 - protein, 11 aa, synthetic construct
AQLNTTKSFDQ

SEQ ID NO: 39 - protein, 11 aa, synthetic construct
AQSNTTKSVMG

SEQ ID NO: 40 - protein, 11 aa, synthetic construct
AQANTTKSVNQ

SEQ ID NO: 41 - protein, 11 aa, synthetic construct
AQANTTKQVMQ

SEQ ID NO: 42 - protein, 11 aa, synthetic construct
AQGNTTKSVMQ

SEQ ID NO: 43 - protein, 11 aa, synthetic construct
PQLNTTKSAMQ

SEQ ID NO: 44 - protein, 11 aa, synthetic construct
AALNTTKSVDQ

SEQ ID NO: 45 - protein, 11 aa, synthetic construct
APLNTTKSVDQ

SEQ ID NO: 46 - protein, 11 aa, synthetic construct
AQSNTTKSVNQ

SEQ ID NO: 47 - protein, 11 aa, synthetic construct
AQLNTTKAVDQ

SEQ ID NO: 48 - protein, 11 aa, synthetic construct
DQLNTTKSVNQ

SEQ ID NO: 49 - protein, 11 aa, synthetic construct
AQQNTTKSVMQ

SEQ ID NO: 50 - protein, 11 aa, synthetic construct
AQLNTTKSVDS

SEQ ID NO: 51 - protein, 11 aa, synthetic construct
AQDNTTKSVSQ

SEQ ID NO: 52 - protein, 11 aa, synthetic construct
AQGNTTKSVAQ

SEQ ID NO: 53 - protein, 11 aa, synthetic construct
AQQNTTKSVNQ

SEQ ID NO: 54 - protein, 11 aa, synthetic construct
AQLNTTKSVDN

SEQ ID NO: 55 - protein, 11 aa, synthetic construct
AQANTTKSVQQ

SEQ ID NO: 56 - protein, 11 aa, synthetic construct
AQLNTTKHVDQ

SEQ ID NO: 57 - protein, 11 aa, synthetic construct
AQLNTTKSHDQ

SEQ ID NO: 58 - protein, 11 aa, synthetic construct
DQANTTKSVMQ

SEQ ID NO: 59 - protein, 11 aa, synthetic construct
AQGNTTKSVMP

SEQ ID NO: 60 - protein, 11 aa, synthetic construct
PQLNTTKSVDQ

SEQ ID NO: 61 - protein, 11 aa, synthetic construct
AQDNTTKSVNQ

SEQ ID NO: 62 - protein, 11 aa, synthetic construct
AQLNTTKSVQN

SEQ ID NO: 63 - protein, 11 aa, synthetic construct
AQLNTTKSVAG

SEQ ID NO: 64 - protein, 11 aa, synthetic construct
AQLNTTKIVSQ

SEQ ID NO: 65 - protein, 11 aa, synthetic construct
PQLNTTKGVMQ

SEQ ID NO: 66 - protein, 11 aa, synthetic construct
AQSNTTKSVMQ

SEQ ID NO: 67 - protein, 11 aa, synthetic construct
PQLNTTKSQMQ

SEQ ID NO: 68 - protein, 11 aa, synthetic construct
AQLNTTKSVNQ

SEQ ID NO: 69 - protein, 11 aa, synthetic construct
AQLNTTKSVNG

SEQ ID NO: 70 - protein, 11 aa, synthetic construct
EQLNTTKSVMG

SEQ ID NO: 71 - protein, 11 aa, synthetic construct
AQLNTTKSVDA

SEQ ID NO: 72 - protein, 11 aa, synthetic construct
AQQNTTKSVMG

SEQ ID NO: 73 - protein, 11 aa, synthetic construct
PQLNTTKSPMQ

SEQ ID NO: 74 - protein, 11 aa, synthetic construct
AQLNTTKQVNQ

SEQ ID NO: 75 - protein, 11 aa, synthetic construct
ASLNTTKPVMQ

SEQ ID NO: 76 - protein, 11 aa, synthetic construct
AQLNTTKSVMG

SEQ ID NO: 77 - protein, 11 aa, synthetic construct
AQLNTTKSVQQ

SEQ ID NO: 78 - protein, 11 aa, synthetic construct
AQLNQTKSVDQ

SEQ ID NO: 79 - protein, 11 aa, synthetic construct
PQSNTTKSVMQ

SEQ ID NO: 80 - protein, 11 aa, synthetic construct
ADLNTTKSVMQ

SEQ ID NO: 81 - protein, 11 aa, synthetic construct
AQINTTKSVDQ

SEQ ID NO: 82 - protein, 11 aa, synthetic construct
AQLNTTKSVDH

SEQ ID NO: 83 - protein, 11 aa, synthetic construct
EQLNTTKSVMQ

SEQ ID NO: 84 - protein, 11 aa, synthetic construct
PQLNTTKSSMQ

SEQ ID NO: 85 - protein, 11 aa, synthetic construct
AQLNTTKAVFQ

SEQ ID NO: 86 - protein, 11 aa, synthetic construct
SQGNTTKSVMQ

SEQ ID NO: 87 - protein, 11 aa, synthetic construct
AQLNTTKAVNQ

SEQ ID NO: 88 - protein, 11 aa, synthetic construct
AQLNTTKSVDI

SEQ ID NO: 89 - protein, 11 aa, synthetic construct
AQANTTKSVMG

SEQ ID NO: 90 - protein, 11 aa, synthetic construct
AQLNTTKQVSQ

SEQ ID NO: 91 - protein, 11 aa, synthetic construct
AQNNTTKSVNQ

SEQ ID NO: 92 - protein, 11 aa, synthetic construct
AQGNTTKSQMQ

SEQ ID NO: 93 - protein, 11 aa, synthetic construct
PQLNTTKSVAQ

SEQ ID NO: 94 - protein, 11 aa, synthetic construct
ADLNTTKSVQQ

SEQ ID NO: 95 - protein, 11 aa, synthetic construct
AQQNTTKSVMA

SEQ ID NO: 96 - protein, 11 aa, synthetic construct
DQLNTTKSSMQ

SEQ ID NO: 97 - protein, 11 aa, synthetic construct
AQENTVRNMAQ

SEQ ID NO: 98 - protein, 11 aa, synthetic construct
AQQNTTKIVMQ

SEQ ID NO: 99 - protein, 11 aa, synthetic construct
AHDNTTKSVMQ

SEQ ID NO: 100 - protein, 11 aa, synthetic construct
AQANTTKIVMQ

SEQ ID NO: 101 - protein, 11 aa, synthetic construct
AQGNTTKQVMQ

SEQ ID NO: 102 - protein, 11 aa, synthetic construct
AQLNTTKQVMI

SEQ ID NO: 103 - protein, 11 aa, synthetic construct
AQANTTKAVMQ

SEQ ID NO: 104 - protein, 11 aa, synthetic construct
AQDNTTKSVMQ

SEQ ID NO: 105 - protein, 11 aa, synthetic construct
AQNNTTKQVMQ

SEQ ID NO: 106 - protein, 11 aa, synthetic construct
QQLNTTKSVDQ

SEQ ID NO: 107 - protein, 11 aa, synthetic construct
AQDNTTKSVAQ

SEQ ID NO: 108 - protein, 11 aa, synthetic construct
AQGNTTKSNMQ

SEQ ID NO: 109 - protein, 11 aa, synthetic construct
ADLNTTKIVMQ

SEQ ID NO: 110 - protein, 11 aa, synthetic construct
AQDNTTKQVMQ

SEQ ID NO: 111 - protein, 11 aa, synthetic construct
AQSNTTKSVSQ

SEQ ID NO: 112 - protein, 11 aa, synthetic construct
AQSNTTKSVQQ

SEQ ID NO: 113 - protein, 11 aa, synthetic construct
PQLNTTKSVGQ

SEQ ID NO: 114 - protein, 11 aa, synthetic construct
PQLNTTKSGMQ

SEQ ID NO: 115 - protein, 11 aa, synthetic construct
AQLNTTKNVAQ

SEQ ID NO: 116 - protein, 11 aa, synthetic construct
AQLNTTKIVGQ

SEQ ID NO: 117 - protein, 11 aa, synthetic construct
DQGNTTKSVMQ

SEQ ID NO: 118 - protein, 11 aa, synthetic construct
AQLNTTKSVQG

SEQ ID NO: 119 - protein, 11 aa, synthetic construct
IQLNTTKSVDQ

SEQ ID NO: 120 - protein, 11 aa, synthetic construct
AQDNTTKSVHQ

SEQ ID NO: 121 - protein, 11 aa, synthetic construct
AQLNHTKSVDQ

SEQ ID NO: 122 - protein, 11 aa, synthetic construct
AQLNTTKAVGQ

SEQ ID NO: 123 - protein, 11 aa, synthetic construct
AQNNTTKSVFQ

SEQ ID NO: 124 - protein, 11 aa, synthetic construct
AQQNTTKAVMQ

SEQ ID NO: 125 - protein, 11 aa, synthetic construct
ADLNTTKSVMG

SEQ ID NO: 126 - protein, 11 aa, synthetic construct
AALNTTKPVMQ

SEQ ID NO: 127 - protein, 11 aa, synthetic construct
PQANTTKSVMQ

SEQ ID NO: 128 - protein, 11 aa, synthetic construct
AQLNTTKSVNN

SEQ ID NO: 129 - protein, 11 aa, synthetic construct
AQGNTTKAVMQ

SEQ ID NO: 130 - protein, 11 aa, synthetic construct
AQQNTTKQVMQ

SEQ ID NO: 131 - protein, 11 aa, synthetic construct
ADLNTTKSVHQ

SEQ ID NO: 132 - protein, 11 aa, synthetic construct
PQLNTTKSDMQ

SEQ ID NO: 133 - protein, 11 aa, synthetic construct
AQLNTTKQVMQ

SEQ ID NO: 134 - protein, 11 aa, synthetic construct
ADLNTTKAVMQ

SEQ ID NO: 135 - protein, 11 aa, synthetic construct
AQGNTTKDVMQ

SEQ ID NO: 136 - protein, 11 aa, synthetic construct
AQANTTKPVMQ

SEQ ID NO: 137 - protein, 11 aa, synthetic construct
AQLNTTKAVSQ

SEQ ID NO: 138 - protein, 11 aa, synthetic construct
AQSNTTKPVMQ

SEQ ID NO: 139 - protein, 11 aa, synthetic construct
AQLNTTKQVGQ

SEQ ID NO: 140 - protein, 11 aa, synthetic construct
AQGNTTKSVMA

SEQ ID NO: 141 - protein, 11 aa, synthetic construct
PQLNTTKSVSQ

SEQ ID NO: 142 - protein, 11 aa, synthetic construct
AQLNTTKNVPQ

SEQ ID NO: 143 - protein, 11 aa, synthetic construct
AQQNTTKSVAQ

SEQ ID NO: 144 - protein, 11 aa, synthetic construct
AQSNTTKSVFQ

SEQ ID NO: 145 - protein, 11 aa, synthetic construct
ESLNTTKSVMQ

SEQ ID NO: 146 - protein, 11 aa, synthetic construct
AQLNTTKSINQ

SEQ ID NO: 147 - protein, 11 aa, synthetic construct
ADQNTTKSVMQ

SEQ ID NO: 148 - protein, 11 aa, synthetic construct
DQLNTTKPVMQ

SEQ ID NO: 149 - protein, 11 aa, synthetic construct
AQLNTTKNVSQ

SEQ ID NO: 150 - protein, 11 aa, synthetic construct
AQNNTTKSVQQ

SEQ ID NO: 151 - protein, 11 aa, synthetic construct
AQQNTTKSIMQ

SEQ ID NO: 152 - protein, 11 aa, synthetic construct
AQLNTTKSVNP

SEQ ID NO: 153 - protein, 11 aa, synthetic construct
AQDNTTKAVMQ

SEQ ID NO: 154 - protein, 11 aa, synthetic construct
AQDNTTKSVDQ

SEQ ID NO: 155 - protein, 11 aa, synthetic construct
AAGNTTKSVMQ

SEQ ID NO: 156 - protein, 11 aa, synthetic construct
AQLNTTKSVDF

SEQ ID NO: 157 - protein, 11 aa, synthetic construct
AQLNTTKQVQQ

SEQ ID NO: 158 - protein, 11 aa, synthetic construct
AQLNTTKSQPQ

SEQ ID NO: 159 - protein, 11 aa, synthetic construct
AQGNTTKSIMQ

SEQ ID NO: 160 - protein, 11 aa, synthetic construct
QELNTTKSVMQ

SEQ ID NO: 161 - protein, 11 aa, synthetic construct
AQQNTTKSVFQ

SEQ ID NO: 162 - protein, 11 aa, synthetic construct
AMLNTTKSVMG

SEQ ID NO: 163 - protein, 11 aa, synthetic construct
AQDNTTKSHMQ

SEQ ID NO: 164 - protein, 11 aa, synthetic construct
NQGNTTKSVMQ

SEQ ID NO: 165 - protein, 11 aa, synthetic construct
AQLNTTKQVAQ

SEQ ID NO: 166 - protein, 11 aa, synthetic construct
AQSNTTKSVGQ

SEQ ID NO: 167 - protein, 11 aa, synthetic construct
AQDNTTKSFMQ

SEQ ID NO: 168 - protein, 11 aa, synthetic construct
ADLNTTKHVMQ

SEQ ID NO: 169 - protein, 11 aa, synthetic construct
AQLNTTKSVNS

SEQ ID NO: 170 - protein, 11 aa, synthetic construct
AQLNTTKNVMQ

SEQ ID NO: 171 - protein, 11 aa, synthetic construct
AQLNTTKAVQQ

SEQ ID NO: 172 - protein, 11 aa, synthetic construct
PDLNTTKSVMQ

SEQ ID NO: 173 - protein, 11 aa, synthetic construct
AQGNTTKSVSQ

SEQ ID NO: 174 - protein, 11 aa, synthetic construct
AQDNTTKPVMQ

SEQ ID NO: 175 - protein, 11 aa, synthetic construct
AQSNTTKSVMS

SEQ ID NO: 176 - protein, 11 aa, synthetic construct
AQYNSTKNMAQ

SEQ ID NO: 177 - protein, 11 aa, synthetic construct
AQSNTTKSVMA

SEQ ID NO: 178 - protein, 11 aa, synthetic construct
PQLNTTKSVMQ

SEQ ID NO: 179 - protein, 11 aa, synthetic construct
AQLNTTKQIMQ

SEQ ID NO: 180 - protein, 11 aa, synthetic construct
GQGNTTKSVMQ

SEQ ID NO: 181 - protein, 11 aa, synthetic construct
PELNTTKSVMS

SEQ ID NO: 182 - protein, 11 aa, synthetic construct
MDLNTTKSVMG

SEQ ID NO: 183 - protein, 11 aa, synthetic construct
AQLNTTKSVND

SEQ ID NO: 184 - protein, 11 aa, synthetic construct
AQLNTTKSVGQ

SEQ ID NO: 185 - protein, 11 aa, synthetic construct
AQANTTKSVMQ

SEQ ID NO: 186 - protein, 11 aa, synthetic construct
AQLNTTKIVMQ

SEQ ID NO: 187 - protein, 11 aa, synthetic construct
PQQNTTKSVMQ

SEQ ID NO: 188 - protein, 11 aa, synthetic construct
AQLNTTKSVFG

SEQ ID NO: 189 - protein, 11 aa, synthetic construct
AQLNTTKAVPQ

SEQ ID NO: 190 - protein, 11 aa, synthetic construct
AQQNTTKNVMQ

SEQ ID NO: 191 - protein, 11 aa, synthetic construct
AQGNAIRNGAQ

SEQ ID NO: 192 - protein, 11 aa, synthetic construct
AQLNTTKSANQ

SEQ ID NO: 193 - protein, 11 aa, synthetic construct
AQNNTTKSVMG

SEQ ID NO: 194 - protein, 11 aa, synthetic construct
AQLNTTKSVSQ

SEQ ID NO: 195 - protein, 11 aa, synthetic construct
AQLNTTKIVAQ

SEQ ID NO: 196 - protein, 11 aa, synthetic construct
PQLNTTKSVMS

SEQ ID NO: 197 - protein, 11 aa, synthetic construct
PQLNTTKSVQQ

SEQ ID NO: 198 - protein, 11 aa, synthetic construct
AQLNTTKNQMQ

SEQ ID NO: 199 - protein, 11 aa, synthetic construct
AQLNTTKAVMQ

SEQ ID NO: 200 - protein, 11 aa, synthetic construct
AQNNTTKAVMQ

SEQ ID NO: 201 - protein, 11 aa, synthetic construct
AILNTTKSVNQ

SEQ ID NO: 202 - protein, 11 aa, synthetic construct
AQLNTTKSFNQ

SEQ ID NO: 203 - protein, 11 aa, synthetic construct
AQSNTTKNVMQ

SEQ ID NO: 204 - protein, 11 aa, synthetic construct
AQLNTTKSVSG

SEQ ID NO: 205 - protein, 11 aa, synthetic construct
ESLNTTKSVMG

SEQ ID NO: 206 - protein, 11 aa, synthetic construct
AQANTTKSVMN

SEQ ID NO: 207 - protein, 11 aa, synthetic construct
AQLNTTKSVPN

SEQ ID NO: 208 - protein, 11 aa, synthetic construct
AQDNTTKSIMQ

SEQ ID NO: 209 - protein, 11 aa, synthetic construct
AQLNTTKNVMS

SEQ ID NO: 210 - protein, 11 aa, synthetic construct
AQDNTTKSVGQ

SEQ ID NO: 211 - protein, 11 aa, synthetic construct
QDLNTTKSVMG

SEQ ID NO: 212 - protein, 11 aa, synthetic construct
AQNNTTKSVSQ

SEQ ID NO: 213 - protein, 11 aa, synthetic construct
AQGNITKSVMQ

SEQ ID NO: 214 - protein, 11 aa, synthetic construct
AQLNTTKSVQD

SEQ ID NO: 215 - protein, 11 aa, synthetic construct
ASGNTTKSVMQ

SEQ ID NO: 216 - protein, 11 aa, synthetic construct
AQQNTTKSVMS

SEQ ID NO: 217 - protein, 11 aa, synthetic construct
AQSNTTKSIMQ

SEQ ID NO: 218 - protein, 11 aa, synthetic construct
AQLNTTKIVNQ

SEQ ID NO: 219 - protein, 11 aa, synthetic construct
AQGNTTKIVMQ

SEQ ID NO: 220 - protein, 11 aa, synthetic construct
SQDNTTKSVMQ

SEQ ID NO: 221 - protein, 11 aa, synthetic construct
AQANTTKSVGQ

SEQ ID NO: 222 - protein, 11 aa, synthetic construct
DQLNTTKAVMQ

SEQ ID NO: 223 - protein, 11 aa, synthetic construct
ADINTTKSVMQ

SEQ ID NO: 224 - protein, 11 aa, synthetic construct
PQINTTKSVMQ

SEQ ID NO: 225 - protein, 11 aa, synthetic construct
AQLNTTKAVMN

SEQ ID NO: 226 - protein, 11 aa, synthetic construct
ADLNTTKSVML

SEQ ID NO: 227 - protein, 11 aa, synthetic construct
DQLNTTKSFMQ

SEQ ID NO: 228 - protein, 11 aa, synthetic construct
AQLNTTKPFMQ

SEQ ID NO: 229 - protein, 11 aa, synthetic construct
AQANTTKNVMQ

SEQ ID NO: 230 - protein, 11 aa, synthetic construct
ADLNTTKSVMH

SEQ ID NO: 231 - protein, 11 aa, synthetic construct
AILNTTKSVQQ

SEQ ID NO: 232 - protein, 11 aa, synthetic construct
IQLNTTKSVNQ

SEQ ID NO: 233 - protein, 11 aa, synthetic construct
AQSNTTKIVMQ

SEQ ID NO: 234 - protein, 11 aa, synthetic construct
AQGNTTKSVMI

SEQ ID NO: 235 - protein, 11 aa, synthetic construct
EPLNTTKSVMQ

SEQ ID NO: 236 - protein, 11 aa, synthetic construct
AQQNTTKSVGQ

SEQ ID NO: 237 - protein, 11 aa, synthetic construct
DQNNTTKSVMQ

SEQ ID NO: 238 - protein, 11 aa, synthetic construct
AQLNTTKPVPQ

SEQ ID NO: 239 - protein, 11 aa, synthetic construct
AQLNTTKSVMN

SEQ ID NO: 240 - protein, 11 aa, synthetic construct
AQLNTTKIVFQ

SEQ ID NO: 241 - protein, 11 aa, synthetic construct
AQLNTTKSVSS

SEQ ID NO: 242 - protein, 11 aa, synthetic construct
AQLNTTKPIMQ

SEQ ID NO: 243 - protein, 11 aa, synthetic construct
AQLNTTKNVQQ

SEQ ID NO: 244 - protein, 11 aa, synthetic construct
AQLNTTKSIMN

SEQ ID NO: 245 - protein, 11 aa, synthetic construct
HDLNTTKSVMQ

SEQ ID NO: 246 - protein, 11 aa, synthetic construct
MDLNTTKSVMQ

SEQ ID NO: 247 - protein, 11 aa, synthetic construct
QQLNTTKSVNQ

SEQ ID NO: 248 - protein, 11 aa, synthetic construct
AQLNTTKNIMQ

SEQ ID NO: 249 - protein, 11 aa, synthetic construct
AQLNTTKQVMG

SEQ ID NO: 250 - protein, 11 aa, synthetic construct
AQLNTTKIVMD

SEQ ID NO: 251 - protein, 11 aa, synthetic construct
AQDNTTKSAMQ

SEQ ID NO: 252 - protein, 11 aa, synthetic construct
ADLNTTKGVMQ

SEQ ID NO: 253 - protein, 11 aa, synthetic construct
GQLNTTKSVNQ

SEQ ID NO: 254 - protein, 11 aa, synthetic construct
AQINTTKQVMQ

SEQ ID NO: 255 - protein, 11 aa, synthetic construct
MELNTTKSVMQ

SEQ ID NO: 256 - protein, 11 aa, synthetic construct
DALNTTKSVMG

SEQ ID NO: 257 - protein, 11 aa, synthetic construct
DQLNTTKNVMQ

SEQ ID NO: 258 - protein, 11 aa, synthetic construct
AALNTTKSVNQ

SEQ ID NO: 259 - protein, 11 aa, synthetic construct
PALNTTKSVMQ

SEQ ID NO: 260 - protein, 11 aa, synthetic construct
ANLNTTKAVMQ

SEQ ID NO: 261 - protein, 11 aa, synthetic construct
AQLNTTKNVNQ

SEQ ID NO: 262 - protein, 11 aa, synthetic construct
APGNTTKSVMQ

SEQ ID NO: 263 - protein, 11 aa, synthetic construct
AQNNTTKSVMN

SEQ ID NO: 264 - protein, 11 aa, synthetic construct
AQQNTTKPVMQ

SEQ ID NO: 265 - protein, 11 aa, synthetic construct
AQLNTTKQVMD

SEQ ID NO: 266 - protein, 11 aa, synthetic construct
AALNTTKSIMQ

SEQ ID NO: 267 - protein, 11 aa, synthetic construct
AQLNTTKSIMG

SEQ ID NO: 268 - protein, 11 aa, synthetic construct
GQDNTTKSVMQ

SEQ ID NO: 269 - protein, 11 aa, synthetic construct
ANLNTTKSVNQ

SEQ ID NO: 270 - protein, 11 aa, synthetic construct
AMLNTTKSVMQ

SEQ ID NO: 271 - protein, 11 aa, synthetic construct
GQLNTTKQVMQ

SEQ ID NO: 272 - protein, 11 aa, synthetic construct
QQGNTTKSVMQ

SEQ ID NO: 273 - protein, 11 aa, synthetic construct
AQLNTTKSFGQ

SEQ ID NO: 274 - protein, 11 aa, synthetic construct
AQLNTTKQVMN

SEQ ID NO: 275 - protein, 11 aa, synthetic construct
AQLNTTKIVMG

SEQ ID NO: 276 - protein, 11 aa, synthetic construct
DALNTTKSVMS

SEQ ID NO: 277 - protein, 11 aa, synthetic construct
APLNTTKSVNQ

SEQ ID NO: 278 - protein, 11 aa, synthetic construct
AQLNTTKSIGQ

SEQ ID NO: 279 - protein, 11 aa, synthetic construct
AQLNTTKSVGN

SEQ ID NO: 280 - protein, 11 aa, synthetic construct
ASLNTTKSVMG

SEQ ID NO: 281 - protein, 11 aa, synthetic construct
AQSNTTKSVMN

SEQ ID NO: 282 - protein, 11 aa, synthetic construct
AQANTTKSIMQ

SEQ ID NO: 283 - protein, 11 aa, synthetic construct
AQANTTKSVMA

SEQ ID NO: 284 - protein, 11 aa, synthetic construct
AALNTTKNVMQ

SEQ ID NO: 285 - protein, 11 aa, synthetic construct
AQNNTTKSVAQ

SEQ ID NO: 286 - protein, 11 aa, synthetic construct
AQLNTTKSSNQ

SEQ ID NO: 287 - protein, 11 aa, synthetic construct
SQLNTTKNVMQ

SEQ ID NO: 288 - protein, 11 aa, synthetic construct
AQLNTTKQVFQ

SEQ ID NO: 289 - protein, 11 aa, synthetic construct
AQLNTTKAVMD

SEQ ID NO: 290 - protein, 11 aa, synthetic construct
ADLNTTKSFMQ

SEQ ID NO: 291 - protein, 11 aa, synthetic construct
AQNNTTKSVMQ

SEQ ID NO: 292 - protein, 11 aa, synthetic construct
AQLNTTKAIMQ

SEQ ID NO: 293 - protein, 11 aa, synthetic construct
AQANTTKSVMS

SEQ ID NO: 294 - protein, 11 aa, synthetic construct
AQQNTTKSVPQ

SEQ ID NO: 295 - protein, 11 aa, synthetic construct
QQLNTTKSVMG

SEQ ID NO: 296 - protein, 11 aa, synthetic construct
AQLNTTKIVMN

SEQ ID NO: 297 - protein, 11 aa, synthetic construct
AQLNTTKSVQA

SEQ ID NO: 298 - protein, 11 aa, synthetic construct
AQLNTTKSVSA

SEQ ID NO: 299 - protein, 11 aa, synthetic construct
AQLNTTKPVMD

SEQ ID NO: 300 - protein, 11 aa, synthetic construct
AQLNTTKPVNQ

SEQ ID NO: 301 - protein, 11 aa, synthetic construct
DQLNTTKSHMQ

SEQ ID NO: 302 - protein, 11 aa, synthetic construct
AQLNTTKIVQQ

SEQ ID NO: 303 - protein, 11 aa, synthetic construct
EFLNTTKSVMS

SEQ ID NO: 304 - protein, 11 aa, synthetic construct
IQLNTTKSVGQ

SEQ ID NO: 305 - protein, 11 aa, synthetic construct
PMLNTTKSVME

SEQ ID NO: 306 - protein, 11 aa, synthetic construct
AQLNTTKSVGP

SEQ ID NO: 307 - protein, 11 aa, synthetic construct
PQLNTTKSVNQ

SEQ ID NO: 308 - protein, 11 aa, synthetic construct
IQQNTTKSVMQ

SEQ ID NO: 309 - protein, 11 aa, synthetic construct
ANLNTTKSVDQ

SEQ ID NO: 310 - protein, 11 aa, synthetic construct
PQLNTTKSVMP

SEQ ID NO: 311 - protein, 11 aa, synthetic construct
DSLNTTKSVMN

SEQ ID NO: 312 - protein, 11 aa, synthetic construct
AQLNTTKSVFQ

SEQ ID NO: 313 - protein, 11 aa, synthetic construct
MDLNTTKSVMH

SEQ ID NO: 314 - protein, 11 aa, synthetic construct
HDLNTTKSVMI

SEQ ID NO: 315 - protein, 11 aa, synthetic construct
AQQNTTKSVSQ

SEQ ID NO: 316 - protein, 11 aa, synthetic construct
DQLNTTKQVMQ

SEQ ID NO: 317 - protein, 11 aa, synthetic construct
AQLNTTKSVGD

SEQ ID NO: 318 - protein, 11 aa, synthetic construct
AQLNTTKPVDQ

SEQ ID NO: 319 - protein, 11 aa, synthetic construct
ASLNTTKQVMQ

SEQ ID NO: 320 - protein, 11 aa, synthetic construct
ALLNTTKSVMG

SEQ ID NO: 321 - protein, 11 aa, synthetic construct
AQGNTTKSVMN

SEQ ID NO: 322 - protein, 11 aa, synthetic construct
ALLNTTKSVMN

SEQ ID NO: 323 - protein, 11 aa, synthetic construct
AQLNTTKSVFP

SEQ ID NO: 324 - protein, 11 aa, synthetic construct
IQLNTTKSVSQ

SEQ ID NO: 325 - protein, 11 aa, synthetic construct
AQLNTTKAVMG

SEQ ID NO: 326 - protein, 11 aa, synthetic construct
AILNTTKSVMG

SEQ ID NO: 327 - protein, 11 aa, synthetic construct
AQLNTTKDVSQ

SEQ ID NO: 328 - protein, 11 aa, synthetic construct
PALNTTKSVMS

SEQ ID NO: 329 - protein, 11 aa, synthetic construct
PQHNTTKSVMQ

SEQ ID NO: 330 - protein, 11 aa, synthetic construct
AQQNTTKSVMI

SEQ ID NO: 331 - protein, 11 aa, synthetic construct
AQLNTTKSVMS

SEQ ID NO: 332 - protein, 11 aa, synthetic construct
PQLNTTKSVMI

SEQ ID NO: 333 - protein, 11 aa, synthetic construct
AILNTTKQVMQ

SEQ ID NO: 334 - protein, 11 aa, synthetic construct
AQLNTTKSFQQ

SEQ ID NO: 335 - protein, 11 aa, synthetic construct
QQLNTTKSVSQ

SEQ ID NO: 336 - protein, 11 aa, synthetic construct
AQLNTTKPVSQ

SEQ ID NO: 337 - protein, 11 aa, synthetic construct
QELNTTKSVMI

SEQ ID NO: 338 - protein, 11 aa, synthetic construct
ANLNTTKQVMQ

SEQ ID NO: 339 - protein, 11 aa, synthetic construct
QQLNTTKPVMQ

SEQ ID NO: 340 - protein, 11 aa, synthetic construct
AQINTTKPVMQ

SEQ ID NO: 341 - protein, 11 aa, synthetic construct
HQLNTTKSVDQ

SEQ ID NO: 342 - protein, 11 aa, synthetic construct
QQLNTTKQVMQ

SEQ ID NO: 343 - protein, 11 aa, synthetic construct
IQLNTTKSVQQ

SEQ ID NO: 344 - protein, 11 aa, synthetic construct
AQLNTTKSVGS

SEQ ID NO: 345 - protein, 11 aa, synthetic construct
AQQNTTKSVMN

SEQ ID NO: 346 - protein, 11 aa, synthetic construct
AQFNTTKSVDQ

SEQ ID NO: 347 - protein, 11 aa, synthetic construct
AQLNTTKNVMG

SEQ ID NO: 348 - protein, 11 aa, synthetic construct
AQLNTTKNVMN

SEQ ID NO: 349 - protein, 11 aa, synthetic construct
AQHNTTKAVMQ

SEQ ID NO: 350 - protein, 11 aa, synthetic construct
AQLNTTKSVSD

SEQ ID NO: 351 - protein, 11 aa, synthetic construct
PLLNTTKSVME

SEQ ID NO: 352 - protein, 11 aa, synthetic construct
AQNNTTKSIMQ

SEQ ID NO: 353 - protein, 11 aa, synthetic construct
PSLNTTKSVMQ

SEQ ID NO: 354 - protein, 11 aa, synthetic construct
AQLNTTKDVNQ

SEQ ID NO: 355 - protein, 11 aa, synthetic construct
ASLNTTKAVMQ

SEQ ID NO: 356 - protein, 11 aa, synthetic construct
AQLNTTKSVSN

SEQ ID NO: 357 - protein, 11 aa, synthetic construct
AELNTTKSVMG

SEQ ID NO: 358 - protein, 11 aa, synthetic construct
AQDNTTKSVMG

SEQ ID NO: 359 - protein, 11 aa, synthetic construct
AGLNTTKNVMQ

SEQ ID NO: 360 - protein, 11 aa, synthetic construct
AQLNTTKSVGI

SEQ ID NO: 361 - protein, 11 aa, synthetic construct
PQLNTTKSNMQ

SEQ ID NO: 362 - protein, 11 aa, synthetic construct
AQPNTTKPVMQ

SEQ ID NO: 363 - protein, 11 aa, synthetic construct
AQLNTTKSIAQ

SEQ ID NO: 364 - protein, 11 aa, synthetic construct
AQLNTTKPHMQ

SEQ ID NO: 365 - protein, 11 aa, synthetic construct
AQLNTTKSVMM

SEQ ID NO: 366 - protein, 11 aa, synthetic construct
AQANTTKSVMD

SEQ ID NO: 367 - protein, 11 aa, synthetic construct
AQLNTTKQVMS

SEQ ID NO: 368 - protein, 11 aa, synthetic construct
PQLNTTKSVMD

SEQ ID NO: 369 - protein, 11 aa, synthetic construct
QELNTTKSVMS

SEQ ID NO: 370 - protein, 11 aa, synthetic construct
AQINTTKSVNQ

SEQ ID NO: 371 - protein, 11 aa, synthetic construct
AQLNTTKPVMN

SEQ ID NO: 372 - protein, 11 aa, synthetic construct
AQLNTTKQFMQ

SEQ ID NO: 373 - protein, 11 aa, synthetic construct
AMLNTTKSVMA

SEQ ID NO: 374 - protein, 11 aa, synthetic construct
AQLNTTKNAMQ

SEQ ID NO: 375 - protein, 11 aa, synthetic construct
PDLNTTKSVMG

SEQ ID NO: 376 - protein, 11 aa, synthetic construct
PMLNTTKSVMM

SEQ ID NO: 377 - protein, 11 aa, synthetic construct
AALNTTKSVMG

SEQ ID NO: 378 - protein, 11 aa, synthetic construct
EMLNTTKSVMQ

SEQ ID NO: 379 - protein, 11 aa, synthetic construct
NQANTTKSVMQ

SEQ ID NO: 380 - protein, 11 aa, synthetic construct
AILNTTKNVMQ

SEQ ID NO: 381 - protein, 11 aa, synthetic construct
ASLNTTKSVQQ

SEQ ID NO: 382 - protein, 11 aa, synthetic construct
DQLNTTKIVMQ

SEQ ID NO: 383 - protein, 11 aa, synthetic construct
AQLNTTKSFMS

SEQ ID NO: 384 - protein, 11 aa, synthetic construct
DQLNTTKSQMQ

SEQ ID NO: 385 - protein, 11 aa, synthetic construct
IDLNTTKSVMQ

SEQ ID NO: 386 - protein, 11 aa, synthetic construct
AQLNTTKAVAQ

SEQ ID NO: 387 - protein, 11 aa, synthetic construct
ANLNTTKPVMQ

SEQ ID NO: 388 - protein, 11 aa, synthetic construct
AQLNTTKSVFA

SEQ ID NO: 389 - protein, 11 aa, synthetic construct
MSLNTTKSVMP

SEQ ID NO: 390 - protein, 11 aa, synthetic construct
QQLNTTKAVMQ

SEQ ID NO: 391 - protein, 11 aa, synthetic construct
PLLNTTKSVMM

SEQ ID NO: 392 - protein, 11 aa, synthetic construct
PQDNTTKSVMQ

SEQ ID NO: 393 - protein, 11 aa, synthetic construct
NQSNTTKSVMQ

SEQ ID NO: 394 - protein, 11 aa, synthetic construct
DQLNTTKSIMQ

SEQ ID NO: 395 - protein, 11 aa, synthetic construct
ASLNTTKSVMA

SEQ ID NO: 396 - protein, 11 aa, synthetic construct
EQLNTTKSVMI

SEQ ID NO: 397 - protein, 11 aa, synthetic construct
GQLNTTKSVMG

SEQ ID NO: 398 - protein, 11 aa, synthetic construct
ADSNTTKSVMQ

SEQ ID NO: 399 - protein, 7 aa, synthetic construct
NGSGQNQ

SEQ ID NO: 400 - protein, 7 aa, synthetic construct
NGEATQG

SEQ ID NO: 401 - protein, 7 aa, synthetic construct
SVENQAK

SEQ ID NO: 402 - protein, 7 aa, synthetic construct
TDDTHKT

SEQ ID NO: 403 - protein, 7 aa, synthetic construct
NTDSTIA

SEQ ID NO: 404 - protein, 7 aa, synthetic construct
DENNHTG

SEQ ID NO: 405 - protein, 7 aa, synthetic construct
TNNGDNK

SEQ ID NO: 406 - protein, 7 aa, synthetic construct
GAGNENA

SEQ ID NO: 407 - protein, 7 aa, synthetic construct
HPDDNNK

SEQ ID NO: 408 - protein, 7 aa, synthetic construct
DDNGGLK

SEQ ID NO: 409 - protein, 7 aa, synthetic construct
SPGNDNQ

SEQ ID NO: 410 - protein, 7 aa, synthetic construct
QEGNEMV

SEQ ID NO: 411 - protein, 7 aa, synthetic construct
NNDNSVS

SEQ ID NO: 412 - protein, 7 aa, synthetic construct
QPDQSPS

SEQ ID NO: 413 - protein, 7 aa, synthetic construct
DNTDGHP

SEQ ID NO: 414 - protein, 7 aa, synthetic construct
ADQDSSS

SEQ ID NO: 415 - protein, 7 aa, synthetic construct
QEDNQQS

SEQ ID NO: 416 - protein, 7 aa, synthetic construct
EQQAGAK

SEQ ID NO: 417 - protein, 7 aa, synthetic construct
QQNEHGV

SEQ ID NO: 418 - protein, 7 aa, synthetic construct
ELGNKQA

SEQ ID NO: 419 - protein, 7 aa, synthetic construct
VGEHQNM

SEQ ID NO: 420 - protein, 7 aa, synthetic construct
QQENKDQ

SEQ ID NO: 421 - protein, 7 aa, synthetic construct
DNNKTDH

SEQ ID NO: 422 - protein, 7 aa, synthetic construct
DTDTKHT

SEQ ID NO: 423 - protein, 7 aa, synthetic construct
GEGRSNA

SEQ ID NO: 424 - protein, 7 aa, synthetic construct
SMENEAR

SEQ ID NO: 425 - protein, 7 aa, synthetic construct
DAADGSK

SEQ ID NO: 426 - protein, 7 aa, synthetic construct
EEESHPK

SEQ ID NO: 427 - protein, 7 aa, synthetic construct
VQDNQHR

SEQ ID NO: 428 - protein, 7 aa, synthetic construct
DRDSATN

SEQ ID NO: 429 - protein, 7 aa, synthetic construct
NGTHDQK

SEQ ID NO: 430 - protein, 7 aa, synthetic construct
NTTGKND

SEQ ID NO: 431 - protein, 7 aa, synthetic construct
KDGTPNN

SEQ ID NO: 432 - protein, 7 aa, synthetic construct
NGVKGNE

SEQ ID NO: 433 - protein, 7 aa, synthetic construct
GETRGTE

SEQ ID NO: 434 - protein, 7 aa, synthetic construct
QEQVKSV

SEQ ID NO: 435 - protein, 7 aa, synthetic construct
TDDQGSQ

SEQ ID NO: 436 - protein, 7 aa, synthetic construct
NGDGKGD

SEQ ID NO: 437 - protein, 7 aa, synthetic construct
HTGNKTD

SEQ ID NO: 438 - protein, 7 aa, synthetic construct
SPGTSQE

SEQ ID NO: 439 - protein, 7 aa, synthetic construct
NVNDNNR

SEQ ID NO: 440 - protein, 7 aa, synthetic construct
DTASGQM

SEQ ID NO: 441 - protein, 7 aa, synthetic construct
DSLGKQE

SEQ ID NO: 442 - protein, 7 aa, synthetic construct
GGQSPAQ

SEQ ID NO: 443 - protein, 7 aa, synthetic construct
DLQEGSK

SEQ ID NO: 444 - protein, 7 aa, synthetic construct
QEGKNNQ

SEQ ID NO: 445 - protein, 7 aa, synthetic construct
GGGSQQQ

SEQ ID NO: 446 - protein, 7 aa, synthetic construct
DSTVSRN

SEQ ID NO: 447 - protein, 7 aa, synthetic construct
NGVNNPT

SEQ ID NO: 448 - protein, 7 aa, synthetic construct
DGAPRSE

SEQ ID NO: 449 - protein, 7 aa, synthetic construct
SVDEHGK

SEQ ID NO: 450 - protein, 7 aa, synthetic construct
TQDASPV

SEQ ID NO: 451 - protein, 7 aa, synthetic construct
TNETKTM

SEQ ID NO: 452 - protein, 7 aa, synthetic construct
ASDSHTV

SEQ ID NO: 453 - protein, 7 aa, synthetic construct
DANQKGG

SEQ ID NO: 454 - protein, 7 aa, synthetic construct
SNAQNMQ

SEQ ID NO: 455 - protein, 7 aa, synthetic construct
EGQHRQE

SEQ ID NO: 456 - protein, 7 aa, synthetic construct
VATTEQA

SEQ ID NO: 457 - protein, 7 aa, synthetic construct
NTGPNPT

SEQ ID NO: 458 - protein, 7 aa, synthetic construct
HGSGSEG

SEQ ID NO: 459 - protein, 7 aa, synthetic construct
DHGNHPT

SEQ ID NO: 460 - protein, 7 aa, synthetic construct
SDTVQHP

SEQ ID NO: 461 - protein, 7 aa, synthetic construct
NSTPGNT

SEQ ID NO: 462 - protein, 7 aa, synthetic construct
EHSVGDQ

SEQ ID NO: 463 - protein, 7 aa, synthetic construct
SQTAQDK

SEQ ID NO: 464 - protein, 7 aa, synthetic construct
ENNPNLK

SEQ ID NO: 465 - protein, 7 aa, synthetic construct
GEDKAHV

SEQ ID NO: 466 - protein, 7 aa, synthetic construct
GQTAETA

SEQ ID NO: 467 - protein, 7 aa, synthetic construct
GELAGMQ

SEQ ID NO: 468 - protein, 7 aa, synthetic construct
SDGQAQR

SEQ ID NO: 469 - protein, 7 aa, synthetic construct
EQKQAVQ

SEQ ID NO: 470 - protein, 7 aa, synthetic construct
QQEPANK

SEQ ID NO: 471 - protein, 7 aa, synthetic construct
NDTPTIG

SEQ ID NO: 472 - protein, 7 aa, synthetic construct
LQGGGET

SEQ ID NO: 473 - protein, 7 aa, synthetic construct
EKDRPNM

SEQ ID NO: 474 - protein, 7 aa, synthetic construct
DEHKSKT

SEQ ID NO: 475 - protein, 7 aa, synthetic construct
DQGTRMS

SEQ ID NO: 476 - protein, 7 aa, synthetic construct
DTPSNNA

SEQ ID NO: 477 - protein, 7 aa, synthetic construct
QTLGQQQ

SEQ ID NO: 478 - protein, 7 aa, synthetic construct
NEEHVSK

SEQ ID NO: 479 - protein, 7 aa, synthetic construct
GDAKTMM

SEQ ID NO: 480 - protein, 7 aa, synthetic construct
QMRTDQE

SEQ ID NO: 481 - protein, 7 aa, synthetic construct
KADTTNQ

SEQ ID NO: 482 - protein, 7 aa, synthetic construct
GGNEDVN

SEQ ID NO: 483 - protein, 7 aa, synthetic construct
NHTDTGG

SEQ ID NO: 484 - protein, 7 aa, synthetic construct
GESTAGH

SEQ ID NO: 485 - protein, 7 aa, synthetic construct
GDLQSTG

SEQ ID NO: 486 - protein, 7 aa, synthetic construct
NADTVEM

SEQ ID NO: 487 - protein, 7 aa, synthetic construct
NEDGHQE

SEQ ID NO: 488 - protein, 7 aa, synthetic construct
TDERTVN

SEQ ID NO: 489 - protein, 7 aa, synthetic construct
NVEMPTL

SEQ ID NO: 490 - protein, 7 aa, synthetic construct
AMSQAQT

SEQ ID NO: 491 - protein, 7 aa, synthetic construct
ENAEGQR

SEQ ID NO: 492 - protein, 7 aa, synthetic construct
TEKNQQV

SEQ ID NO: 493 - protein, 7 aa, synthetic construct
SPDHKTV

SEQ ID NO: 494 - protein, 7 aa, synthetic construct
MQQASAH

SEQ ID NO: 495 - protein, 7 aa, synthetic construct
TGTPGHT

SEQ ID NO: 496 - protein, 7 aa, synthetic construct
DNIGKQT

SEQ ID NO: 497 - protein, 7 aa, synthetic construct
ESESQSN

SEQ ID NO: 498 - protein, 7 aa, synthetic construct
GAQETPG

SEQ ID NO: 499 - protein, 7 aa, synthetic construct
DAASLQT

SEQ ID NO: 500 - protein, 7 aa, synthetic construct
GDAQNQE

SEQ ID NO: 501 - protein, 7 aa, synthetic construct
QGQEHAT

SEQ ID NO: 502 - protein, 7 aa, synthetic construct
DMSQRVM

SEQ ID NO: 503 - protein, 7 aa, synthetic construct
TQEGKGD

SEQ ID NO: 504 - protein, 7 aa, synthetic construct
MNENEGR

SEQ ID NO: 505 - protein, 7 aa, synthetic construct
HQNGQHS

SEQ ID NO: 506 - protein, 7 aa, synthetic construct
ETQNHAR

SEQ ID NO: 507 - protein, 7 aa, synthetic construct
QKDTKEV

SEQ ID NO: 508 - protein, 7 aa, synthetic construct
GDNPPTI

SEQ ID NO: 509 - protein, 7 aa, synthetic construct
AASAGNH

SEQ ID NO: 510 - protein, 7 aa, synthetic construct
ESHAGAQ

SEQ ID NO: 511 - protein, 7 aa, synthetic construct
TLGTGSN

SEQ ID NO: 512 - protein, 7 aa, synthetic construct
DDQHSER

SEQ ID NO: 513 - protein, 7 aa, synthetic construct
DAEQKGR

SEQ ID NO: 514 - protein, 7 aa, synthetic construct
TPASPHV

SEQ ID NO: 515 - protein, 7 aa, synthetic construct
ASQEHSN

SEQ ID NO: 516 - protein, 7 aa, synthetic construct
DAAGAEK

SEQ ID NO: 517 - protein, 7 aa, synthetic construct
LEQTAVH

SEQ ID NO: 518 - protein, 7 aa, synthetic construct
NGPHDGN

SEQ ID NO: 519 - protein, 7 aa, synthetic construct
KTDNHMT

SEQ ID NO: 520 - protein, 7 aa, synthetic construct
THNGDHN

SEQ ID NO: 521 - protein, 7 aa, synthetic construct
VPETTVK

SEQ ID NO: 522 - protein, 7 aa, synthetic construct
GNNTTPI

SEQ ID NO: 523 - protein, 7 aa, synthetic construct
IDDSQMS

SEQ ID NO: 524 - protein, 7 aa, synthetic construct
NTTPHTD

SEQ ID NO: 525 - protein, 7 aa, synthetic construct
AETQIQN

SEQ ID NO: 526 - protein, 7 aa, synthetic construct
QGSTPGI

SEQ ID NO: 527 - protein, 7 aa, synthetic construct
GTDKPGG

SEQ ID NO: 528 - protein, 7 aa, synthetic construct
HSEGEQQ

SEQ ID NO: 529 - protein, 7 aa, synthetic construct
GTESGRD

SEQ ID NO: 530 - protein, 7 aa, synthetic construct
SGQNEHS

SEQ ID NO: 531 - protein, 7 aa, synthetic construct
EQHNRTM

SEQ ID NO: 532 - protein, 7 aa, synthetic construct
LEQGNTN

SEQ ID NO: 533 - protein, 7 aa, synthetic construct
ILENNQQ

SEQ ID NO: 534 - protein, 7 aa, synthetic construct
HDDHGGT

SEQ ID NO: 535 - protein, 7 aa, synthetic construct
NVEKADT

SEQ ID NO: 536 - protein, 7 aa, synthetic construct
SGMSGTQ

SEQ ID NO: 537 - protein, 7 aa, synthetic construct
HQNVGSE

SEQ ID NO: 538 - protein, 7 aa, synthetic construct
SGERALS

SEQ ID NO: 539 - protein, 7 aa, synthetic construct
SGEARIQ

SEQ ID NO: 540 - protein, 7 aa, synthetic construct
LPGESTP

SEQ ID NO: 541 - protein, 7 aa, synthetic construct
NGGGNYP

SEQ ID NO: 542 - protein, 7 aa, synthetic construct
ESSNGIM

SEQ ID NO: 543 - protein, 7 aa, synthetic construct
STSSGNH

SEQ ID NO: 544 - protein, 7 aa, synthetic construct
GSTNAHQ

SEQ ID NO: 545 - protein, 7 aa, synthetic construct
DNMKQPI

SEQ ID NO: 546 - protein, 7 aa, synthetic construct
ASTRDNA

SEQ ID NO: 547 - protein, 7 aa, synthetic construct
AGEHSRT

SEQ ID NO: 548 - protein, 7 aa, synthetic construct
IGNQGSH

SEQ ID NO: 549 - protein, 7 aa, synthetic construct
APEQIAN

SEQ ID NO: 550 - protein, 7 aa, synthetic construct
KDNEHAN

SEQ ID NO: 551 - protein, 7 aa, synthetic construct
MQGETSP

SEQ ID NO: 552 - protein, 7 aa, synthetic construct
NGPSSIS

SEQ ID NO: 553 - protein, 7 aa, synthetic construct
GGGPTQS

SEQ ID NO: 554 - protein, 7 aa, synthetic construct
GVAGQDA

SEQ ID NO: 555 - protein, 7 aa, synthetic construct
TQSSERA

SEQ ID NO: 556 - protein, 7 aa, synthetic construct
EQHQPNT

SEQ ID NO: 557 - protein, 7 aa, synthetic construct
DTHPGNR

SEQ ID NO: 558 - protein, 7 aa, synthetic construct
GSDRNVH

SEQ ID NO: 559 - protein, 7 aa, synthetic construct
STVDKTA

SEQ ID NO: 560 - protein, 7 aa, synthetic construct
QGPKSTD

SEQ ID NO: 561 - protein, 7 aa, synthetic construct
DTSTKIQ

SEQ ID NO: 562 - protein, 7 aa, synthetic construct
SSGQSIT

SEQ ID NO: 563 - protein, 7 aa, synthetic construct
QKIQSDV

SEQ ID NO: 564 - protein, 7 aa, synthetic construct
AQTEPIG

SEQ ID NO: 565 - protein, 7 aa, synthetic construct
DTMRGQA

SEQ ID NO: 566 - protein, 7 aa, synthetic construct
AEGKMNN

SEQ ID NO: 567 - protein, 7 aa, synthetic construct
VHNQAAQ

SEQ ID NO: 568 - protein, 7 aa, synthetic construct
GDDVRVV

SEQ ID NO: 569 - protein, 7 aa, synthetic construct
GEQSHAH

SEQ ID NO: 570 - protein, 7 aa, synthetic construct
NSQATLG

SEQ ID NO: 571 - protein, 7 aa, synthetic construct
QTNHKDE

SEQ ID NO: 572 - protein, 7 aa, synthetic construct
AEDRGLE

SEQ ID NO: 573 - protein, 7 aa, synthetic construct
VTNQDIK

SEQ ID NO: 574 - protein, 7 aa, synthetic construct
ANNTPSI

SEQ ID NO: 575 - protein, 7 aa, synthetic construct
YTESRPE

SEQ ID NO: 576 - protein, 7 aa, synthetic construct
AGDTAQL

SEQ ID NO: 577 - protein, 7 aa, synthetic construct
SDSPTQG

SEQ ID NO: 578 - protein, 7 aa, synthetic construct
ETNGAFR

SEQ ID NO: 579 - protein, 7 aa, synthetic construct
HINNRME

SEQ ID NO: 580 - protein, 7 aa, synthetic construct
AEGAYQT

SEQ ID NO: 581 - protein, 7 aa, synthetic construct
GSGNPAN

SEQ ID NO: 582 - protein, 7 aa, synthetic construct
NPKTDHP

SEQ ID NO: 583 - protein, 7 aa, synthetic construct
DEIRGHH

SEQ ID NO: 584 - protein, 7 aa, synthetic construct
QMEKNMS

SEQ ID NO: 585 - protein, 7 aa, synthetic construct
HSDHATR

SEQ ID NO: 586 - protein, 7 aa, synthetic construct
NQLGTQS

SEQ ID NO: 587 - protein, 7 aa, synthetic construct
GTESQNI

SEQ ID NO: 588 - protein, 7 aa, synthetic construct
QNESTNL

SEQ ID NO: 589 - protein, 7 aa, synthetic construct
DNEVGGA

SEQ ID NO: 590 - protein, 7 aa, synthetic construct
MGQKNEA

SEQ ID NO: 591 - protein, 7 aa, synthetic construct
DHGTTVP

SEQ ID NO: 592 - protein, 7 aa, synthetic construct
NTTDPNH

SEQ ID NO: 593 - protein, 7 aa, synthetic construct
KNEHQQL

SEQ ID NO: 594 - protein, 7 aa, synthetic construct
NGAVSMQ

SEQ ID NO: 595 - protein, 7 aa, synthetic construct
TDGKPQN

SEQ ID NO: 596 - protein, 7 aa, synthetic construct
AMSEKNT

SEQ ID NO: 597 - protein, 7 aa, synthetic construct
SSMQRNE

SEQ ID NO: 598 - protein, 7 aa, synthetic construct
GSDQLSH

SEQ ID NO: 599 - protein, 7 aa, synthetic construct
NAPTAAS

SEQ ID NO: 600 - protein, 7 aa, synthetic construct
FTGQKSE

SEQ ID NO: 601 - protein, 7 aa, synthetic construct
NDHVGNS

SEQ ID NO: 602 - protein, 7 aa, synthetic construct
TDSNSTK

SEQ ID NO: 603 - protein, 7 aa, synthetic construct
GNGPNGP

SEQ ID NO: 604 - protein, 7 aa, synthetic construct
ADMQERN

SEQ ID NO: 605 - protein, 7 aa, synthetic construct
FENPPNV

SEQ ID NO: 606 - protein, 7 aa, synthetic construct
EPKDSMT

SEQ ID NO: 607 - protein, 7 aa, synthetic construct
MQNVGQD

SEQ ID NO: 608 - protein, 7 aa, synthetic construct
DVDSLTT

SEQ ID NO: 609 - protein, 7 aa, synthetic construct
NTDNYTK

SEQ ID NO: 610 - protein, 7 aa, synthetic construct
TLGLRQE

SEQ ID NO: 611 - protein, 7 aa, synthetic construct
GVHGENT

SEQ ID NO: 612 - protein, 7 aa, synthetic construct
KLGGNAE

SEQ ID NO: 613 - protein, 7 aa, synthetic construct
AEKVNTM

SEQ ID NO: 614 - protein, 7 aa, synthetic construct
THGTGGD

SEQ ID NO: 615 - protein, 7 aa, synthetic construct
LTEGSGK

SEQ ID NO: 616 - protein, 7 aa, synthetic construct
HYNNQQV

SEQ ID NO: 617 - protein, 7 aa, synthetic construct
HQESSIG

SEQ ID NO: 618 - protein, 7 aa, synthetic construct
KTDGDGP

SEQ ID NO: 619 - protein, 7 aa, synthetic construct
KPDHAHE

SEQ ID NO: 620 - protein, 7 aa, synthetic construct
NIQASTH

SEQ ID NO: 621 - protein, 7 aa, synthetic construct
QVSDNKA

SEQ ID NO: 622 - protein, 7 aa, synthetic construct
GSPPTIV

SEQ ID NO: 623 - protein, 7 aa, synthetic construct
SEAVAMP

SEQ ID NO: 624 - protein, 7 aa, synthetic construct
TIHNGTA

SEQ ID NO: 625 - protein, 7 aa, synthetic construct
SELSENQ

SEQ ID NO: 626 - protein, 7 aa, synthetic construct
NTEAAAD

SEQ ID NO: 627 - protein, 7 aa, synthetic construct
VDGDNYQ

SEQ ID NO: 628 - protein, 7 aa, synthetic construct
GGNTPPG

SEQ ID NO: 629 - protein, 7 aa, synthetic construct
DPDGHHK

SEQ ID NO: 630 - protein, 7 aa, synthetic construct
DNTNPHN

SEQ ID NO: 631 - protein, 7 aa, synthetic construct
SNTTKSV

SEQ ID NO: 632 - protein, 7 aa, synthetic construct
QNTTKSV

SEQ ID NO: 633 - protein, 7 aa, synthetic construct
LNTTKPV

SEQ ID NO: 634 - protein, 7 aa, synthetic construct
ANTTKSV

SEQ ID NO: 635 - protein, 7 aa, synthetic construct
NNTTKSV

SEQ ID NO: 636 - protein, 7 aa, synthetic construct
LNTTKNV

SEQ ID NO: 637 - protein, 7 aa, synthetic construct
LNTTKSV

SEQ ID NO: 638 - protein, 7 aa, synthetic construct
DNTTKSV

SEQ ID NO: 639 - protein, 7 aa, synthetic construct
GNTTKSV

SEQ ID NO: 640 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQSNTTKSVDQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 641 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQQNTTKSVDQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 642 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSADLNTTKPVMQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 643 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQANTTKSVDQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 644 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQNNTTKSVDQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 645 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQLNTTKNVDQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 646 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSADLNTTKSVAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 647 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQDNTTKSVFQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 648 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSADLNTTKSVSQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 649 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQGNTTKSVDQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 650 - DNA, 2211 bp, synthetic construct
  source 1..2211: mol_type = other DNA
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

(19) United States
(12) Patent Application Publication
(10) Pub. No.: US 2025/0346637 A1
(43) Pub. Date: Nov. 13, 2025

(54) ADENO-ASSOCIATED VIRUS COMPOSITIONS HAVING INCREASED BRAIN ENRICHMENT AND DECREASED LIVER ENRICHMENT

(71) Applicant: CAPSIDA, INC., Thousand Oaks, CA (US)

(72) Inventors: Brandon G. Wheeler, Thousand Oaks, CA (US); Troy E. Sandberg, Thousand Oaks, CA (US); Nicholas S. Goeden, Thousand Oaks, CA (US); Nicholas C. Flytzanis, Thousand Oaks, CA (US); Robert W. Sandrock, Thousand Oaks, CA (US)

(21) Appl. No.: 18/867,251

(22) PCT Filed: Jun. 6, 2023
(86) PCT No.: PCT/US2023/067971

§ 371 Date: Nov. 19, 2024

Related U.S. Application Data
(60) Provisional application No. 63/352,845, filed on Jun. 16, 2022.

(51) Int. Cl. C07K 14/005

(52) U.S. Cl. CPC C07K 14/005; C12N 2750/14122; C12N 2750/14143; C12N 2750/14145

(57) ABSTRACT

Described herein are compositions and kits comprising recombinant adeno-associated viruses (rAAVs) with increased transduction enrichment in the brain and, in some cases, reduced transduction in the liver. The rAAV compositions described herein encapsidate a transgene, such as a therapeutic nucleic acid. Gene therapy using the rAAVs is described. Also described are methods of treating brain-related diseases and conditions.

### BACKGROUND

Recombinant adeno-associated viruses (rAAVs) are widely used as vectors for gene delivery in therapeutic applications because of their ability to transduce both dividing and non-dividing cells, their long-term persistence as episomal DNA in infected cells, and their low immunogenicity. These characteristics make them appealing for applications in therapeutic applications, such as gene therapy. However, there is a need to significantly improve the performance of existing AAV serotypes to selectively and efficiently express in distinct cell-types, upon systemic delivery to a subject. This need is especially acute when the AAV must be expressed in the brain.

### SUMMARY OF THE INVENTION

Disclosed herein are rAAVs with peptide insertions and substitutions engineered into the capsid structure through iterative rounds of selection in non-human primates (NHPs), yielding variants having increased transduction when measured in the brain and reduced expression in the liver relative to the wild type rAAV on which the variant is based.

The present invention provides rAAVs with widespread transduction to the brain but reduced transduction to the liver. Following IV injection, unmodified rAAVs such as those derived from AAV9 (SEQ ID NO: 1) may not have sufficient tissue enrichment to treat many human diseases by delivery of an AAV cargo. Directed evolution of AAV9 as described herein has provided modified rAAVs that exhibit increased viral tissue enrichment in the brain. Accordingly, engineered rAAVs described herein are particularly useful in delivering DNA cargo to brain tissue. Moreover, when administering to a patient in quantities high enough to provide effective disease treatment, off-target enrichment in certain tissues like the liver can cause immune response issues. Accordingly, in certain embodiments, modified rAAVs disclosed herein have been selected for not only increased brain transduction but also reduced liver transduction.

The present invention provides, in an aspect, an AAV capsid protein comprising or consisting of an amino-acid sequence set forth in any one of Tables 1-3, FIG. 1 and/or Formula I.

Another aspect of the invention is a modified capsid protein wherein the AAV capsid protein, with a peptide insertion/substitution comprising or consisting of an amino-acid sequence set forth in any one of Tables 1-3, FIG. 1 and/or Formula I, is characterized by increased brain transduction in a subject. In certain aspects a modified capsid protein is provided wherein the AAV capsid protein comprises a peptide insertion/substitution comprising or consisting of an amino-acid sequence set forth in any one of Tables 1-3, FIG. 1 and/or Formula I, is characterized by decreased liver transduction in a subject. Where increased or decreased transduction in a particular tissue is discussed herein, it may be relative to the unmodified or wild type AAV capsid protein

The present disclosure moreover includes pharmaceutical compositions comprising rAAVs with a peptide insertion/substitution comprising or consisting of an amino-acid sequence set forth in any one of Tables 1-3, FIG. 1 and/or Formula I, and a pharmaceutically acceptable excipient.

Aspects disclosed herein provide methods of treating a disease or condition in a subject comprising administering a therapeutically effective amount of a pharmaceutical formulation comprising the AAV capsid protein or the AAV capsid of the present disclosure. In some embodiments, the disease or the condition is a disease or a condition of the brain, and brain of the subject. Relatedly, the invention includes use of the rAAVs in the manufacture of a medicament for treating or preventing the disease or medical condition.

Other aspects of the invention will be apparent from the detailed description and claims that follow.

### BRIEF DESCRIPTION OF THE FIGURES

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows AAV capsid protein insertion and substitution amino acid sequences which were found to have increased brain enrichment and/or decreased liver enrichment in the non-human primate brain relative to the parent capsid.

### DETAILED DESCRIPTION OF THE DISCLOSURE

In certain aspects the disclosure provides modified rAAVs with increased expression levels in the brain along with decreased expression levels in the liver when compared to a parent AAV (e.g., AAV9).

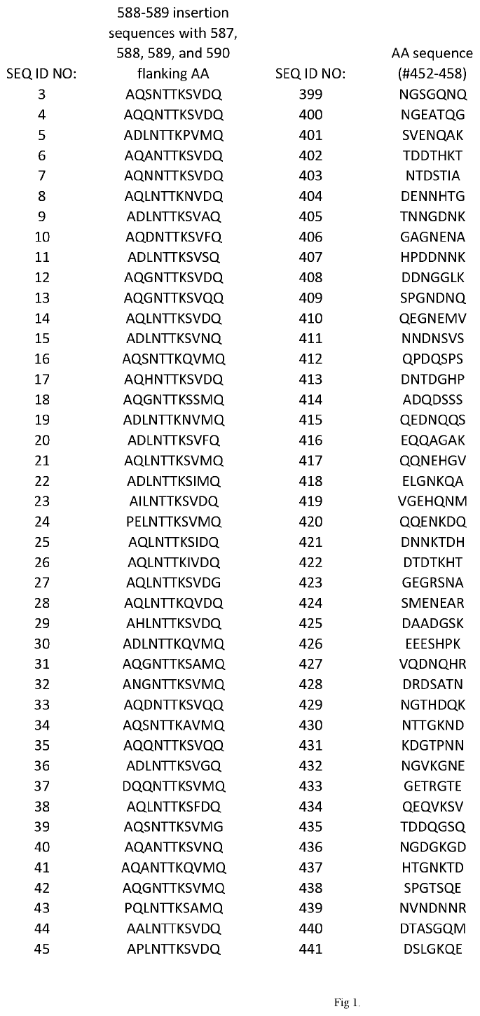

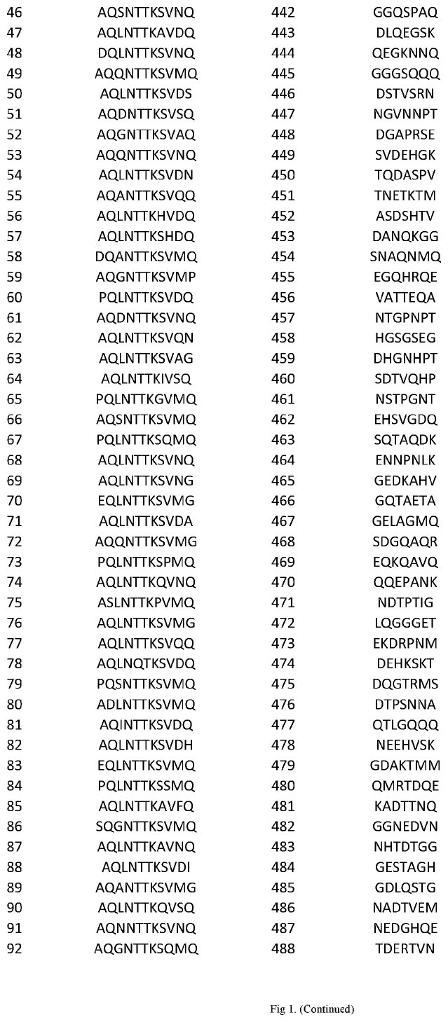

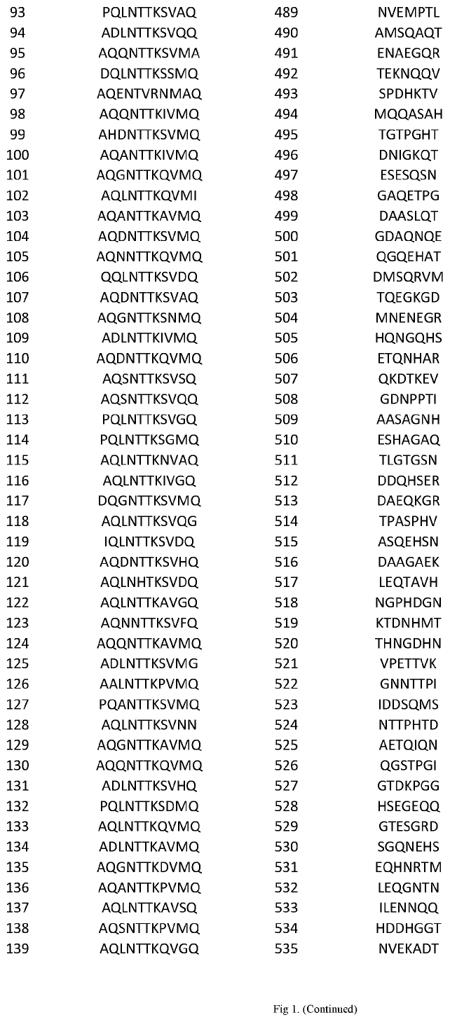

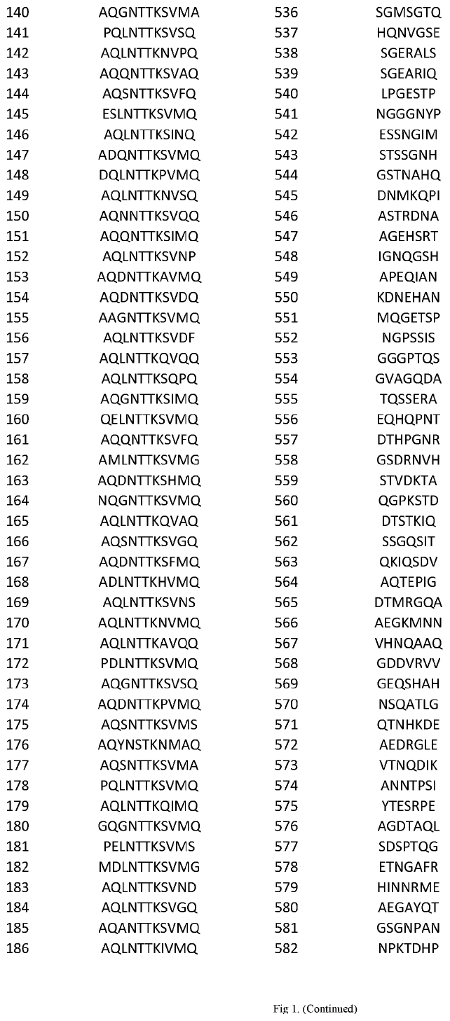

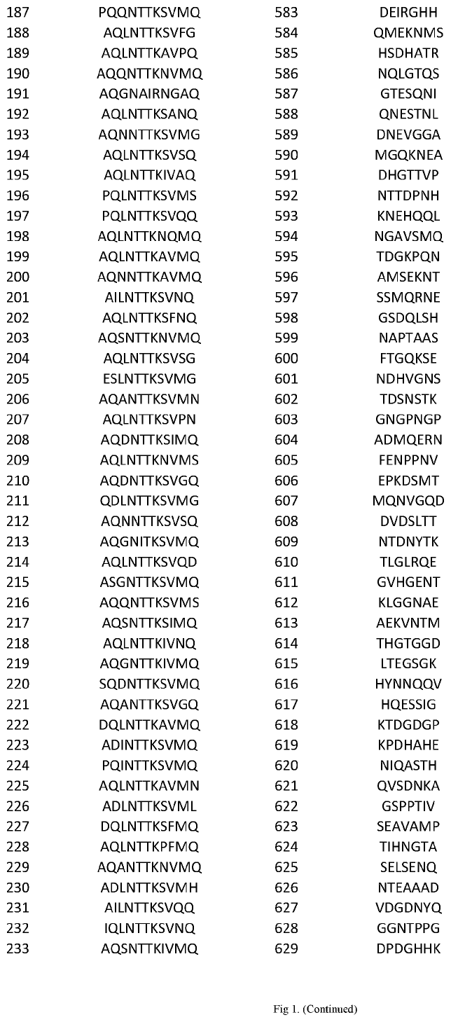

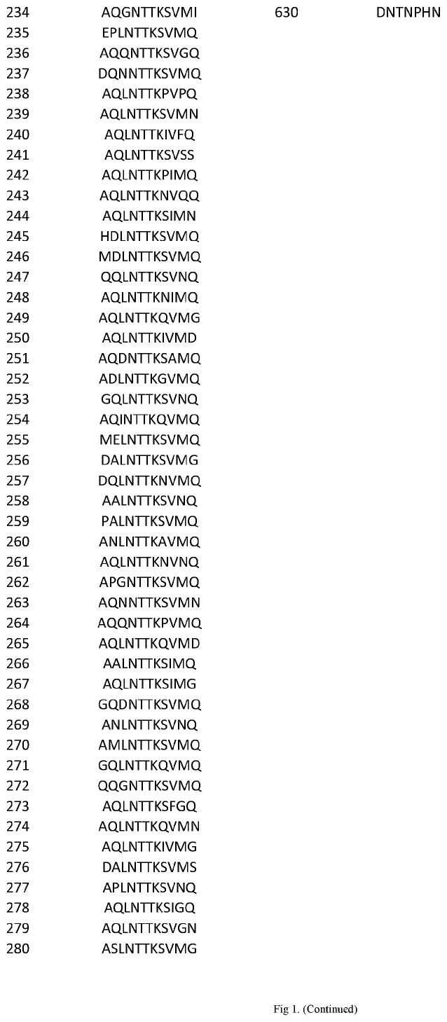

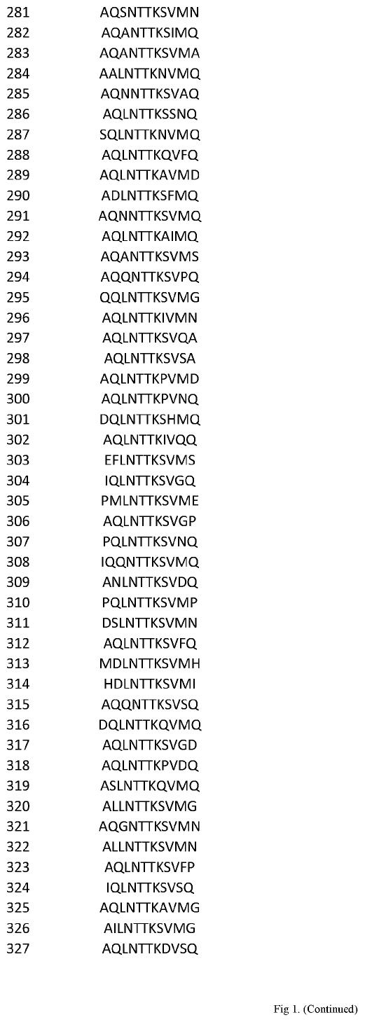

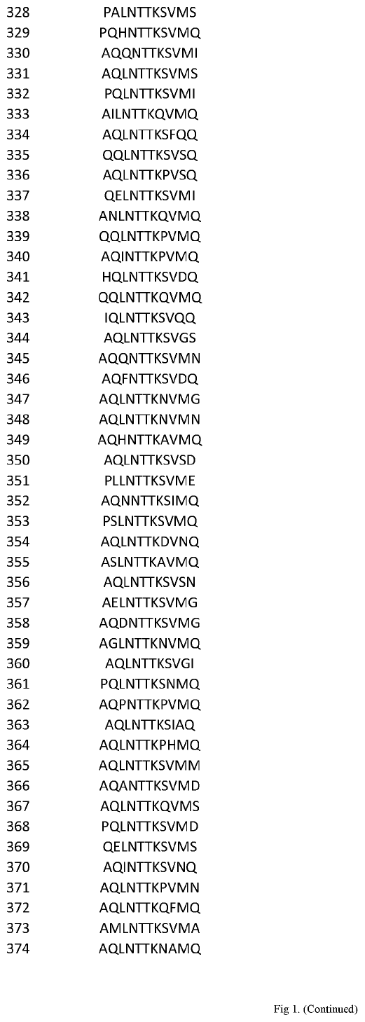

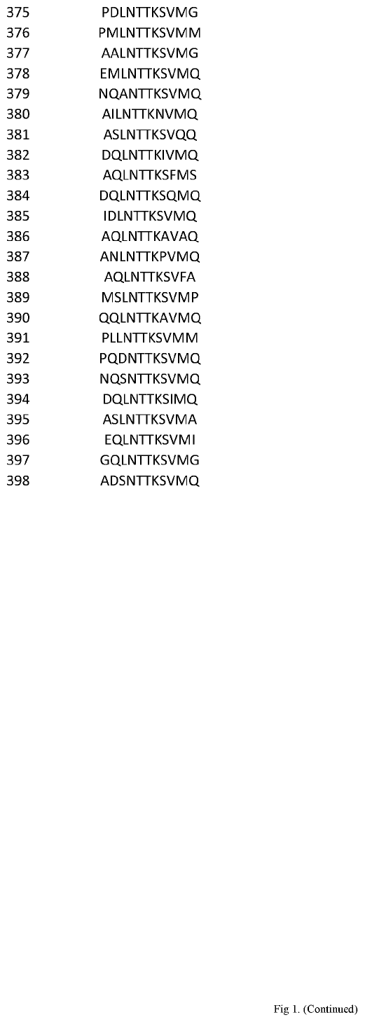

In certain aspects, the disclosure provides rAAVs with a peptide insertion and/or substitution comprising or consisting of an amino-acid sequence set forth in any one of Tables 1-3, FIG. 1 and/or Formula I.

Aspects disclosed herein provide AAV capsids comprising an AAV capsid protein comprising an amino acid sequence of Formula I

(I)
(SEQ ID NO: 2)
X¹-N-T-T-K-X²-X³

wherein X¹ is an amino acid selected from L, G, S, A, Q, and D; X² is an amino acid selected from S, P, Q, and N; and X³ is an amino acid selected from V, A, S, I, and F.

In some embodiments, the AAV capsid protein comprises an amino acid sequence of formula I wherein X¹ is L.

In some embodiments, the AAV capsid protein comprises an amino acid sequence of formula I wherein X¹ is G.

In some embodiments, the AAV capsid protein comprises an amino acid sequence of formula I wherein X² is S.

In some embodiments, the AAV capsid protein comprises an amino acid sequence of formula I wherein X³ is V.

In some embodiments, the peptide insertion and/or substitution sequence is selected from SNTTKSV (SEQ ID NO: 631), QNTTKSV (SEQ ID NO: 632), ADLNTTKPVMQ (SEQ ID NO: 5), AQANTTKSVDQ (SEQ ID NO: 6), AQNNTTKSVDQ (SEQ ID NO: 7), AQLNTTKNVDQ (SEQ ID NO: 8), ADLNTTKSVAQ (SEQ ID NO: 9), AQDNTTKSVFQ (SEQ ID NO: 10), ADLNTTKSVSQ (SEQ ID NO: 11), AQGNTTKSVDQ (SEQ ID NO: 12), AQGNTTKSVQQ (SEQ ID NO: 13), and AQLNTTKSVDQ. (SEQ ID NO: 14).

In some embodiments, the insertion and/or flanking substitution sequences are represented by the peptide sequences listed in Table 1.

TABLE 1

| Sequence | SEQ ID NO | Sequence | SEQ ID NO |
|---|---|---|---|
| SNTTKSV | 631 | AQSNTTKSVDQ | 3 |
| QNTTKSV | 632 | AQQNTTKSVDQ | 4 |
| LNTTKPV | 633 | ADLNTTKPVMQ | 5 |
| ANTTKSV | 634 | AQANTTKSVDQ | 6 |
| NNTTKSV | 635 | AQNNTTKSVDQ | 7 |
| LNTTKNV | 636 | AQLNTTKNVDQ | 8 |
| LNTTKSV | 637 | ADLNTTKSVAQ | 9 |
| DNTTKSV | 638 | AQDNTTKSVFQ | 10 |
| LNTTKSV | 637 | ADLNTTKSVSQ | 11 |
| GNTTKSV | 639 | AQGNTTKSVDQ | 12 |
| GNTTKSV | 639 | AQGNTTKSVQQ | 13 |
| LNTTKSV | 637 | AQLNTTKSVDQ | 14 |

In certain embodiments, the parental AAV is AAV9. In some embodiments, the parental AAV comprises SEQ ID NO: 1. In various embodiments, the AAV capsid protein comprises a 7-mer insertion inserted into the parental AAV between amino acid 588 and amino acid 589 of the parent AAV, wherein positions 587-597 of the AAV capsid protein (with AA position numbering including the insertion) are selected from the sequence provided in Table 1 or selected from the group consisting of SEQ ID NOs: 3-398.

The AAV capsid protein may comprise an amino acid substitution relative to the parental AAV comprising one or more of A587P, Q588D, A589M, A589D, or Q590G.

In some embodiments, the 452-458 sequence is represented by the peptide sequences listed in Table 2 or selected from the group consisting of SEQ ID Nos: 399-630.

TABLE 2

| Sequence | SEQ ID NO |
|---|---|
| NGEATQG | 400 |
| SVENQAK | 401 |
| TDDTHKT | 402 |
| NTDSTIA | 403 |
| DENNHTG | 404 |
| TNNGDNK | 405 |
| GAGNENA | 406 |
| NGSGQNQ | 399 |
| HPDDNNK | 407 |
| DDNGGLK | 408 |
| SPGNDNQ | 409 |
| QEGNEMV | 410 |
| DNTDGHP | 413 |
| ADQDSSS | 414 |
| QEDNQQS | 415 |
| EQQAGAK | 416 |
| QQNEHGV | 417 |
| ELGNKQA | 418 |

Aspects of the invention may include an AAV capsid protein comprising: a sequence provided in Table 1 or selected from the group consisting of SEQ ID Nos: 3-398; and a sequence provided in Table 2 or selected from the group consisting of SEQ ID NOs: 399-630.

Generally, the insertion comprises a five-, six-, or seven-amino acid sequence (5-mer, 6-mer, or 7-mer, respectively) that is inserted or substituted at the 588 loop in a parental AAV capsid protein. Aspects provided herein provide amino acid insertions comprising seven amino acid polymer (7-mer) inserted at AA588-589, and may additionally include a substitution of one or two amino acids at amino acid positions flanking the 7-mer sequence (e.g., AA587-588 and/or AA589-590) to produce an eleven amino acid polymer (11-mer) at the 588 loop of a parental AAV capsid protein. The first column of FIG. 1 lists 7mer AA insertion sequences at 588-589 along with the two flanking amino acids on each side of the insertion (corresponding to AA positions 587, 588, 589, and 590 of the parental capsid). The flanking amino acids may include substitutions. The second and third columns in FIG. 1 list the AA sequence at positions 452-458 (relative to the parental capsid) and the corresponding brain enrichment score respectively for each modified AAV capsid.

In some aspects, the insertion amino acid sequence is at least 71.4% identical to the amino acid sequence provided in Tables 1-3, FIG. 1 and/or Formula I. In some aspects, the insertion amino acid sequence is at least 86.7% identical to the amino acid sequence provided in Tables 1-3, FIG. 1 and/or Formula I.

Also disclosed herein are methods and kits for producing therapeutic recombinant AAV (rAAV) particles, as well as methods and pharmaceutical compositions or formulations comprising the rAAV particles, for the treatment of a disease or condition affecting the brain.

Disclosed herein are AAV capsids engineered with increased viral transduction in the brain. The AAV capsids can encapsidate a viral vector with a heterologous nucleic acid encoding, for example, a therapeutic gene expression product. Transduction of the heterologous nucleic acid in the brain can be achieved upon systemic delivery to a subject of the AAV capsid of the present disclosure encapsidating a heterologous nucleic acid. The AAV capsids disclosed herein are advantageous for many applications of gene therapy to treat human disease, including, but not limited to, disorders of the central nervous system.

The recombinant AAV vectors comprising a nucleic acid sequence encoding the AAV capsid proteins of the present disclosure as also provided herein. For example, the viral vectors of the present disclosure comprise a nucleic acid sequence comprising the AAV viral Cap (Capsid) encoding VP1, VP2, and VP3, at least one of which is modified to produce the AAV capsid proteins of the present disclosure. The recombinant AAV vector provided can be derived from an AAV serotype (e.g., AAV9) or a variant AAV serotype including an insertion of the present invention.

### AAV Capsids

Provided herein are modified adeno-associated (AAV) virus capsid compositions useful for integrating a transgene into a target cell or environment (in a subject when they are administered systemically to the subject.

An rAAV comprises an AAV capsid that can be engineered to encapsidate a heterologous nucleic acid (e.g., therapeutic nucleic acid, gene editing machinery). The AAV capsid is made up of three AAV capsid protein monomers, VP1, VP2, and VP3. Sixty copies of these three VP proteins interact in a 1:1:10 ratio to form the viral capsid. VP1 covers the whole of VP2 protein in addition to a ˜137 amino acid N-terminal region (VP1u), VP2 covers the whole of VP3 in addition to ˜65 amino acid N-terminal region (VP1/2 common region). The three capsid proteins share a conserved amino acid sequence of VP3, which in some cases is the region beginning at amino acid position 138 (e.g., AA139-736).

While not wishing to be bound by theory, it is understood that a parent AAV capsid sequence comprises a VP1 region. In certain embodiments, a parent AAV capsid sequence comprises a VP1, VP2 and/or VP3 region, or any combination thereof. A parent VP1 sequence may be considered synonymous with a parent AAV capsid sequence.

The AAV VP3 structure contains highly conserved regions that are common to all serotypes, a core eight-stranded β-barrel motif (βB-βI) and a small α-helix (αA). The loop regions inserted between the β-strands consist of the distinctive HI loop between β-strands H and I, the DE loop between β-strands D and E, and nine variable regions (VRs), which form the top of the loops. These VRs, such as the AA588 loop, are found on the capsid surface and can be associated with specific functional roles in the AAV life cycle including receptor binding, transduction and antigenic specificity.

In some aspects, the rAAV variant of the present invention comprises an AAV capsid protein having a peptide insertion at the residues corresponding to amino acids 588-589 of the AAV9 native sequence of SEQ ID NO: 1.

The AAV capsids comprise AAV capsid proteins (e.g., VP1, VP2, and VP3), each with an insertion, such as in the 588 loop of a parental AAV capsid protein structure (AAV9 VP1 numbering). The 588 loop contains the site of heparan sulfate binding of AAV2 and is amenable to peptide display. The only known receptors for AAV9 is N-linked terminal galactose and AAV receptor (AAVR), but many indications point toward there being others. Modifications to AAV9 588 loop are shown herein to confer an increased transgene transduction in target in vivo environments.

The present invention provides, in an aspect, a peptide insertion at the AAV 588 loop comprising or consisting of an amino-acid sequence set forth in any one of Tables 1-3, FIG. 1 and/or Formula I.

Disclosed herein are AAV capsids comprising AAV capsid proteins with an insertion at the 588 loop that confer a higher transduction in brain cell types (e.g., brain endothelial cells, neurons, astrocytes). In particular, the AAV capsid proteins disclosed herein enable rAAV-mediated transduction of a heterologous nucleic acid (e.g., transgene) in the brain of a subject. The AAV capsids of the present disclosure may be formulated as a pharmaceutical composition. In addition, the AAV capsids can be isolated and purified to be used for a variety of applications.

In some embodiments, the rAAV capsid of the present disclosure are generated using the methods disclosed herein. In some instances, the rAAV capsid is chimeric. In some instances, the rAAV, or variant AAV protein comprises therein, confer an increase in a localization of the rAAV within the target tissue, as compared to the parental AAV capsid or capsid protein.

### AAV Capsid Proteins

Disclosed herein are recombinant AAV (rAAV) capsids which comprise AAV capsid proteins that are engineered with a modified capsid protein (e.g., VP1, VP2, VP3). In some embodiments, the rAAV capsid proteins of the present disclosure are generated using the methods disclosed herein. In some embodiments, the AAV capsid proteins are used in the methods of delivering a therapeutic nucleic acid (e.g., a transgene) to a subject. In some instances, the rAAV capsid proteins have desired AAV expression rendering them particularly suitable for certain therapeutic applications, e.g., the treatment of a disease or disorder in a subject such as those disclosed herein.

The rAAV capsid proteins are engineered for optimized expression in the CNS, for example the brain, of a subject upon systemic administration of the rAAV to the subject. The rAAV capsid proteins are engineered to include the insertions provided in Tables 1-3, FIG. 1 and/or Formula I. The rAAV capsid proteins including the insertions provided in Tables 1-3, FIG. 1 and/or Formula I are engineered to achieve efficient transduction of an encapsidated transgene. In particular, the rAAV capsid proteins have increased expression in the brain of a subject.

The engineered AAV capsid proteins described herein have, in some cases, an insertion of an amino acid that is heterologous to the parental AAV capsid protein at amino acid positions in the 588 loop. In some embodiments, the amino acid is not endogenous to the parental AAV capsid protein at the amino acid position of the insertion. The amino acid may be a naturally occurring amino acid in the same or equivalent amino acid position as the insertion of the substitution in a different AAV capsid protein.

The 7-mers described herein were advantageously generated using polymerase chain reaction (PCR) with degenerate primers, where each of the seven amino acids is encoded by a deoxyribose nucleic acid (DNA) sequence N—N-K. “N” is any of the four DNA nucleotides and K is guanine (G) or thymine (T). This method of generating random 7-mer amino acid sequences enables 1.28 billion possible combinations at the protein level.

The rAAV capsid proteins of the present disclosure comprise an insertion of an amino acid in an amino acid sequence of an AAV capsid protein. The AAV capsid, from which an engineered AAV capsid protein of the present disclosure is produced, is referred to as a “parental” AAV capsid. The complete genome of AAV-1 is provided in GenBank Accession No. NC_002077; the complete genome of AAV-2 is provided in GenBank Accession No. NC_001401 and Srivastava et al., J. Virol., 45: 555-564 (1983); the complete genome of AAV-3 is provided in GenBank Accession No. NC_1829; the complete genome of AAV-4 is provided in GenBank Accession No. NC_001829; the AAV-5 genome is provided in GenBank Accession No. AF085716; the complete genome of AAV-6 is provided in GenBank Accession No. NC_001862; at least portions of AAV-7 and AAV-8 genomes are provided in GenBank Accession Nos. AX753246 and AX753249, respectively; the AAV-9 genome is provided in Gao et al., J. Virol., 78: 6381-6388 (2004); the AAV-10 genome is provided in Mol. Ther., 13(1): 67-76 (2006); the AAV-11 genome is provided in Virology, 330(2): 375-383 (2004); portions of the AAV-12 genome are provided in Genbank Accession No. DQ813647; portions of the AAV-13 genome are provided in Genbank Accession No. EU285562.

In some cases, the parental AAV is derived from an AAV with a serotype selected from AAV1, AAV2, AAV3, AAV4, AAV5, AAV6, AAV7, AAV8, AAV9, AAV10, AAV11 and AAV12. The AAV capsid protein that is “derived” from another may be a variant AAV capsid protein. A variant may include, for example, a heterologous amino acid in an amino acid sequence of the AAV capsid protein. The heterologous amino acid may be non-naturally occurring in the AAV capsid protein. The heterologous amino acid may be naturally occurring in a different AAV capsid protein. In some instances, the parental AAV capsid is described in US Pat Publication 2020/0165576 and U.S. Pat. App. Ser. No. 62/832,826 and PCT/US20/20778; the content of each of which is incorporated herein.

In some instances, the parental AAV is AAV9. In some instances, the amino acid sequence of the AAV9 capsid protein comprises SEQ ID NO: 1. The amino acid sequence of AAV9 VP1 capsid protein (>tr|Q6JC40|Q6JC40_9VIRU Capsid protein VP1 OS=Adeno-associated virus 9 OX=235455 GN=cap PE=1 SV=1) is provided in SEQ ID NO: 1 (MAADGYLPDWLEDNLSEGIREWWALKPGAPQPKANQQHQDNARGLVLPGYKYLGPG NGLDKGEPVNAADAAALEHDKAYDQQLKAGDNPYLKYNHADAEFQERLKEDTSFGG NLGRAVFQAKKRLLEPLGLVEEAAKTAPGKKRPVEQSPQEPDSSAGIGKSGAQPAKKRL NFGQTGDTESVPDPQPIGEPPAAPSGVGSLTMASGGGAPVADNNEGADGVGSSSGNWH CDSQWLGDRVITTSTRTWALPTYNNHLYKQISNSTSGGSSNDNAYFGYSTPWGYFDFN RFHCHFSPRDWQRLINNNWGFRPKRLNFKLFNIQVKEVTDNNGVKTIANNLTSTVQVFT DSDYQLPYVLGSAHEGCLPPFPADVFMIPQYGYLTLNDGSQAVGRSSFYCLEYFPSQML RTGNNFQFSYEFENVPFHSSYAHSQSLDRLMNPLIDQYLYYLSKTINGSGQNQQTLKFSV AGPSNMAVQGRNYIPGPSYRQQRVSTTVTQNNNSEFAWPGASSWALNGRNSLMNPGP AMASHKEGEDRFFPLSGSLIFGKQGTGRDNVDADKVMITNEEEIKTTNPVATESYGQVA TNHQSAQAQAQTGWVQNQGILPGMVWQDRDVYLQGPIWAKIPHTDGNFHPSPLMGGF GMKHPPPQILIKNTPVPADPPTAFNKDKLNSFITQYSTGQVSVEIEWELQKENSKRWNPE IQYTSNYYKSNNVEFAVNTEGVYSEPRPIGTRYLTRNL). In some instances, the parental AAV capsid protein sequence is 70%, 75%, 80%, 85%, 90%, 91%, 92%, 93%, 94%, 95%, 96%, 97%, 98%, 99%, or 100% homologous to SEQ ID NO: 1.

AAV capsid proteins from native AAV serotypes, such as AAV9, with tropisms including the liver activate the innate immune response, which in some cases causes a severe inflammatory response in a subject, which can lead to multi-organ failure. By improving transduction of a native AAV serotype for a target in vivo tissue (e.g., brain), and potentially decreased transduction in off target tissue (e.g., liver) the rAAV particles of the present disclosure reduce the immunogenic properties of AAV-mediated transgene delivery and prevent activation of the innate immune response.

In some instances, the parental AAV capsid protein comprises the entire VP1 region provided in SEQ ID NO: 1 (e.g., amino acids 1-736). In some instances, the parental AAV capsid protein comprises amino acids 217-736 in SEQ ID NO: 1, which is the common region found in VP1, VP2 and VP3 AAV9 capsid proteins. In some instances, the AAV capsid protein comprises amino acids 64-736 in SEQ ID NO: 1, which is the common region found in VP1 and VP2. The parental AAV capsid protein sequence may comprise amino acids selected from 1-736, 10-736, 20-736, 30-736, 40-736, 50-736, 60-736, 70-736, 80-736, 90-736, 100-736, 110-736, 120-736, 130-736, 140-736, 150-736, 160-736, 170-736, 180-736, 190-736, 200-736, 210-736, 220-736, 230-736, 240-736, 250-736, 260-736, 270-736, 280-736, 290-736, 300-736, 310-736, 320-736, 330-736, 340-736, 350-736, 360-736, 370-736, 380-736, 390-736, 400-736, 410-736, 420-736, 430-736, 440-736, and 450-736, from SEQ ID NO: 1. In some aspects, the rAAV variant comprises an AAV capsid protein comprising an amino acid sequence that is at least 98% identical to amino acid 217 to amino acid 736 of SEQ ID NO: 1. In some instances, the amino acid insertion is at a three (3)-fold axis of symmetry of a corresponding parental AAV capsid protein.

Disclosed herein are insertions of an amino acid sequence in an AAV capsid protein. Where the sequence numbering designation “588-589” is noted for AAV9, for example AAV VP1, the invention also includes insertions in similar locations in the other AAV serotypes. As used herein, “AA588-589” indicates that the insertion of the amino acid (or amino acid sequence) is immediately after an amino acid (AA) at position 588 and immediately before an AA at position 589 within an amino acid sequence of a parental AAV VP capsid protein (VP1 numbering). Amino acids 587-591 include a motif comprising “AQAQA” as set forth in SEQ ID NO: 1. Exemplary AAV capsid protein sequences are provided in Table 3. For example, SNTTKSV (SEQ ID NO: 631) is inserted at AA588-589 in an AAV9 capsid amino acid sequence along with an A589D substitution to provide variant A (SEQ ID NO: 640). It is envisioned that the sequences disclosed herein (Tables 1-3, FIG. 1 and Formula I) may be inserted at AA588-589 in an amino acid sequence of a parental AAV9 capsid protein or at AA587-590 (replacing amino acids AA587-590), a variant thereof, or equivalent amino acid position of a parental AAV of a different serotype (e.g., AAV1, AAV2, AAV3, and the like). In certain embodiments, the aforementioned “AQAQ” sequence flanking the insertion may include one or more substitutions. In any AAV capsid protein sequence disclosed herein, the amino acid at position 449 may be R or K.

TABLE 3
Exemplary AAV Capsid Protein Sequences

| SEQ ID NO: | Identifier | Sequence |
|---|---|---|
| 640 | Variant A | MAADGYLPDWLEDNLSEGIREWWALKPGAPQPKANQ |
| | | QHQDNARGLVLPGYKYLGPGNGLDKGEPVNAADAAA |
| | | LEHDKAYDQQLKAGDNPYLKYNHADAEFQERLKEDT |
| | | SFGGNLGRAVFQAKKRLLEPLGLVEEAAKTAPGKKRP |
| | | VEQSPQEPDSSAGIGKSGAQPAKKRLNFGQTGDTESVP |
| | | DPQPIGEPPAAPSGVGSLTMASGGGAPVADNNEGADG |
| | | VGSSSGNWHCDSQWLGDRVITTSTRTWALPTYNNHLY |
| | | KQISNSTSGGSSNDNAYFGYSTPWGYFDFNRFHCHFSP |
| | | RDWQRLINNNWGFRPKRLNFKLFNIQVKEVTDNNGVK |
| | | TIANNLTSTVQVFTDSDYQLPYVLGSAHEGCLPPFPAD |
| | | VFMIPQYGYLTLNDGSQAVGRSSFYCLEYFPSQMLRTG |
| | | NNFQFSYEFENVPFHSSYAHSQSLDRLMNPLIDQYLYY |
| | | LSRTINGSGQNQQTLKFSVAGPSNMAVQGRNYIPGPSY |
| | | RQQRVSTTVTQNNNSEFAWPGASSWALNGRNSLMNP |
| | | GPAMASHKEGEDRFFPLSGSLIFGKQGTGRDNVDADK |
| | | VMITNEEEIKTTNPVATESYGQVATNHQSAQSNTTKSV |
| | | DQAQTGWVQNQGILPGMVWQDRDVYLQGPIWAKIPH |
| | | TDGNFHPSPLMGGFGMKHPPPQILIKNTPVPADPPTAFN |
| | | KDKLNSFITQYSTGQVSVEIEWELQKENSKRWNPEIQY |
| | | TSNYYKSNNVEFAVNTEGVYSEPRPIGTRYLTRNL |
| 641 | Variant B | MAADGYLPDWLEDNLSEGIREWWALKPGAPQPKANQ |
| | | QHQDNARGLVLPGYKYLGPGNGLDKGEPVNAADAAA |
| | | LEHDKAYDQQLKAGDNPYLKYNHADAEFQERLKEDT |
| | | SFGGNLGRAVFQAKKRLLEPLGLVEEAAKTAPGKKRP |
| | | VEQSPQEPDSSAGIGKSGAQPAKKRLNFGQTGDTESVP |
| | | DPQPIGEPPAAPSGVGSLTMASGGGAPVADNNEGADG |
| | | VGSSSGNWHCDSQWLGDRVITTSTRTWALPTYNNHLY |
| | | KQISNSTSGGSSNDNAYFGYSTPWGYFDFNRFHCHFSP |
| | | RDWQRLINNNWGFRPKRLNFKLFNIQVKEVTDNNGVK |
| | | TIANNLTSTVQVFTDSDYQLPYVLGSAHEGCLPPFPAD |
| | | VFMIPQYGYLTLNDGSQAVGRSSFYCLEYFPSQMLRTG |
| | | NNFQFSYEFENVPFHSSYAHSQSLDRLMNPLIDQYLYY |
| | | LSRTINGSGQNQQTLKFSVAGPSNMAVQGRNYIPGPSY |
| | | RQQRVSTTVTQNNNSEFAWPGASSWALNGRNSLMNP |
| | | GPAMASHKEGEDRFFPLSGSLIFGKQGTGRDNVDADK |
| | | VMITNEEEIKTTNPVATESYGQVATNHQSAQQNTTKS |
| | | VDQAQTGWVQNQGILPGMVWQDRDVYLQGPIWAKIP |
| | | HTDGNFHPSPLMGGFGMKHPPPQILIKNTPVPADPPTAF |
| | | NKDKLNSFITQYSTGQVSVEIEWELQKENSKRWNPEIQ |
| | | YTSNYYKSNNVEFAVNTEGVYSEPRPIGTRYLTRNL |
| 642 | Variant C | MAADGYLPDWLEDNLSEGIREWWALKPGAPQPKANQ |
| | | QHQDNARGLVLPGYKYLGPGNGLDKGEPVNAADAAA |
| | | LEHDKAYDQQLKAGDNPYLKYNHADAEFQERLKEDT |
| | | SFGGNLGRAVFQAKKRLLEPLGLVEEAAKTAPGKKRP |
| | | VEQSPQEPDSSAGIGKSGAQPAKKRLNFGQTGDTESVP |
| | | DPQPIGEPPAAPSGVGSLTMASGGGAPVADNNEGADG |
| | | VGSSSGNWHCDSQWLGDRVITTSTRTWALPTYNNHLY |
| | | KQISNSTSGGSSNDNAYFGYSTPWGYFDFNRFHCHFSP |
| | | RDWQRLINNNWGFRPKRLNFKLFNIQVKEVTDNNGVK |
| | | TIANNLTSTVQVFTDSDYQLPYVLGSAHEGCLPPFPAD |
| | | VFMIPQYGYLTLNDGSQAVGRSSFYCLEYFPSQMLRTG |
| | | NNFQFSYEFENVPFHSSYAHSQSLDRLMNPLIDQYLYY |
| | | LSRTINGSGQNQQTLKFSVAGPSNMAVQGRNYIPGPSY |
| | | RQQRVSTTVTQNNNSEFAWPGASSWALNGRNSLMNP |
| | | GPAMASHKEGEDRFFPLSGSLIFGKQGTGRDNVDADK |
| | | VMITNEEEIKTTNPVATESYGQVATNHQS |
| | | ADLNTTKPVMQAQTGWVQNQGILPGMVWQDRDVYL |
| | | QGPIWAKIPHTDGNFHPSPLMGGFGMKHPPPQILIKNTP |
| | | VPADPPTAFNKDKLNSFITQYSTGQVSVEIEWELQKEN |
| | | SKRWNPEIQYTSNYYKSNNVEFAVNTEGVYSEPRPIGT |
| | | RYLTRNL |
| 643 | Variant D | MAADGYLPDWLEDNLSEGIREWWALKPGAPQPKANQ |
| | | QHQDNARGLVLPGYKYLGPGNGLDKGEPVNAADAAA |
| | | LEHDKAYDQQLKAGDNPYLKYNHADAEFQERLKEDT |
| | | SFGGNLGRAVFQAKKRLLEPLGLVEEAAKTAPGKKRP |
| | | VEQSPQEPDSSAGIGKSGAQPAKKRLNFGQTGDTESVP |
| | | DPQPIGEPPAAPSGVGSLTMASGGGAPVADNNEGADG |
| | | VGSSSGNWHCDSQWLGDRVITTSTRTWALPTYNNHLY |
| | | KQISNSTSGGSSNDNAYFGYSTPWGYFDFNRFHCHFSP |
| | | RDWQRLINNNWGFRPKRLNFKLFNIQVKEVTDNNGVK |
| | | TIANNLTSTVQVFTDSDYQLPYVLGSAHEGCLPPFPAD |
| | | VFMIPQYGYLTLNDGSQAVGRSSFYCLEYFPSQMLRTG |
| | | NNFQFSYEFENVPFHSSYAHSQSLDRLMNPLIDQYLYY |
| | | LSRTINGSGQNQQTLKFSVAGPSNMAVQGRNYIPGPSY |
| | | RQQRVSTTVTQNNNSEFAWPGASSWALNGRNSLMNP |
| | | GPAMASHKEGEDRFFPLSGSLIFGKQGTGRDNVDADK |
| | | VMITNEEEIKTTNPVATESYGQVATNHQSAQANTTKS |
| | | VDQAQTGWVQNQGILPGMVWQDRDVYLQGPIWAKIP |
| | | HTDGNFHPSPLMGGFGMKHPPPQILIKNTPVPADPPTAF |
| | | NKDKLNSFITQYSTGQVSVEIEWELQKENSKRWNPEIQ |
| | | YTSNYYKSNNVEFAVNTEGVYSEPRPIGTRYLTRNL |
| 644 | Variant E | MAADGYLPDWLEDNLSEGIREWWALKPGAPQPKANQ |
| | | QHQDNARGLVLPGYKYLGPGNGLDKGEPVNAADAAA |
| | | LEHDKAYDQQLKAGDNPYLKYNHADAEFQERLKEDT |
| | | SFGGNLGRAVFQAKKRLLEPLGLVEEAAKTAPGKKRP |
| | | VEQSPQEPDSSAGIGKSGAQPAKKRLNFGQTGDTESVP |
| | | DPQPIGEPPAAPSGVGSLTMASGGGAPVADNNEGADG |
| | | VGSSSGNWHCDSQWLGDRVITTSTRTWALPTYNNHLY |
| | | KQISNSTSGGSSNDNAYFGYSTPWGYFDFNRFHCHFSP |
| | | RDWQRLINNNWGFRPKRLNFKLFNIQVKEVTDNNGVK |
| | | TIANNLTSTVQVFTDSDYQLPYVLGSAHEGCLPPFPAD |
| | | VFMIPQYGYLTLNDGSQAVGRSSFYCLEYFPSQMLRTG |
| | | NNFQFSYEFENVPFHSSYAHSQSLDRLMNPLIDQYLYY |
| | | LSRTINGSGQNQQTLKFSVAGPSNMAVQGRNYIPGPSY |
| | | RQQRVSTTVTQNNNSEFAWPGASSWALNGRNSLMNP |
| | | GPAMASHKEGEDRFFPLSGSLIFGKQGTGRDNVDADK |
| | | VMITNEEEIKTTNPVATESYGQVATNHQSAQNNTTKS |
| | | VDQAQTGWVQNQGILPGMVWQDRDVYLQGPIWAKIP |
| | | HTDGNFHPSPLMGGFGMKHPPPQILIKNTPVPADPPTAF |
| | | NKDKLNSFITQYSTGQVSVEIEWELQKENSKRWNPEIQ |
| | | YTSNYYKSNNVEFAVNTEGVYSEPRPIGTRYLTRNL |
| 645 | Variant F | MAADGYLPDWLEDNLSEGIREWWALKPGAPQPKANQ |
| | | QHQDNARGLVLPGYKYLGPGNGLDKGEPVNAADAAA |
| | | LEHDKAYDQQLKAGDNPYLKYNHADAEFQERLKEDT |
| | | SFGGNLGRAVFQAKKRLLEPLGLVEEAAKTAPGKKRP |
| | | VEQSPQEPDSSAGIGKSGAQPAKKRLNFGQTGDTESVP |
| | | DPQPIGEPPAAPSGVGSLTMASGGGAPVADNNEGADG |
| | | VGSSSGNWHCDSQWLGDRVITTSTRTWALPTYNNHLY |
| | | KQISNSTSGGSSNDNAYFGYSTPWGYFDFNRFHCHFSP |
| | | RDWQRLINNNWGFRPKRLNFKLFNIQVKEVTDNNGVK |
| | | TIANNLTSTVQVFTDSDYQLPYVLGSAHEGCLPPFPAD |
| | | VFMIPQYGYLTLNDGSQAVGRSSFYCLEYFPSQMLRTG |
| | | NNFQFSYEFENVPFHSSYAHSQSLDRLMNPLIDQYLYY |
| | | LSRTINGSGQNQQTLKFSVAGPSNMAVQGRNYIPGPSY |
| | | RQQRVSTTVTQNNNSEFAWPGASSWALNGRNSLMNP |
| | | GPAMASHKEGEDRFFPLSGSLIFGKQGTGRDNVDADK |
| | | VMITNEEEIKTTNPVATESYGQVATNHQSAQLNTTKN |
| | | VDQAQTGWVQNQGILPGMVWQDRDVYLQGPIWAKIP |
| | | HTDGNFHPSPLMGGFGMKHPPPQILIKNTPVPADPPTAF |
| | | NKDKLNSFITQYSTGQVSVEIEWELQKENSKRWNPEIQ |
| | | YTSNYYKSNNVEFAVNTEGVYSEPRPIGTRYLTRNL |
| 646 | Variant G | MAADGYLPDWLEDNLSEGIREWWALKPGAPQPKANQ |
| | | QHQDNARGLVLPGYKYLGPGNGLDKGEPVNAADAAA |
| | | LEHDKAYDQQLKAGDNPYLKYNHADAEFQERLKEDT |
| | | SFGGNLGRAVFQAKKRLLEPLGLVEEAAKTAPGKKRP |
| | | VEQSPQEPDSSAGIGKSGAQPAKKRLNFGQTGDTESVP |
| | | DPQPIGEPPAAPSGVGSLTMASGGGAPVADNNEGADG |
| | | VGSSSGNWHCDSQWLGDRVITTSTRTWALPTYNNHLY |
| | | KQISNSTSGGSSNDNAYFGYSTPWGYFDFNRFHCHFSP |
| | | RDWQRLINNNWGFRPKRLNFKLFNIQVKEVTDNNGVK |
| | | TIANNLTSTVQVFTDSDYQLPYVLGSAHEGCLPPFPAD |
| | | VFMIPQYGYLTLNDGSQAVGRSSFYCLEYFPSQMLRTG |
| | | NNFQFSYEFENVPFHSSYAHSQSLDRLMNPLIDQYLYY |
| | | LSRTINGSGQNQQTLKFSVAGPSNMAVQGRNYIPGPSY |
| | | RQQRVSTTVTQNNNSEFAWPGASSWALNGRNSLMNP |
| | | GPAMASHKEGEDRFFPLSGSLIFGKQGTGRDNVDADK |
| | | VMITNEEEIKTTNPVATESYGQVATNHQSADLNTTKS |
| | | VAQAQTGWVQNQGILPGMVWQDRDVYLQGPIWAKIP |
| | | HTDGNFHPSPLMGGFGMKHPPPQILIKNTPVPADPPTAF |
| | | NKDKLNSFITQYSTGQVSVEIEWELQKENSKRWNPEIQ |
| | | YTSNYYKSNNVEFAVNTEGVYSEPRPIGTRYLTRNL |
| 647 | Variant H | MAADGYLPDWLEDNLSEGIREWWALKPGAPQPKANQ |
| | | QHQDNARGLVLPGYKYLGPGNGLDKGEPVNAADAAA |
| | | LEHDKAYDQQLKAGDNPYLKYNHADAEFQERLKEDT |
| | | SFGGNLGRAVFQAKKRLLEPLGLVEEAAKTAPGKKRP |
| | | VEQSPQEPDSSAGIGKSGAQPAKKRLNFGQTGDTESVP |
| | | DPQPIGEPPAAPSGVGSLTMASGGGAPVADNNEGADG |
| | | VGSSSGNWHCDSQWLGDRVITTSTRTWALPTYNNHLY |
| | | KQISNSTSGGSSNDNAYFGYSTPWGYFDFNRFHCHFSP |
| | | RDWQRLINNNWGFRPKRLNFKLFNIQVKEVTDNNGVK |
| | | TIANNLTSTVQVFTDSDYQLPYVLGSAHEGCLPPFPAD |
| | | VFMIPQYGYLTLNDGSQAVGRSSFYCLEYFPSQMLRTG |
| | | NNFQFSYEFENVPFHSSYAHSQSLDRLMNPLIDQYLYY |
| | | LSRTINGSGQNQQTLKFSVAGPSNMAVQGRNYIPGPSY |
| | | RQQRVSTTVTQNNNSEFAWPGASSWALNGRNSLMNP |
| | | GPAMASHKEGEDRFFPLSGSLIFGKQGTGRDNVDADK |
| | | VMITNEEEIKTTNPVATESYGQVATNHQSAQDNTTKS |
| | | VFQAQTGWVQNQGILPGMVWQDRDVYLQGPIWAKIP |
| | | HTDGNFHPSPLMGGFGMKHPPPQILIKNTPVPADPPTAF |
| | | NKDKLNSFITQYSTGQVSVEIEWELQKENSKRWNPEIQ |
| | | YTSNYYKSNNVEFAVNTEGVYSEPRPIGTRYLTRNL |
| 648 | Variant I | MAADGYLPDWLEDNLSEGIREWWALKPGAPQPKANQ |
| | | QHQDNARGLVLPGYKYLGPGNGLDKGEPVNAADAAA |
| | | LEHDKAYDQQLKAGDNPYLKYNHADAEFQERLKEDT |
| | | SFGGNLGRAVFQAKKRLLEPLGLVEEAAKTAPGKKRP |
| | | VEQSPQEPDSSAGIGKSGAQPAKKRLNFGQTGDTESVP |
| | | DPQPIGEPPAAPSGVGSLTMASGGGAPVADNNEGADG |
| | | VGSSSGNWHCDSQWLGDRVITTSTRTWALPTYNNHLY |
| | | KQISNSTSGGSSNDNAYFGYSTPWGYFDFNRFHCHFSP |
| | | RDWQRLINNNWGFRPKRLNFKLFNIQVKEVTDNNGVK |
| | | TIANNLTSTVQVFTDSDYQLPYVLGSAHEGCLPPFPAD |
| | | VFMIPQYGYLTLNDGSQAVGRSSFYCLEYFPSQMLRTG |
| | | NNFQFSYEFENVPFHSSYAHSQSLDRLMNPLIDQYLYY |
| | | LSRTINGSGQNQQTLKFSVAGPSNMAVQGRNYIPGPSY |
| | | RQQRVSTTVTQNNNSEFAWPGASSWALNGRNSLMNP |
| | | GPAMASHKEGEDRFFPLSGSLIFGKQGTGRDNVDADK |
| | | VMITNEEEIKTTNPVATESYGQVATNHQSADLNTTKS |
| | | VSQAQTGWVQNQGILPGMVWQDRDVYLQGPIWAKIP |
| | | HTDGNFHPSPLMGGFGMKHPPPQILIKNTPVPADPPTAF |
| | | NKDKLNSFITQYSTGQVSVEIEWELQKENSKRWNPEIQ |
| | | YTSNYYKSNNVEFAVNTEGVYSEPRPIGTRYLTRNL |
| 649 | Variant J | MAADGYLPDWLEDNLSEGIREWWALKPGAPQPKANQ |
| | | QHQDNARGLVLPGYKYLGPGNGLDKGEPVNAADAAA |
| | | LEHDKAYDQQLKAGDNPYLKYNHADAEFQERLKEDT |
| | | SFGGNLGRAVFQAKKRLLEPLGLVEEAAKTAPGKKRP |
| | | VEQSPQEPDSSAGIGKSGAQPAKKRLNFGQTGDTESVP |
| | | DPQPIGEPPAAPSGVGSLTMASGGGAPVADNNEGADG |
| | | VGSSSGNWHCDSQWLGDRVITTSTRTWALPTYNNHLY |
| | | KQISNSTSGGSSNDNAYFGYSTPWGYFDFNRFHCHFSP |
| | | RDWQRLINNNWGFRPKRLNFKLFNIQVKEVTDNNGVK |
| | | TIANNLTSTVQVFTDSDYQLPYVLGSAHEGCLPPFPAD |
| | | VFMIPQYGYLTLNDGSQAVGRSSFYCLEYFPSQMLRTG |
| | | NNFQFSYEFENVPFHSSYAHSQSLDRLMNPLIDQYLYY |
| | | LSRTINGSGQNQQTLKFSVAGPSNMAVQGRNYIPGPSY |
| | | RQQRVSTTVTQNNNSEFAWPGASSWALNGRNSLMNP |
| | | GPAMASHKEGEDRFFPLSGSLIFGKQGTGRDNVDADK |
| | | VMITNEEEIKTTNPVATESYGQVATNHQSAQGNTTKS |
| | | VDQAQTGWVQNQGILPGMVWQDRDVYLQGPIWAKIP |
| | | HTDGNFHPSPLMGGFGMKHPPPQILIKNTPVPADPPTAF |
| | | NKDKLNSFITQYSTGQVSVEIEWELQKENSKRWNPEIQ |
| | | YTSNYYKSNNVEFAVNTEGVYSEPRPIGTRYLTRNL |

The insertions described herein may, in some cases, comprise a 7-mer insertion at AA588-589. It is envisioned that any 7-mer insertion disclosed herein in addition to a substitution with any amino acid at amino acid positions 587-590 [AQAQ] may comprise an 11-mer.

Disclosed herein are AAV capsid proteins with an insertion described above in a parental AAV capsid protein that confers an increased transduction in the brain in a subject, even when delivered systemically. One of the many advantages of the AAV capsid proteins described herein is their ability to target tissue and cells within the brain. The tissue can be the brain. Non-limiting examples of brain cells include a neuron and a glial cell. Glial cells can be selected from an oligodendrocyte, an ependymal cell, an astrocyte and a microglia. Another advantage among certain AAV capsid proteins described herein is a detargeting effect with respect to liver tissue relative to the parental AAV capsid protein.

In some instances, the AAV capsid protein comprises an insertion/substitution of at least or about seven, eight, nine, ten or eleven amino acids of an amino acid sequence of Tables 1-3, FIG. 1 and Formula I at an amino acid position 588-589 or at 587-590 in a parental AAV9 capsid protein (SEQ ID NO: 1). In some cases, the AAV capsid protein has an increased viral transduction enrichment in brain. In some cases, the AAV capsid protein has an decreased viral transduction enrichment in liver.

The rAAV capsid proteins of the present disclosure may also have a substitution of an amino acid sequence at amino acid position 452-458 in a parental AAV9 capsid protein, or variant thereof, as described in WO2020068990. Exemplary substitutions at amino acid position 452-458 in the parental AAV9 capsid protein can be found in the second column of FIG. 1.

The rAAV capsid proteins described herein may be isolated and purified. The AAV may be isolated and purified by methods standard in the art such as by column chromatography, iodixanol gradients, or cesium chloride gradients. Methods for purifying AAV from helper virus are known in the art and may include methods disclosed in, for example, Clark et al., Hum. Gene Ther., 10(6): 1031-1039 (1999); Schenpp and Clark, Methods Mol. Med., 69: 427-443 (2002); U.S. Pat. No. 6,566,118 and WO 98/09657.

In addition, the AAV capsid proteins disclosed herein, either isolated and purified, or not, may be formulated into a pharmaceutical formulation, which in some cases, further comprises a pharmaceutically acceptable carrier.

The rAAV capsid protein can be conjugated to a nanoparticle, a second molecule, or a viral capsid protein. In some cases, the nanoparticle or viral capsid protein would encapsidate the therapeutic nucleic acid described herein. In some instances, the second molecule is a therapeutic agent, e.g., a small molecule, antibody, antigen-binding fragment, peptide, or protein, such as those described herein.

“Percent Identity” is the percent of the symbols that actually match. Percent Similarity is the percent of the symbols that are similar. Symbols that are across from gaps are ignored. A similarity is scored when the scoring matrix value for a pair of symbols is greater than or equal to 0.50, the similarity threshold. The scoring matrix used in Version 10 of the Wisconsin Genetics Software Package is BLOSUM62 (see: Henikoff and Henikoff, (1989) Proc. Natl. Acad. Sci. USA 89: 10915).

Sequence identity/similarity values provided herein can refer to the value obtained using the BLAST+2.5.0 suite of programs using default settings (blast.ncbi.nlm.nih.gov) (Camacho, C., et al. (2009) BLAST+: architecture and applications. BMC Bioinformatics 10:421).

As those of ordinary skill in the art will understand, BLAST searches assume that proteins can be modeled as random sequences. However, many real proteins comprise regions of nonrandom sequences, which may be homopolymeric tracts, short-period repeats, or regions enriched in one or more amino acids. Such low-complexity regions may be aligned between unrelated proteins even though other regions of the protein are entirely dissimilar. A number of low-complexity filter programs can be employed to reduce such low-complexity alignments. For example, the SEG (Wooten and Federhen, (1993) Comput. Chem. 17: 149-63) and XNU (Ci-ayerie and States (1993) Comput. Chem. 17: 191-201) low-complexity filters can be employed alone or in combination.

The terms “substantial identity” and “substantially identical” indicate that a polypeptide or nucleic acid comprises a sequence with between 55-100% sequence identity to a reference sequence, with at least 55% sequence identity, or at least 60%, or at least 65%, or at least 70%, or at least 75%, or at least 80%, or at least 85%, or at least 90%, or at least 95%, or at least 99% sequence identity or any percentage of value within the range of 55-100% sequence identity relative to the reference sequence. The percent sequence identity may occur over a specified comparison window. Optimal alignment may be ascertained or conducted using the homology alignment algorithm of Needleman and Wunsch, supra.

For example, the insertion sequences may include, but are not limited to, sequences that are not exactly the same as the sequences disclosed herein, but which have, in addition to the substitutions explicitly described for various sequences listed herein, additional substitutions of amino acid residues which substantially do not impair the activity or properties of the sequences described herein, such as those predicted by homology software e.g. BLOSUM62 matrices.

### AAV Particles

The rAAV particles with the insertion sequences described herein have an increased transduction enrichment in the brain. In some instances, the increased transduction enrichment comprises a 1-fold, 2-fold, 3-fold, 4-fold, 5-fold, 6-fold, 7-fold, 8-fold, 9-fold or 10-fold increase, or more. In some instances, the increased transduction enrichment is at least 1-fold. In some instances, the increased transduction enrichment is at least 2-fold. In some instances, the increased transduction enrichment is at least 4-fold.

The rAAV particles with the insertion sequences described herein have an increased expression enrichment in the brian. Detecting whether a rAAV possesses more or less specificity for a target in vivo environment, includes measuring a level of gene expression product (e.g., RNA or protein) expressed from the heterologous nucleic acid encapsidated by the rAAV in a tissue sample obtained from a subject. Suitable methods for measuring expression of a gene expression product include next-generation sequencing (NGS) and quantitative polymerase chain reaction (qPCR).

The increased expression in the brain is represented by the enrichment values provided in FIG. 1.

### Heterologous Nucleic Acids

Disclosed herein are therapeutic nucleic acids useful for the treatment or prevention of a disease or condition, or symptom of the disease or condition. In some embodiments, the therapeutic nucleic acids encode a therapeutic gene expression product. Non-limiting examples of gene expression products include proteins, polypeptides, peptides, enzymes, antibodies, antigen binding fragments, nucleic acid (RNA, DNA, antisense oligonucleotide, siRNA, and the like), and gene editing components, for use in the treatment, prophylaxis, and/or amelioration of the disease or disorder, or symptoms of the disease or disorder. In some instances, the therapeutic nucleic acids are placed in an organism, cell, tissue or organ of a subject by way of a rAAV, such as those disclosed herein.

Disclosed herein are rAAVs, each comprising a viral vector (e.g., a single stranded DNA molecule (ssDNA)). In some instances, the viral vector comprises two inverted terminal repeat (ITR) sequences that are about 145 bases each, flanking a transgene. In some embodiments, the transgene comprises a therapeutic nucleic acid, and in some cases, a promoter in cis with the therapeutic nucleic acid in an open reading frame (ORF). The promoter is capable of initiating transcription of therapeutic nucleic acid in the nucleus of the target cell. The ITR sequences can be from any AAV serotype. Non-limiting examples of AAV serotypes include AAV1, AAV2, AAV3, AAV4, AAV5, AAV6, AAV7, AAV8, AAV9, AAV10, AAV11, and AAV12. In some cases, an ITR is from AAV2. In some cases, an ITR is from AAV9.

Disclosed herein are transgenes that can comprise any number of nucleotides. In some cases, a transgene can comprise less than about 100 nucleotides. In some cases, a transgene can comprise at least about 100 nucleotides. In some cases, a transgene can comprise at least about 200 nucleotides. In some cases, a transgene can comprise at least about 300 nucleotides. In some cases, a transgene can comprise at least about 400 nucleotides. In some cases, a transgene can comprise at least about 500 nucleotides. In some cases, a transgene can comprise at least about 1000 nucleotides. In some cases, a transgene can comprise at least about 5000 nucleotides. In some cases, a transgene can comprise over 5,000 nucleotides. In some cases, a transgene can comprise between about 500 and about 5000 nucleotides. In some cases, a transgene comprises about 5000 nucleotides. In any of the cases disclosed herein, the transgene can comprise DNA, RNA, or a hybrid of DNA and RNA. In some cases, the transgene can be single stranded. In some cases, the transgene can be double stranded.

Disclosed herein are transgenes useful for modulating the expression or activity of a target gene or gene expression product thereof. In some instances, the transgene is encapsidated by an rAAV capsid protein of an rAAV particle described herein. In some instances, the rAAV particle is delivered to a subject to treat a disease or condition disclosed herein in the subject. In some instances, the delivery is systemic.

The transgenes disclosed herein are useful for expressing an endogenous gene at a level similar to that of a healthy or normal individual. This is particularly useful in the treatment of a disease or condition related to the underexpression, or lack of expression, of a gene expression product. In some embodiments, the transgenes disclosed herein are useful for overexpressing an endogenous gene, such that an expression level of the endogenous gene is above the expression level of a healthy or normal individual. Additionally, transgenes can be used to express exogenous genes (e.g., active agent such as an antibody, peptide, nucleic acid, or gene editing components). In some embodiments, the therapeutic gene expression product is capable of altering, enhancing, increasing, or inducing the activity of one or more endogenous biological processes in the cell. In some embodiments, the transgenes disclosed herein are useful for reducing expression of an endogenous gene, for example, a dominant negative gene. In some embodiments, the therapeutic gene expression product is capable of altering, inhibiting, reducing, preventing, eliminating, or impairing the activity of one or more endogenous biological processes in the cell. In some aspects, the increase of gene expression refers to an increase by at least about 20%, 30%, 40%, 50%, 60%, 70%, 80%, 85%, 90%, 95% and 100%. In certain aspects, the protein product of the targeted gene may be increased by at least about 20%, 30%, 40%, 50%, 60%, 70%, 80%, 85%, 90%, 95% and 100%. In some aspects, the decrease of gene expression refers to an increase by at least about 20%, 30%, 40%, 50%, 60%, 70%, 80%, 85%, 90%, 95% and 100%. In certain aspects, the protein product of the targeted gene may be decreased by at least about 20%, 30%, 40%, 50%, 60%, 70%, 80%, 85%, 90%, 95% and 100%.

When endogenous sequences (endogenous or part of a transgene) are expressed with a transgene, the endogenous sequences can be full-length sequences (wild-type or mutant) or partial sequences. The endogenous sequences can be functional. Non-limiting examples of the function of these full length or partial sequences include increasing the serum half-life of the polypeptide expressed by a transgene (e.g., therapeutic gene) and/or acting as a carrier.

A transgene can be inserted into an endogenous gene such that all, some or none of the endogenous gene is expressed. For example, a transgene as described herein can be inserted into an endogenous locus such that some (N-terminal and/or C-terminal to a transgene) or none of the endogenous sequences are expressed, for example as a fusion with a transgene. In other cases, a transgene (e.g., with or without additional coding sequences of the endogenous gene) is integrated into any endogenous locus, for example a safe-harbor locus. For example, a Frataxin (FXN) transgene can be inserted into an endogenous FXN gene. A transgene can be inserted into any gene, e.g., the genes as described herein.

At least one advantage of the present disclosure is that virtually any therapeutic nucleic acid may be used to express any therapeutic gene expression product. In some instances, the therapeutic gene expression product is a therapeutic protein or a peptide (e.g., antibody, antigen-binding fragment, peptide, or protein). In one embodiment the protein encoded by the therapeutic nucleic acid is between 50-5000 amino acids in length. In some embodiments the protein encoded is between 50-2000 amino acids in length. In some embodiments the protein encoded is between 50-1000 amino acids in length. In some embodiments the protein encoded is between 50-1500 amino acids in length. In some embodiments the protein encoded is between 50-800 amino acids in length. In some embodiments the protein encoded is between 50-600 amino acids in length. In some embodiments the protein encoded is between 50-400 amino acids in length. In some embodiments the protein encoded is between 50-200 amino acids in length. In some embodiments the protein encoded is between 50-100 amino acids in length. In some embodiments the peptide encoded is between 4-50 amino acids in length. In some embodiments, the protein encoded is a tetrapeptide, a pentapeptide, a hexapeptide, a heptapeptide, an octapeptide, a nonapeptide, or a decapeptide. In some embodiments, the protein encoded comprises a peptide of 2-30 amino acids, such as for example 5-30, 10-30, 2-25, 5-25, 10-25, or 10-20 amino acids. In some embodiments, the protein encoded comprises a peptide of at least 11, 12, 13, 14, 15, 17, 20, 25 or 30 amino acids, or a peptide that is no longer than 50 amino acids, e.g. no longer than 35, 30, 25, 20, 17, 15, 14, 13, 12, 11 or 10 amino acids.

Non-limiting examples of therapeutic protein or peptides include an adrenergic agonist, an anti-apoptosis factor, an apoptosis inhibitor, a cytokine receptor, a cytokine, a cytotoxin, an erythropoietic agent, a glutamic acid decarboxylase, a glycoprotein, a growth factor, a growth factor receptor, a hormone, a hormone receptor, an interferon, an interleukin, an interleukin receptor, a kinase, a kinase inhibitor, a nerve growth factor, a netrin, a neuroactive peptide, a neuroactive peptide receptor, a neurogenic factor, a neurogenic factor receptor, a neuropilin, a neurotrophic factor, a neurotrophin, a neurotrophin receptor, an N-methyl-D-aspartate antagonist, a plexin, a protease, a protease inhibitor, a protein decarboxylase, a protein kinase, a protein kinsase inhibitor, a proteolytic protein, a proteolytic protein inhibitor, a semaphoring, a semaphorin receptor, a serotonin transport protein, a serotonin uptake inhibitor, a serotonin receptor, a serpin, a serpin receptor, and a tumor suppressor. In certain embodiments, the therapeutic protein or peptide is selected from brain-derived neurotrophic factor (BDNF), ciliary neurotrophic factor (CNTF), macrophage colony-stimulating factor (CSF), epidermal growth factor (EGF), fibroblast growth factor (FGF), gonadotropin, interferon-gamma (IFN), insulin-like growth factor 1 (IFG-1), nerve growth factor (NGF), platelet-derived growth factor (PDGF), pigment epithelium-derived factor (PEDF), transforming growth factor (TGF), transforming growth factor-beta (TGF-B), tumor necrosis factor (TNF), vascular endothelial growth factor (VEGF), prolactin, somatotropin, X-linked inhibitor of apoptosis protein 1 (XIAP1), interleukin 1 (IL-1), IL-2, IL-3, IL-4, IL-5, IL-6, IL-7, IL-8, IL-9, IL-10, IL-10, viral IL-10, IL-11, IL-12, IL-13, IL-14, IL-15, IL-16, IL-17, and IL-18.

A therapeutic gene expression product can comprise gene editing components. Non-limiting examples of gene editing components include those required for CRISPR/Cas, artificial site-specific RNA endonuclease (ASRE), zinc finger endonuclease (ZFN), and transcription factor like effector nuclease (TALEN). In a non-limiting example, a subject having Huntington's disease is identified. The subject is then systemically administered a first amount of a rAAV encapsidating a viral vector encoding ZFN engineered to represses the transcription of the Huntingtin (HTT) gene. The rAAV will include a modified AAV capsid protein that includes an amino acid sequence provided in any one of Tables 1-3, FIG. 1, and Formula I, so as to allow proper targeting of the ZFN to the nervous system, while reducing expression in off-target organs, such as the liver. If needed, the subject is administered a second or third dose of the rAAV, until a therapeutically effective amount of the ZFN is expressed in the subject's nervous system.

A therapeutic nucleic acid can comprise a non-protein coding gene e.g., sequences encoding antisense RNAs, RNAi, shRNAs and micro RNAs (miRNAs), miRNA sponges or decoys, recombinase delivery for conditional gene deletion, conditional (recombinase-dependent) expression, includes those required for the gene editing components described herein. The non-protein coding gene may also encode a tRNA, rRNA, tmRNA, piRNA, double stranded RNA, snRNA, snoRNA, and/or long non-coding RNA (lncRNA). In some cases, the non-protein coding gene can modulate the expression or the activity of a target gene or gene expression product. For example, the RNAs described herein may be used to inhibit gene expression in the brain. In some cases, inhibition of gene expression refers to an inhibition by at least about 20%, 30%, 40%, 50%, 60%, 70%, 80%, 85%, 90%, 95% and 100%. In some cases, the protein product of the targeted gene may be inhibited by at least about 20%, 30%, 40%, 50%, 60%, 70%, 80%, 85%, 90%, 95% and 100%. The gene can be either a wild type gene or a gene with at least one mutation. The targeted protein may be either a wild type protein or a protein with at least one mutation.

A therapeutic nucleic acid can modulate the expression or activity of a gene or gene expression product expressed from the gene that is implicated in a disease or disorder of the brain. For example, the therapeutic nucleic acid, in some cases is a gene or a modified version of the gene described herein. In some instances, the gene or gene expression product is inhibited. In some instances, the gene or gene expression product is enhanced.

In another example, the therapeutic nucleic acid comprises an effector gene expression product such as a gene editing component specific to target a gene therein. Non-limited examples of genes include target gene or gene expression product selected from ATP1A2, CACNAIA, SETD5, SHANK3, NF2, DNMT1, TCF4, RAI1, PEX1, ARSA, EIF2B5, EIF2B1, EIF2B2, NPC1, ADAR, MFSD8, STXBP1, PRICKLE2, PRRT2, IDUA, STX1B, Sarcoglycan Alpha (SGCA), glutamic acid decarboxylase 65 (GAD65), glutamic acid decarboxylase 67 (GAD67), CLN2, Nerve Growth Factor (NGF), glial cell derived neurotrophic factor (GDNF), Survival Of Motor Neuron 1, STXBP1, Telomeric (SMN1), Factor X (FIX), Retinoid Isomerohydrolase (RPE65), sarco/endoplasmic reticulum Ca2+-ATPase (SERCA2a), Glucocerebrosidase (GCase), galactocerebrosidase (GALC), CDKL5, Frataxin (FXN), Huntingtin (HTT), methyl-CpG binding protein 2 (MECP2), a peroxisomal biogenesis factor (PEX), progranulin (GRN), an antitubulin agent, copper-zinc superoxide dismutase (SOD1), iduronate 2 sulfatase (hIDS), Glucosylceramidase Beta (GBA), fragile X mental retardation 1 (FMR1), NPC Intracellular Cholesterol Transporter 1 (NPC1), SCN1A, C9orf72, NPS3 and a NLRP3 inflammasome. In some embodiments, the peroxisomal biogenesis factor (PEX) is selected from PEX1, PEX2, PEX3, PEX4, PEX5, PEX6, PEX7, PEX10, PEX11β, PEX12, PEX13, PEX14, PEX16, PEX19, and PEX26. In some instances, the gene or gene expression product is inhibited. In some instances, the gene or gene expression product is enhanced.

### AAV Vectors

Aspects disclosed herein comprise plasmid vectors comprising a nucleic acid sequence encoding the AAV capsids and AAV capsid proteins described herein. AAV vectors described herein are useful for the assembly of a rAAV and viral packaging of a heterologous nucleic acid. In addition, an AAV vector may encode a transgene comprising the heterologous nucleic acid.

An AAV vector can comprise a transgene, which in some cases encodes a heterologous gene expression product (e.g., therapeutic gene expression product, recombinant capsid protein, and the like). The transgene is in cis with two inverted terminal repeats (ITRs) flanking the transgene. The transgene may comprise a therapeutic nucleic acid encoding a therapeutic gene expression product. Due to the limited packaging capacity of the rAAV (˜5 kB), in some cases, a longer transgene may be split between two AAV vectors, the first with 3′ splice donor and the second with a 5′ splice acceptor. Upon co-infection of a cell, concatemers form, which are spliced together to express a full-length transgene.

A transgene is generally inserted so that its expression is driven by the endogenous promoter at the integration site, namely the promoter that drives expression of the endogenous gene into which a transgene is inserted. In some instances, a transgene comprises a promoter and/or enhancer, for example a constitutive promoter or an inducible or tissue/cell specific promoter. As a non-limiting example, the promoter may be CMV promoter, a CMV-β-Actin-intron-β-Globin hybrid promoter (CAG), CBA promoter, FRDA or FXN promoter, UBC promoter, GUSB promoter, NSE promoter, Synapsin promoter, MeCP2 promoter, GFAP promoter, H1 promoter, U6 promoter, NFL promoter, NFH promoter, SCN8A promoter, or PGK promoter. As a non-limiting example, promoters can be tissue-specific expression elements include, but are not limited to, human elongation factor 1α-subunit (EF1α), immediate-early cytomegalovirus (CMV), chicken β-actin (CBA) and its derivative CAG, the β glucuronidase (GUSB), and ubiquitin C (UBC). The transgene may include a tissue-specific expression elements for neurons such as, but not limited to, neuron-specific enolase (NSE), platelet-derived growth factor (PDGF), platelet-derived growth factor B-chain (PDGF-β), the synapsin (Syn), the methyl-CpG binding protein 2 (MeCP2), Ca2+/calmodulin-dependent protein kinase II (CaMKII), metabotropic glutamate receptor 2 (mGluR2), NFL, NFH, np32, PPE, Enk and EAAT2 promoters. The transgene may comprise a tissue-specific expression element for astrocytes such as, but not limited to, the glial fibrillary acidic protein (GFAP) and EAAT2 promoters. The transgene may comprise tissue-specific expression elements for oligodendrocytes such as, but not limited to, the myelin basic protein (MBP) promoter.

In some embodiments, the promoter is less than 1 kb. The promoter may have a length of 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800 or more than 800. The promoter may have a length between 200-300, 200-400, 200-500, 200-600, 200-700, 200-800, 300-400, 300-500, 300-600, 300-700, 300-800, 400-500, 400-600, 400-700, 400-800, 500-600, 500-700, 500-800, 600-700, 600-800 or 700-800. The promoter may provide expression of the therapeutic gene expression product for a period of time in targeted tissues such as, but not limited to, the brain. Expression of the therapeutic gene expression product may be for a period of 1 hour, 2, hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 2 days, 3 days, 4 days, 5 days, 6 days, 1 week, 8 days, 9 days, 10 days, 11 days, 12 days, 13 days, 2 weeks, 15 days, 16 days, 17 days, 18 days, 19 days, 20 days, 3 weeks, 22 days, 23 days, 24 days, 25 days, 26 days, 27 days, 28 days, 29 days, 30 days, 31 days, 1 month, 2 months, 3 months, 4 months, 5 months, 6 months, 7 months, 8 months, 9 months, 10 months, 11 months, 1 year, 13 months, 14 months, 15 months, 16 months, 17 months, 18 months, 19 months, 20 months, 21 months, 22 months, 23 months, 2 years, 3 years, 4 years, 5 years, 6 years, 7 years, 8 years, 9 years, 10 years, 11 years, 12 years, 13 years, 14 years, 15 years, 16 years, 17 years, 18 years, 19 years, 20 years, 21 years, 22 years, 23 years, 24 years, 25 years, 26 years, 27 years, 28 years, 29 years, 30 years, 31 years, 32 years, 33 years, 34 years, 35 years, 36 years, 37 years, 38 years, 39 years, 40 years, 41 years, 42 years, 43 years, 44 years, 45 years, 46 years, 47 years, 48 years, 49 years, 50 years, 55 years, 60 years, 65 years, or more than 65 years. Expression of the payload may be for 1-5 hours, 1-12 hours, 1-2 days, 1-5 days, 1-2 weeks, 1-3 weeks, 1-4 weeks, 1-2 months, 1-4 months, 1-6 months, 2-6 months, 3-6 months, 3-9 months, 4-8 months, 6-12 months, 1-2 years, 1-5 years, 2-5 years, 3-6 years, 3-8 years, 4-8 years or 5-10 years or 10-15 years, or 15-20 years, or 20-25 years, or 25-30 years, or 30-35 years, or 35-40 years, or 40-45 years, or 45-50 years, or 50-55 years, or 55-60 years, or 60-65 years.

An AAV vector can comprise a genome of a helper virus. Helper virus proteins are required for the assembly of a recombinant AAV (rAAV), and packaging of a transgene containing a heterologous nucleic acid into the rAAV. The helper virus genes are adenovirus genes E4, E2a and VA, that when expressed in the cell, assist with AAV replication. In some embodiments, an AAV vector comprises E2. In some embodiments, an AAV vector comprises E4. In some embodiments, an AAV vector comprises VA. In some instances, the AAV vector comprises one of helper virus proteins, or any combination.

The target gene or gene expression product for use in a transgene can be selected from ATP1A2, CACNAIA, SETD5, SHANK3, NF2, DNMT1, TCF4, RAI1, PEX1, ARSA, EIF2B5, EIF2B1, EIF2B2, NPC1, ADAR, MFSD8, STXBP1, PRICKLE2, PRRT2, IDUA, STX1B, Sarcoglycan Alpha (SGCA), glutamic acid decarboxylase 65 (GAD65), glutamic acid decarboxylase 67 (GAD67), CLN2, Nerve Growth Factor (NGF), glial cell derived neurotrophic factor (GDNF), Survival Of Motor Neuron 1, STXBP1, Telomeric (SMN1), Factor X (FIX), Retinoid Isomerohydrolase (RPE65), sarco/endoplasmic reticulum Ca2+-ATPase (SERCA2a), Glucocerebrosidase (GCase), galactocerebrosidase (GALC), CDKL5, Frataxin (FXN), Huntingtin (HTT), methyl-CpG binding protein 2 (MECP2), a peroxisomal biogenesis factor (PEX), progranulin (GRN), an antitubulin agent, copper-zinc superoxide dismutase (SOD1), iduronate 2 sulfatase (hIDS), Glucosylceramidase Beta (GBA), fragile X mental retardation 1 (FMR1), NPC Intracellular Cholesterol Transporter 1 (NPC1), SCN1A, C9orf72, NPS3 and a NLRP3 inflammasome. In some embodiments, the peroxisomal biogenesis factor (PEX) is selected from PEX1, PEX2, PEX3, PEX4, PEX5, PEX6, PEX7, PEX10, PEX11β, PEX12, PEX13, PEX14, PEX16, PEX19, and PEX26.

An AAV vector can comprise a viral genome comprising a nucleic acid encoding the recombinant AAV (rAAV) capsid protein described herein. The viral genome can comprise a Replication (Rep) gene encoding a Rep protein, and Capsid (Cap) gene encoding an AAP protein in the first open reading frame (ORF1) or a Cap protein in the second open reading frame (ORF2). The Rep protein is selected from Rep78, Rep68, Rep52, and Rep40. In some instances, the Cap gene is modified encoding a modified AAV capsid protein described herein. A wild-type Cap gene encodes three proteins, VP1, VP2, and VP3. In some cases, VP1 is modified. In some cases, VP2 is modified. In some cases, VP3 is modified. In some cases, all three VP1-VP3 are modified. The AAV vector can comprise nucleic acids encoding wild-type Rep78, Rep68, Rep52, Rep40 and AAP proteins.

In some instances, the AAV9 VP1 gene provided in SEQ ID NO: 56 shown in Table 4, is modified to include any one of SEQ ID NOS: 57-73. The AAV vector described herein may be used to produce a variant AAV capsid by the methods described herein.

TABLE 4
VP1 Capsid Protein Nucleic Acid Sequences

| SEQ ID NO: | Identifier | Sequence |
|---|---|---|
| 650 | AAV9 | ATGGCTGCCGATGGTTATCTTCCAGATTGGCTCGAGGACAA |
| | >AY530579.1 | CCTTAGTGAAGGAATTCGCGAGTGGTGGGCTTTGAAACCTG |
| | Adeno- | GAGCCCCTCAACCCAAGGCAAATCAACAACATCAAGACAA |
| | associated | CGCTCGAGGTCTTGTGCTTCCGGGTTACAAATACCTTGGACC |
| | virus 9 | CGGCAACGGACTCGACAAGGGGGAGCCGGTCAACGCAGCA |
| | isolate | GACGCGGCGGCCCTCGAGCACGACAAGGCCTACGACCAGC |
| | hu. 14 capsid | AGCTCAAGGCCGGAGACAACCCGTACCTCAAGTACAACCAC |
| | protein VP1 | GCCGACGCCGAGTTCCAGGAGCGGCTCAAAGAAGATACGTC |
| | (cap) gene, | TTTTGGGGGCAACCTCGGGCGAGCAGTCTTCCAGGCCAAAA |
| | complete | AGAGGCTTCTTGAACCTCTTGGTCTGGTTGAGGAAGCGGCT |
| | cds | AAGACGGCTCCTGGAAAGAAGAGGCCTGTAGAGCAGTCTCC |
| | | TCAGGAACCGGACTCCTCCGCGGGTATTGGCAAATCGGGTG |
| | | CACAGCCCGCTAAAAAGAGACTCAATTTCGGTCAGACTGGC |
| | | GACACAGAGTCAGTCCCAGACCCTCAACCAATCGGAGAACC |
| | | TCCCGCAGCCCCCTCAGGTGTGGGATCTCTTACAATGGCTTC |
| | | AGGTGGTGGCGCACCAGTGGCAGACAATAACGAAGGTGCC |
| | | GATGGAGTGGGTAGTTCCTCGGGAAATTGGCATTGCGATTC |
| | | CCAATGGCTGGGGGACAGAGTCATCACCACCAGCACCCGAA |
| | | CCTGGGCCCTGCCCACCTACAACAATCACCTCTACAAGCAA |
| | | ATCTCCAACAGCACATCTGGAGGATCTTCAAATGACAACGC |
| | | CTACTTCGGCTACAGCACCCCCTGGGGGTATTTTGACTTCAA |
| | | CAGATTCCACTGCCACTTCTCACCACGTGACTGGCAGCGAC |
| | | TCATCAACAACAACTGGGGATTCCGGCCTAAGCGACTCAAC |
| | | TTCAAGCTCTTCAACATTCAGGTCAAAGAGGTTACGGACAA |
| | | CAATGGAGTCAAGACCATCGCCAATAACCTTACCAGCACGG |
| | | TCCAGGTCTTCACGGACTCAGACTATCAGCTCCCGTACGTGC |
| | | TCGGGTCGGCTCACGAGGGCTGCCTCCCGCCGTTCCCAGCG |
| | | GACGTTTTCATGATTCCTCAGTACGGGTATCTGACGCTTAAT |
| | | GATGGAAGCCAGGCCGTGGGTCGTTCGTCCTTTTACTGCCTG |
| | | GAATATTTCCCGTCGCAAATGCTAAGAACGGGTAACAACTT |
| | | CCAGTTCAGCTACGAGTTTGAGAACGTACCTTTCCATAGCA |
| | | GCTACGCTCACAGCCAAAGCCTGGACCGACTAATGAATCCA |
| | | CTCATCGACCAATACTTGTACTATCTCTCAAAGACTATTAAC |
| | | GGTTCTGGACAGAATCAACAAACGCTAAAATTCAGTGTGGC |
| | | CGGACCCAGCAACATGGCTGTCCAGGGAAGAAACTACATAC |
| | | CTGGACCCAGCTACCGACAACAACGTGTCTCAACCACTGTG |
| | | ACTCAAAACAACAACAGCGAATTTGCTTGGCCTGGAGCTTC |
| | | TTCTTGGGCTCTCAATGGACGTAATAGCTTGATGAATCCTGG |
| | | ACCTGCTATGGCCAGCCACAAAGAAGGAGAGGACCGTTTCT |
| | | TTCCTTTGTCTGGATCTTTAATTTTTGGCAAACAAGGAACTG |
| | | GAAGAGACAACGTGGATGCGGACAAAGTCATGATAACCAA |
| | | CGAAGAAGAAATTAAAACTACTAACCCGGTAGCAACGGAG |
| | | TCCTATGGACAAGTGGCCACAAACCACCAGAGTGCCCAAGC |
| | | ACAGGCGCAGACCGGCTGGGTTCAAAACCAAGGAATACTTC |
| | | CGGGTATGGTTTGGCAGGACAGAGATGTGTACCTGCAAGGA |
| | | CCCATTTGGGCCAAAATTCCTCACACGGACGGCAACTTTCA |
| | | CCCTTCTCCGCTGATGGGAGGGTTTGGAATGAAGCACCCGC |
| | | CTCCTCAGATCCTCATCAAAAACACACCTGTACCTGCGGAT |
| | | CCTCCAACGGCCTTCAACAAGGACAAGCTGAACTCTTTCAT |
| | | CACCCAGTATTCTACTGGCCAAGTCAGCGTGGAGATCGAGT |
| | | GGGAGCTGCAGAAGGAAAACAGCAAGCGCTGGAACCCGGA |
| | | GATCCAGTACACTTCCAACTATTACAAGTCTAATAATGTTGA |
| | | ATTTGCTGTTAATACTGAAGGTGTATATAGTGAACCCCGCCC |
| | | CATTGGCACCAGATACCTGACTCGTAATCTGTAA |

### Methods of Producing rAAVs

Disclosed herein are methods of producing the AAV capsids comprising the AAV capsid proteins and viral vector encoding a therapeutic nucleic acid. The AAV capsid proteins are produced by introducing into a cell (e.g., immortalized stem cell) a first vector containing a transgene cassette flanked by inverted terminal repeat (ITR) sequences from a parental AAV virus (the transgene cassette has a promoter sequence that drives transcription of a heterologous nucleic acid in the nucleus of the target cell), a second vector encoding the AAV genome with a AAV capsid protein (encoding the AAV Rep gene as well as the modified Cap gene for the variant being produced), and a third vector encoding helper virus proteins, required for assembly of the AAV capsid structure and packaging of the transgene in the modified AAV capsid structure. The assembled AAV capsid can be isolated and purified from the cell using suitable methods known in the art.

The transgenes contained in a recombinant AAV (rAAV) vector and encapsidated by the AAV capsid proteins of the present disclosure are also provided herein. The transgenes disclosed herein are delivered to a subject for a variety of purposes, such as to treat a disease or condition in the subject. The transgene can be gene editing components that modulate the activity or expression of a target gene or gene expression product. Alternatively, the transgene is a gene encoding a therapeutic gene expression product that is effective to modulate the activity or expression of itself, or another target gene or gene expression product.

Aspects disclosed herein provide methods of manufacturing rAAV virus or virus particles comprising: (a) introducing into a cell a nucleic acid comprising: (i) first vector containing a transgene cassette flanked by inverted terminal repeat (ITR) sequences from a parental AAV virus (the transgene cassette has a promoter sequence that drives transcription of a heterologous nucleic acid in the nucleus of the target cell); (ii) a second vector encoding the AAV genome with a AAV capsid protein of the present invention; and (iii) a vector encoding helper virus proteins, required for assembly of the AAV capsid structure and packaging of the transgene in the modified AAV capsid structure; (b) expressing in the cell the AAV capsid protein described herein; (c) assembling an AAV particle comprising the AAV capsid proteins disclosed herein; and (d) packaging the AAV particle. In some instances, the cell is mammalian. In some instances, the cell is immortalized. In some instances, the immortalized cell is an embryonic stem cell. In some instances, the embryonic stem cell is a human embryonic stem cell. In some instances, the human embryonic stem cell is a human embryonic kidney 293 (HEK-293) cell. In some instances, the Cap gene is derived from the deoxyribose nucleic acid (DNA) provided in SEQ ID NO: 6. In some instances, the 5′ ITR and the 3′ ITR are derived from an AAV2 serotype. In some instances, the 5′ ITR and the 3′ ITR are derived from an AAV5 serotype. In some instances, the 5′ ITR and the 3′ ITR are derived from an AAV9 serotype. In some instances, the first nucleic acid sequence and the second nucleic acid sequence are in trans. In some instances, the first nucleic acid sequence and the second nucleic acid sequence are in cis. In some instances, the first nucleic acid sequence, the second nucleic acid sequence and the third nucleic acid sequence, are in trans.

The Cap gene disclosed here comprises any one of SEQ ID NOS:57-73 from Table 4, which are DNA sequences encoding the modified AAV capsid protein portions of the present disclosure.

In some instances, the methods comprise packing the first nucleic acid sequence encoding the therapeutic gene expression product such that it becomes encapsidated by the modified AAV capsid protein. In some embodiments, the rAAV particles are isolated, concentrated, and purified using suitable viral purification methods, such as those described herein.

In some cases, rAAVs of the present disclosure are generated using the methods described in Challis, R. C. et al. Nat. Protoc. 14, 379 (2019). Briefly, triple transfection of HEK293T cells (ATCC) using polyethylenimine (PEI) is performed, viruses are collected after 120 hours from both cell lysates and media and purified over iodixanol. In a non-limiting example, the rAAVs are generated by triple transfection of precursor cells (e.g., HEK293T) cells using a standard transfection protocol (e.g., PEI). Viral particles are harvested from the media after a period of time (e.g., 72 h post transfection) and from the cells and media at a later point in time (e.g., 120 h post transfection). Virus present in the media is concentrated by precipitation with 8% polyethylene glycol (PEG) and 500 mM sodium chloride and the precipitated virus is added to the lysates prepared from the collected cells. The viruses are purified over iodixanol (Optiprep, Sigma) step gradients (15%, 25%, 40% and 60%). Viruses are concentrated and formulated in PBS. Virus titers are determined by measuring the number of DNaseI-resistant vector genome copies (VGs) using qPCR and the linearized genome plasmid as a control.

The cell can be selected from a human, a primate, a murine, a feline, a canine, a porcine, an ovine, a bovine, an equine, an epine, a caprine and a lupine host cell. In some instances, the cell is a progenitor or precursor cell, such as a stem cell. In some instances, the stem cell is a mesenchymal cell, embryonic stem cell, induced pluripotent stem cell (iPSC), fibroblast or other tissue specific stem cell. The cell can be immortalized. In some cases, the immortalized cell is a HEK293 cell. In some instances, the cell is a differentiated cell. Based on the disclosure provided, it is expected that this system can be used in conjunction with any transgenic line expressing a recombinase in the target cell type of interest to develop AAV capsids that more efficiently transduce that target cell population.

### Methods of Treatment

Disclosed herein are methods of treating a disease or condition, or a symptom of the disease or condition, in a subject, comprising administrating of therapeutically effective amount of one or more compositions (e.g., rAAV particle, AAV vector, pharmaceutical composition) disclosed herein to the subject. In some embodiments, the composition is a rAAV capsid protein described herein. In some embodiments, the composition is an isolated and purified rAAV capsid protein described herein. In some embodiments, the rAAV particle encapsidates an AAV vector comprising a transgene (e.g., therapeutic nucleic acid). In some embodiments, the composition is a rAAV capsid protein described herein conjugated with a therapeutic agent disclosed herein. In some embodiments, the composition is a pharmaceutical composition comprising the rAAV particle and a pharmaceutically acceptable carrier. In some embodiments, the one or more compositions are administered to the subject alone (e.g., stand-alone therapy). In some embodiments, the composition is a first-line therapy for the disease or condition. In some embodiments, the composition is a second-line, third-line, or fourth-line therapy, for the disease or condition.

Recombinant adeno-associated virus (rAAV) mediated gene delivery leverages the AAV mechanism of viral transduction for nuclear expression of an episomal heterologous nucleic acid (e.g., a transgene, therapeutic nucleic acid). Upon delivery to a host in vivo environment, a rAAV will (1) bind or attach to cellular surface receptors on the target cell, (2) endocytose, (3) traffic to the nucleus, (4) uncoat the virus to release the encapsidated heterologous nucleic acid, (5) convert of the heterologous nucleic acid from single-stranded to double-stranded DNA as a template for transcription in the nucleus, and (6) transcribe of the episomal heterologous nucleic acid in the nucleus of the host cell (“transduction”). rAAVs engineered to have an increased transduction enrichment (transcription of the episomal heterologous nucleic acid in the host cell) are desirable for gene therapy applications.

Aspects disclosed herein provide methods of treating a disease or condition in a subject, the method comprising administering to the subject a therapeutically effective amount of the rAAV of the present disclosure, or the pharmaceutical formulation of the present disclosure, wherein the gene product is a therapeutic gene product. In some embodiments, the administering is by intracranial, intraventricular, intracerebroventricular, intravenous, intraarterial, intranasal, intrathecal, intracisternae magna, or subcutaneous.

Provided here, are methods of treating a disease or a condition associated with an aberrant expression or activity of a target gene or gene expression product thereof, the method comprising modulating the expression or the activity of a target gene or gene expression product in a subject by administering a rAAV encapsidating a heterologous nucleic acid of the present disclosure. In some instances, the expression or the activity of the target gene or gene expression product is decreased, relative to that in a normal (non-diseased) individual; and administering the rAAV to the subject is sufficient to increase the expression of the activity of the target gene or gene expression product. In some instances, the expression or the activity of the gene or gene expression product is increased, relative to that in a normal individual; and administering the rAAV to the subject is sufficient to decrease the expression or the activity of the target gene or gene expression product. In a non-limiting example, a subject diagnosed with Alzheimer's disease, which is caused, in some cases, by a gain-of-function of a Presenilin 1 and/or Presenilin 2 (encoded by the gene PSEN1 and PSEN2, respectively) is administered a rAAV disclosed herein encapsidating a therapeutic nucleic acid that is a silencing RNA (siRNA), or other RNAi with a loss-of-function effect on PSEN1 mRNA.

Also provided are methods of preventing a disease or condition disclosed herein in a subject comprising administering to the subject a therapeutically effective amount of an rAAV vector comprising a nucleic acid sequence encoding a therapeutic gene expression product described herein. The rAAV vector may be encapsidated in the modified capsid protein or rAAV viral particle described herein. In some instances, the therapeutic gene expression product is effective to modulate the activity or expression of a target gene or gene expression product.

Disclosed herein are methods of treating a disease or condition in a subject by administering a composition comprising a rAAV disclosed herein. An advantage of the rAAVs disclosed herein, is that the rAAV may be used to treat virtually any disease or condition that would benefit from a transgene therapy, including but not limited to spinal muscular atrophy (SMA), amyotrophic lateral sclerosis (ALS), Parkinson's disease, Pompe disease, mucopolysaccharidosis type II, fragile X syndrome, STXBP1 encephalopathy, Krabbe disease, Huntington's disease, Alzheimer's disease, Battens disease, lysosomal storage disorders, glioblastoma multiforme, Rett syndrome, Leber's congenital amaurosis, Late infantile neuronal ceroid lipofuscinosis (LINCL), chronic pain, stroke, spinal cord injury, traumatic brain injury and lysosomal storage disorders.

In some cases, the disease or condition is localized to a particular in vivo environment in the subject, e.g., the brain. The compositions of the present disclosure are particularly useful for the treatment of the diseases or conditions described herein because they specifically or more efficiently target the in vivo environment and deliver a therapeutic nucleic acid engineered to modulate the activity or the expression of a target gene expression product involved with the pathogenesis or pathology of the disease or condition.

Provided herein are methods of treating a disease or a condition, or a symptom of the disease or condition, in a subject, comprising: (a) diagnosing a subject with a disease or a condition affecting a target in vivo environment; and (b) treating the disease or the condition by administering to the subject a therapeutically effective amount of a composition disclosed herein (e.g., rAAV particle, AAV vector, pharmaceutical composition), wherein the composition is engineered with an increased specificity for the target in vivo environment.

Disclosed herein are methods of treating a disease or a condition, or a symptom of the disease or the condition, afflicting a target in a subject comprising: (a) administering to the subject a composition (e.g., rAAV particle, AAV vector, pharmaceutical composition); and (b) expressing the therapeutic nucleic acid into a target in vivo environment in the subject with an increased transduction enrichment.

In some embodiments, methods further comprise reducing or ablating delivery of the heterologous nucleic acid in an off-target in vivo environment, such as the liver. In some embodiments, delivery is characterized by an increase in enrichment of transduction (e.g., of the heterologous nucleic acid) in the brain.

In some embodiments, methods of treating a disease or condition affecting the brain comprise administering a rAAV particle to a brain in a subject, the rAAV particle comprising an rAAV capsid protein comprising an insertion of about, five, six, or seven amino acids of an amino acid sequence provided in Tables 1-3, FIG. 1 and Formula I, at an amino acid position 588-589 in a parental AAV capsid protein. In some embodiments, methods of treating a disease or condition affecting the brain comprise administering a rAAV particle to a brain in a subject, the rAAV particle comprising an rAAV capsid protein comprising an insertion of about, five, six, or seven amino acids of an amino acid sequence as well as one or more substitution at amino acid found at amino acid positions 587-590 [AQAQ] such as provided in Tables 1-3, FIG. 1 and Formula I. In some embodiments, the parental AAV capsid protein is AAV9 capsid protein (for e.g., provided in SEQ ID NO: 1.

Also provided are methods of modulating a target gene expression product, the methods comprising administering to a subject in need thereof a composition (e.g., rAAV particle, AAV vector, pharmaceutical composition) disclosed herein. For example, methods provided herein comprise administering to a subject a rAAV with a rAAV capsid protein encapsidating a viral vector comprising a heterologous nucleic acid that modulates the expression or the activity of the target gene expression product.

The term “normal individual” refers to an individual that is not afflicted with the disease or the condition characterized by the variation in expression or activity of the gene or gene expression product thereof.

In some embodiments, the disease or condition of the brain selected from Absence of the Septum Pellucidum, Acid Lipase Disease, Acid Maltase Deficiency, Acquired Epileptiform Aphasia, Acute Disseminated Encephalomyelitis, Attention Deficit-Hyperactivity Disorder (ADHD), Adie's Pupil, Adie's Syndrome, Adrenoleukodystrophy, Agenesis of the Corpus Callosum, Agnosia, Aicardi Syndrome, Aicardi-Goutieres Syndrome Disorder, AIDS—Neurological Complications, Alexander Disease, Alpers' Disease, Alternating Hemiplegia, Alzheimer's Disease, Amyotrophic Lateral Sclerosis (ALS), Anencephaly, Aneurysm, Angelman Syndrome, Angiomatosis, Anoxia, Antiphospholipid Syndrome, Aphasia, Apraxia, Arachnoid Cysts, Arachnoiditis, Arnold-Chiari Malformation, Arteriovenous Malformation, Asperger Syndrome, Ataxia, Ataxia Telangiectasia, Ataxias and Cerebellar or Spinocerebellar Degeneration, Atrial Fibrillation and Stroke, Attention Deficit-Hyperactivity Disorder, Autism Spectrum Disorder, Autonomic Dysfunction, Back Pain, Barth Syndrome, Batten Disease, Becker's Myotonia, Behcet's Disease, Bell's Palsy, Benign Essential Blepharospasm, Benign Focal Amyotrophy, Benign Intracranial Hypertension, Bernhardt-Roth Syndrome, Binswanger's Disease, Blepharospasm, Bloch-Sulzberger Syndrome, Brachial Plexus Birth Injuries, Brachial Plexus Injuries, Bradbury-Eggleston Syndrome, Brain and Spinal Tumors, Brain Aneurysm, Brain Injury, Brown-Sequard Syndrome, Bulbospinal Muscular Atrophy, Cerebral Autosomal Dominant Arteriopathy with Subcortical Infarcts and Leukoencephalopathy (CADASIL), Canavan Disease, Carpal Tunnel Syndrome, Causalgia, Cavernomas, Cavernous Angioma, Cavernous Malformation, Central Cervical Cord Syndrome, Central Cord Syndrome, Central Pain Syndrome, Central Pontine Myelinolysis, Cephalic Disorders, Ceramidase Deficiency, Cerebellar Degeneration, Cerebellar Hypoplasia, Cerebral Aneurysms, Cerebral Arteriosclerosis, Cerebral Atrophy, Cerebral Beriberi, Cerebral Cavemous Malformation, Cerebral Gigantism, Cerebral Hypoxia, Cerebral Palsy, Cerebro-Oculo-Facio-Skeletal Syndrome (COFS), Charcot-Marie-Tooth Disease, Charcot-Marie-Tooth syndrome, classical rhizomelic chondrodysplasia punctata (RCDP), Chiari Malformation, Cholesterol Ester Storage Disease, Chorea, Choreoacanthocytosis, Chronic Inflammatory Demyelinating Polyneuropathy (CIDP), Chronic Orthostatic Intolerance, Chronic Pain, Cockayne Syndrome, Cockayne Syndrome Type II, Coffin Lowry Syndrome, Colpocephaly, Coma, Complex Regional Pain Syndrome, Congenital Facial Diplegia, Congenital Myasthenia, Congenital Myopathy, Congenital Vascular Cavernous Malformations, Corticobasal Degeneration, Cranial Arteritis, Craniosynostosis, Cree encephalitis, Creutzfeldt-Jakob Disease, Cumulative Trauma Disorders, Cushing's Syndrome, Cytomegalic Inclusion Body Disease, Cytomegalovirus Infection, Dancing Eyes-Dancing Feet Syndrome, Dandy-Walker Syndrome, Dawson Disease, Deafness, De Morsier's Syndrome, Dejerine-Klumpke Palsy, Dementia, Dementia—Multi-Infarct, Dementia—Semantic, Dementia—Subcortical, Dementia With Lewy Bodies, Dentate Cerebellar Ataxia, Dentatorubral Atrophy, Dermatomyositis, Developmental Dyspraxia, Devic's Syndrome, Diabetic Neuropathy, Diffuse Sclerosis, Dravet Syndrome, Duchenne muscular dystrophy, Dysautonomia, Dysgraphia, Dyslexia, Dysphagia, Dyspraxia, Dyssynergia Cerebellaris Myoclonica, Dyssynergia Cerebellaris Progressiva, Dystonias, Early Infantile Epileptic Encephalopathy, Empty Sella Syndrome, Encephalitis, Encephalitis Lethargica, Encephaloceles, Encephalopathy, Encephalopathy (familial infantile), Encephalotrigeminal Angiomatosis, Epilepsy, Epileptic Hemiplegia, Erb's Palsy, Erb—Duchenne and Dejerine-Klumpke Palsies, Essential Tremor, Extrapontine Myelinolysis, Fabry Disease, Fahr's Syndrome, Fainting, Familial Dysautonomia, Familial Hemangioma, Familial Idiopathic Basal Ganglia Calcification, Familial Periodic Paralyses, Familial Spastic Paralysis, Farber's Disease, Febrile Seizures, Fibromuscular Dysplasia, Fisher Syndrome, Floppy Infant Syndrome, Foot Drop, Fragile X syndrome, Friedreich's Ataxia, Frontotemporal Dementia (FTD), Gaucher Disease, Generalized Gangliosidoses, Gerstmann's Syndrome, Gerstmann-Straussler-Scheinker Disease, Giant Axonal Neuropathy, Giant Cell Arteritis, Giant Cell Inclusion Disease, glioblastoma, Globoid Cell Leukodystrophy, Glossopharyngeal Neuralgia, Glycogen Storage Disease, Guillain-Barre Syndrome, Hallervorden-Spatz Disease, Head Injury, Headache, Hemicrania Continua, Hemifacial Spasm, Hemiplegia Alterans, Hereditary Neuropathies, Hereditary Spastic Paraplegia, Heredopathia Atactica Polyneuritiformis, Herpes Zoster, Herpes Zoster Oticus, Hirayama Syndrome, Holmes-Adie syndrome, Holoprosencephaly, HTLV-1 Associated Myelopathy, Hughes Syndrome, Huntington's Disease, Hydranencephaly, Hydrocephalus, Hydrocephalus—Normal Pressure, Hydromyelia, Hypercortisolism, Hypersomnia, Hypertonia, Hypotonia, Hypoxia, Immune-Mediated Encephalomyelitis, Inclusion Body Myositis, Incontinentia Pigmenti, Infantile Hypotonia, Infantile Neuroaxonal Dystrophy, Infantile Phytanic Acid Storage Disease, Infantile Refsum Disease (IRD), Infantile Spasms, Inflammatory Myopathies, Iniencephaly, Intestinal Lipodystrophy, Intracranial Cysts, Intracranial Hypertension, Isaacs' Syndrome, Joubert Syndrome, Kearns-Sayre Syndrome, Kennedy's Disease, Kinsbourne syndrome, Kleine-Levin Syndrome, Klippel-Feil Syndrome, Klippel-Trenaunay Syndrome (KTS), Kliiver-Bucy Syndrome, Korsakoff s Amnesic Syndrome, Krabbe Disease, Kugelberg-Welander Disease, Kuru, Lambert-Eaton Myasthenic Syndrome, Landau-Kleffner Syndrome, Lateral Femoral Cutaneous Nerve Entrapment, Lateral Medullary Syndrome, Learning Disabilities, Leigh's Disease, Lennox-Gastaut Syndrome, Lesch-Nyhan Syndrome, Leukodystrophy, Levine-Critchley Syndrome, Lewy Body Dementia, Lipid Storage Diseases, Lipoid Proteinosis, Lissencephaly, Locked-In Syndrome, Lou Gehrig's Disease, Lupus—Neurological Sequelae, Lyme Disease—Neurological Complications, Machado-Joseph Disease, Macrencephaly, Maple syrup urine disease, Megalencephaly, Melkersson-Rosenthal Syndrome, Meningitis, Meningitis and Encephalitis, Menkes Disease, Menkes syndrome, Meralgia Paresthetica, Metachromatic Leukodystrophy, Microcephaly, Migraine, Miller Fisher Syndrome, Mini Stroke, Mitochondrial Myopathy, Moebius Syndrome, Monomelic Amyotrophy, Motor Neuron Diseases, Moyamoya Disease, Mucolipidoses, Mucopolysaccharidosis, Mucopolysaccharidosis II, Multi-Infarct Dementia, Multifocal Motor Neuropathy, Multiple Sclerosis, Multiple System Atrophy, Multiple System Atrophy with Orthostatic Hypotension, Muscular Dystrophy, Myasthenia—Congenital, Myasthenia Gravis, Myelinoclastic Diffuse Sclerosis, Myoclonic Encephalopathy of Infants, Myoclonus, Myopathy, Myopathy—Congenital, Myopathy—Thyrotoxic, Myotonia, Myotonia Congenita, Myotonic dystrophy, Narcolepsy, Neuroacanthocytosis, Neurodegeneration with Brain Iron Accumulation, Neurofibromatosis, Neuroleptic Malignant Syndrome, Neurological Complications of AIDS, Neurological Complications of Lyme Disease, Neurological Consequences of Cytomegalovirus Infection, Neurological Manifestations of Pompe Disease, Neurological Sequelae Of Lupus, Neuromyelitis Optica, Neuromyotonia, Neuronal Ceroid Lipofuscinosis, Neuronal Migration Disorders, Neuropathy—Hereditary, Neurosarcoidosis, Neurosyphilis, Neurotoxicity, Nevus Cavernosus, Niemann-Pick Disease, O'Sullivan-McLeod Syndrome, Occipital Neuralgia, Ohtahara Syndrome, Olivopontocerebellar Atrophy, Opsoclonus Myoclonus, Orthostatic Hypotension, Overuse Syndrome, Pain—Chronic, Pantothenate Kinase-Associated Neurodegeneration, Paraneoplastic Syndromes, Paresthesia, Parkinson's Disease, Paroxysmal Choreoathetosis, Paroxysmal Hemicrania, Parry-Romberg, Pelizaeus-Merzbacher Disease, Pena Shokeir II Syndrome, Perineural Cysts, Periodic Paralyses, Peripheral Neuropathy, Periventricular Leukomalacia, Persistent Vegetative State, Pervasive Developmental Disorders, Phenylketonuria, Phytanic Acid Storage Disease, Pick's Disease, Pinched Nerve, Piriformis Syndrome, Pituitary Tumors, Polymyositis, Pompe Disease, Porencephaly, Post-Polio Syndrome, Postherpetic Neuralgia, Postinfectious Encephalomyelitis, Postural Hypotension, Postural Orthostatic Tachycardia Syndrome, Postural Tachycardia Syndrome, Prader-Willi syndrome, Primary Dentatum Atrophy, Primary Lateral Sclerosis, Primary Progressive Aphasia, Prion Diseases, Progressive Hemifacial Atrophy, Progressive Locomotor Ataxia, Progressive Multifocal Leukoencephalopathy, Progressive Sclerosing Poliodystrophy, Progressive Supranuclear Palsy, Prosopagnosia, Pseudo-Torch syndrome, Pseudotoxoplasmosis syndrome, Pseudotumor Cerebri, Psychogenic Movement, Ramsay Hunt Syndrome I, Ramsay Hunt Syndrome II, Rasmussen's Encephalitis, Reflex Sympathetic Dystrophy Syndrome, Refsum Disease, Refsum Disease—Infantile, Repetitive Motion Disorders, Repetitive Stress Injuries, Restless Legs Syndrome, Retrovirus-Associated Myelopathy, Rett Syndrome, Reye's Syndrome, Rheumatic Encephalitis, Riley-Day Syndrome, Sacral Nerve Root Cysts, Saint Vitus Dance, Salivary Gland Disease, Sandhoff Disease, Schilder's Disease, Schizencephaly, Seitelberger Disease, Seizure Disorder, Semantic Dementia, Septo-Optic Dysplasia, Severe Myoclonic Epilepsy of Infancy (SMEI), Shaken Baby Syndrome, Shingles, Shy-Drager Syndrome, Sjogren's Syndrome, Sleep Apnea, Sleeping Sickness, Sotos Syndrome, Spasticity, Spina Bifida, Spinal Cord Infarction, Spinal Cord Injury, Spinal Cord Tumors, Spinal Muscular Atrophy, Spinocerebellar ataxia, Spinocerebellar Atrophy, Spinocerebellar Degeneration, Steele-Richardson-Olszewski Syndrome, Stiff-Person Syndrome, Striatonigral Degeneration, Stroke, Sturge-Weber Syndrome, STXBP1 encephalopathy, Subacute Sclerosing Panencephalitis, Subcortical Arteriosclerotic Encephalopathy, Short-lasting, Unilateral, Neuralgiform (SUNCT) Headache, Swallowing Disorders, Sydenham Chorea, Syncope, Syphilitic Spinal Sclerosis, Syringohydromyelia, Syringomyelia, Systemic Lupus Erythematosus, Tabes Dorsalis, Tangier disease, Tardive Dyskinesia, Tarlov Cysts, Tay-Sachs Disease, Temporal Arteritis, Tethered Spinal Cord Syndrome, Thomsen's Myotonia, Thoracic Outlet Syndrome, Thyrotoxic Myopathy, Tic Douloureux, Todd's Paralysis, Tourette Syndrome, Transient Ischemic Attack, Transmissible Spongiform Encephalopathies, Transverse Myelitis, Traumatic Brain Injury, Tremor, Trigeminal Neuralgia, Tropical Spastic Paraparesis, Troyer Syndrome, Tuberous Sclerosis, Vascular Erectile Tumor, Vasculitis Syndromes of the Central Nervous Systems, Von Economo's Disease, Von Hippel-Lindau Disease (VHL), Von Hippel-Lindau syndrome, Von Recklinghausen's Disease, Wallenberg's Syndrome, Werdnig-Hoffman Disease, Wernicke-Korsakoff Syndrome, West Syndrome, Whiplash, Whipple's Disease, Williams Syndrome, Wilson Disease, Wolman's Disease, X-Linked Spinal and Bulbar Muscular Atrophy and Zellweger syndrome.

In some embodiments, the pharmaceutical formulation comprises a therapeutic nucleic acid encoding a therapeutic gene expression product. In some instances, the therapeutic gene expression product is effective to modulate an activity or an expression of a target gene or gene expression product selected from ATP1A2, CACNAIA, SETD5, SHANK3, NF2, DNMT1, TCF4, RAI1, PEX1, ARSA, EIF2B5, EIF2B1, EIF2B2, NPC1, ADAR, MFSD8, STXBP1, PRICKLE2, PRRT2, IDUA, STX1B, Sarcoglycan Alpha (SGCA), glutamic acid decarboxylase 65 (GAD65), glutamic acid decarboxylase 67 (GAD67), CLN2, Nerve Growth Factor (NGF), glial cell derived neurotrophic factor (GDNF), Survival Of Motor Neuron 1, STXBP1, Telomeric (SMN1), Factor X (FIX), Retinoid Isomerohydrolase (RPE65), sarco/endoplasmic reticulum Ca2+-ATPase (SERCA2a), Glucocerebrosidase (GCase), galactocerebrosidase (GALC), CDKL5, Frataxin (FXN), Huntingtin (HTT), methyl-CpG binding protein 2 (MECP2), a peroxisomal biogenesis factor (PEX), progranulin (GRN), an antitubulin agent, copper-zinc superoxide dismutase (SOD1), iduronate 2 sulfatase (hIDS), Glucosylceramidase Beta (GBA), fragile X mental retardation 1 (FMR1), NPC Intracellular Cholesterol Transporter 1 (NPC1), SCN1A, C9orf72, NPS3 and a NLRP3 inflammasome. In some embodiments, the peroxisomal biogenesis factor (PEX) is selected from PEX1, PEX2, PEX3, PEX4, PEX5, PEX6, PEX7, PEX10, PEX11β, PEX12, PEX13, PEX14, PEX16, PEX19, and PEX26.

In some aspects, other examples of genes involved in neurologic or brain diseases or disorders include MAPT, IDUA, SNCA, ATXN2, Ube3a, GNS, HGSNAT, NAGLU, SGSH, CLN1, CLN3, CLN4, CLN5, CLN6, CLN7, CLN8, CTSD, ABCD1, HEXA, HEXB, ASM, ASPA, GLB1, AADC, MFN2, GNAO1, SYNGAP1, GRIN2A, GRIN2B, KCNQ2, EPM2A, NHLRC1, SLC6A1, SLC13A5, SURF1, GBE1, ATXN1, ATXN3, and ATXN7.

In some instances, the therapeutic gene expression product comprises gene editing components. In some instances, the gene editing components are selected from an artificial site-specific RNA endonuclease (ASRE), a zinc finger endonuclease (ZFN), a transcription factor like effector nuclease (TALEN), a clustered regularly interspaced short palindromic repeats (CRISPR)/Cas enzyme, and a CRISPR)/Cas guide RNA.

In some instances, the expression of a gene or expression or activity of a gene expression product is inhibited by the administration of the composition to the subject. In some instances, the expression of a gene or the expression or the activity of a gene expression product is enhanced by the administration of the composition to the subject.

### Formulations, Dosages, and Routes of Administration

Disclosed herein are methods comprising delivering a rAAV particle encapsidating a heterologous nucleic acid to the brain in a subject, the rAAV particle comprising (i) an increased transduction of the heterologous nucleic acid in the brain, wherein the rAAV particle has an rAAV capsid protein comprising an insertion of five, six, or seven amino acids of an amino acid sequence provided in Tables 1-3, FIG. 1 and Formula I, at an amino acid position 588-589 in a parental AAV capsid protein as well as one or more substitution at amino acid found at amino acid positions 587-590 [AQAQ] such as provided in Tables 1-3, FIG. 1 and Formula I. In various embodiments, the rAAV capsid protein may comprise one or more substitutions at amino acid positions 452-458 alone or in combination with the modifications above.

In general, methods disclosed herein comprise administering a therapeutic rAAV composition by systemic administration. In some instances, methods comprise administering a therapeutic rAAV composition by intravenous (“i.v.”) administration. One may administer therapeutic rAAV compositions by additional routes, such as subcutaneous injection, intramuscular injection, intradermal injection, transdermal injection, percutaneous administration, intranasal administration, intralymphatic injection, rectal administration intragastric administration, intraocular administration, intracerebroventricular administration, intrathecally, intracisternal, or any other suitable parenteral administration. Routes, dosage, time points, and duration of administrating therapeutics may be adjusted. In some embodiments, administration of therapeutics is prior to, or after, onset of either, or both, acute and chronic symptoms of the disease or condition. Other routes of delivery to the brain include, but are not limited to intracranial administration, lateral cerebroventricular administration, and endovascular administration.

An effective dose and dosage of pharmaceutical compositions to prevent or treat the disease or condition disclosed herein is defined by an observed beneficial response related to the disease or condition, or symptom of the disease or condition. Beneficial response comprises preventing, alleviating, arresting, or curing the disease or condition, or symptom of the disease or condition. In some embodiments, the beneficial response may be measured by detecting a measurable improvement in the presence, level, or activity, of biomarkers, transcriptomic risk profile, or intestinal microbiome in the subject. An “improvement,” as used herein refers to shift in the presence, level, or activity towards a presence, level, or activity, observed in normal individuals (e.g. individuals who do not suffer from the disease or condition). In instances wherein the therapeutic rAAV composition is not therapeutically effective or is not providing a sufficient alleviation of the disease or condition, or symptom of the disease or condition, then the dosage amount and/or route of administration may be changed, or an additional agent may be administered to the subject, along with the therapeutic rAAV composition. In some embodiments, as a patient is started on a regimen of a therapeutic rAAV composition, the patient is also weaned off (e.g., step-wise decrease in dose) a second treatment regimen.

In some cases, a dose of the pharmaceutical composition may comprise a concentration of infectious particles of at least or about 10⁷, 10⁸, 10⁹, 10¹⁰, 10¹¹, 10¹², 10¹³, 10¹⁴, 10¹⁵, 10¹⁶, or 10¹⁷. In some cases, the concentration of infectious particles is 2×10⁷, 2×10⁸, 2×10⁹, 2×10¹⁰, 2×10¹¹, 2×10¹², 2×10¹³, 2×10¹⁴, 2×10¹⁵, 2×10¹⁶, or 2×10¹⁷. In some cases, the concentration of the infectious particles is 3×10⁷, 3×10⁸, 3×10⁹, 3×10¹⁰, 3×10¹¹, 3×10¹², 3×10¹³, 3×10¹⁴, 3×10¹⁵, 3×10¹⁶, or 3×10¹⁷. In some cases, the concentration of the infectious particles is 4×10⁷, 4×10⁸, 4×10⁹, 4×10¹⁰, 4×10¹¹, 4×10¹², 4×10¹³, 4×10¹⁴, 4×10¹⁵, 4×10¹⁶, or 4×10¹⁷. In some cases, the concentration of the infectious particles is 5×10⁷, 5×10⁸, 5×10⁹, 5×10¹⁰, 5×10¹¹, 5×10¹², 5×10¹³, 5×10¹⁴, 5×10¹⁵, 5×10¹⁶, or 5×10¹⁷. In some cases, the concentration of the infectious particles is 6×10⁷, 6×10⁸, 6×10⁹, 6×10¹⁰, 6×10¹¹, 6×10¹², 6×10¹³, 6×10¹⁴, 6×10¹⁵, 6×10¹⁶, or 6×10¹⁷. In some cases, the concentration of the infectious particles is 7×10⁷, 7×10⁸, 7×10⁹, 7×10¹⁰, 7×10¹¹, 7×10¹², 7×10¹³, 7×10¹⁴, 7×10¹⁵, 7×10¹⁶, or 7×10¹⁷. In some cases, the concentration of the infectious particles is 8×10⁷, 8×10⁸, 8×10⁹, 8×10¹⁰, 8×10¹¹, 8×10¹², 8×10¹³, 8×10¹⁴, 8×10¹⁵, 8×10¹⁶, or 8×10¹⁷. In some cases, the concentration of the infectious particles is 9×10⁷, 9×10⁸, 9×10⁹, 9×10¹⁰, 9×10¹¹, 9×10¹², 9×10¹³, 9×10¹⁴, 9×10¹⁵, 9×10¹⁶, or 9×10¹⁷.

Disclosed herein, in some embodiments are formulations of pharmaceutically-acceptable excipients and carrier solutions suitable for delivery of the rAAV compositions described herein, as well as suitable dosing and treatment regimens for using the particular compositions described herein in a variety of treatment regimens. In some embodiments, the amount of therapeutic gene expression product in each therapeutically-useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In some embodiments, the pharmaceutical forms of the rAAV-based viral compositions suitable for injectable use include sterile aqueous solutions or dispersions and sterile powders for the extemporaneous preparation of sterile injectable solutions or dispersions. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride.

In some cases, for administration of an injectable aqueous solution, the solution may be suitably buffered, if necessary, and the liquid diluent first rendered isotonic with sufficient saline or glucose. These particular aqueous solutions are especially suitable for intravenous, intramuscular, subcutaneous and intraperitoneal administration. Some variation in dosage will necessarily occur depending on the condition of the subject being treated. The person responsible for administration will, in any event, determine the appropriate dose for the individual subject. Moreover, for human administration, preparations should meet sterility, pyrogenicity, and the general safety and purity standards as required by FDA Office of Biologics standards.

Disclosed herein are sterile injectable solutions comprising the rAAV compositions disclosed herein, which are prepared by incorporating the rAAV compositions disclosed herein in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. Injectable solutions may be advantageous for systemic administration, for example by intravenous or intrathecal administration.

Suitable dose and dosage administrated to a subject is determined by factors including, but not limited to, the particular therapeutic rAAV composition, disease condition and its severity, the identity (e.g., weight, sex, age) of the subject in need of treatment, and can be determined according to the particular circumstances surrounding the case, including, e.g., the specific agent being administered, the route of administration, the condition being treated, and the subject or host being treated.

The amount of rAAV compositions and time of administration of such compositions will be within the purview of the skilled artisan having benefit of the present teachings. It is likely, however, that the administration of therapeutically-effective amounts of the disclosed compositions may be achieved by a single administration, example, a single injection of sufficient numbers of infectious particles to provide therapeutic benefit to the patient undergoing such treatment. This is made possible, at least in part, by the fact that certain target cells (e.g., neurons) do not divide, obviating the need for multiple or chronic dosing.

In certain embodiments, the data obtained from cell culture assays and animal studies are used in formulating the therapeutically effective daily dosage range and/or the therapeutically effective unit dosage amount for use in mammals, including humans. In certain embodiments, the dosage range and/or the unit dosage amount varies within this range depending upon the dosage form employed and the route of administration utilized.

### Combination Therapies

A therapeutic rAAV may be used alone or in combination with an additional therapeutic agent (together, “therapeutic agents”). In some cases, a therapeutic rAAV as used herein is administered alone. The therapeutic agent may be administered together or sequentially in a combination therapy. The combination therapy may be administered within the same day, or may be administered one or more days, weeks, months, or years apart.

The additional therapeutic agent can comprise a small molecule. The additional therapeutic agent can comprise an antibody, or antigen-binding fragment. The additional therapeutic agent can include lipid nanoparticle-based therapies, anti-sense oligonucleotide therapies, as well as other viral therapies.

The additional therapeutic agent can comprise a cell-based therapy. Exemplary cell-based therapies include without limitation immune effector cell therapy, chimeric antigen receptor T-cell (CAR-T) therapy, natural killer cell therapy and chimeric antigen receptor natural killer (NK) cell therapy. Either NK cells, or CAR-NK cells, or a combination of both NK cells and CAR-NK cells can be used in combination with the methods disclosed herein. In some embodiments, the NK cells and CAR-NK cells are derived from human induced pluripotent stem cells (iPSC), umbilical cord blood, or a cell line. The NK cells and CAR-NK cells can comprise a cytokine receptor and a suicide gene. The cell-based therapy can comprise a stem cell therapy. The stem cell therapy may be embryonic or somatic stem cells. The stem cells may be isolated from a donor (allogeneic) or isolated from the subject (autologous). The stem cells may be expanded adipose-derived stem cells (eASCs), hematopoietic stem cells (HSCs), mesenchymal stem (stromal) cells (MSCs), or induced pluripotent stem cells (iPSCs) derived from the cells of the subject.

### Kits

Disclosed herein are kits comprising compositions disclosed herein. Also disclosed herein are kits for the treatment or prevention of a disease or conditions of the brain. In some instances, the disease or condition is cancer, a pathogen infection, pulmonary disease or condition, neurological disease, muscular disease, or an immune disorder, such as those described herein.

In one embodiment, a kit can include a therapeutic or prophylactic composition containing an effective amount of a composition of a rAAV particle encapsidating a recombinant AAV vector encoding a therapeutic nucleic acid (e.g., therapeutic nucleic acid) and a recombinant AAV (rAAV) capsid protein of the present disclosure. In another embodiment, a kit can include a therapeutic or prophylactic composition containing an effective amount of cells modified by the rAAV described herein (“modified cell”), in unit dosage form that express therapeutic nucleic acid. In some embodiments, a kit comprises a sterile container which can contain a therapeutic composition; such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments.

In some instances, the kit further comprises a cell. In some instances, the cell is mammalian. In some instances, the cell is immortalized. In some instances, the immortalized cell is an embryonic stem cell. In some instances, the embryonic stem cell is a human embryonic stem cell. In some instances, the human embryonic stem cell is a human embryonic kidney 293 (HEK-293) cell. In some instances, the kit further comprises an AAV vector comprising a heterologous nucleic acid encoding a therapeutic gene expression product. In some instances, the AAV vector is an episome.

In some cases, rAAV are provided together with instructions for administering the rAAV to a subject having or at risk of developing the disease or condition (e.g., disease of the brain). Instructions can generally include information about the use of the composition for the treatment or prevention of the disease or condition.

In some cases, the instructions include at least one of the following: description of the therapeutic rAAV composition; dosage schedule and administration for treatment or prevention of the disease or condition disclosed herein; precautions; warnings; indications; counter-indications; overdosage information; adverse reactions; animal pharmacology; clinical studies; and/or references. The instructions can be printed directly on the container (when present), or as a label applied to the container, or as a separate sheet, pamphlet, card, or folder supplied in or with the container. In some cases, instructions provide procedures for administering the rAAV to the subject alone. In some instances, the instructions provide that the rAAV is formulated for systemic delivery.

### Definitions

The terminology used herein is for the purpose of describing particular cases only and is not intended to be limiting. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

The term “about” or “approximately” means within an acceptable error range for the particular value as determined by one of ordinary skill in the art, which will depend in part on how the value is measured or determined, e.g., the limitations of the measurement system. For example, “about” can mean within 1 or more than 1 standard deviation, per the practice in the given value. Where particular values are described in the application and claims, unless otherwise stated the term “about” should be assumed to mean an acceptable error range for the particular value.

As used herein “consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) of the claimed disclosure, such as compositions for treating skin disorders like acne, eczema, psoriasis, and rosacea.

The terms “homologous,” “homology,” or “percent homology” are used herein to generally mean an amino acid sequence or a nucleic acid sequence having the same, or similar sequence to a reference sequence. Percent homology of sequences can be determined using the most recent version of BLAST, as of the filing date of this application.

The terms “increased,” or “increase” are used herein to generally mean an increase by a statically significant amount. In some embodiments, the terms “increased,” or “increase,” mean an increase of at least 10% as compared to a reference level, for example an increase of at least about 10%, at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, standard, or control. Other examples of “increase” include an increase of at least 2-fold, at least 5-fold, at least 10-fold, at least 20-fold, at least 50-fold, at least 100-fold, at least 1000-fold or more as compared to a reference level.

The terms, “decreased” or “decrease” are used herein generally to mean a decrease by a statistically significant amount. In some embodiments, “decreased” or “decrease” means a reduction by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g., absent level or non-detectable level as compared to a reference level), or any decrease between 10-100% as compared to a reference level. In the context of a marker or symptom, by these terms is meant a statistically significant decrease in such level. The decrease can be, for example, at least 10%, at least 20%, at least 30%, at least 40% or more, and is preferably down to a level accepted as within the range of normal for an individual without a given disease.

The terms “subject” is any organism. In some instances, the organism is a mammal. Non-limiting examples of mammal include, any member of the mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In certain aspects, the mammal is a human. The term “animal” as used herein comprises human beings and non-human animals. In one embodiment, a “non-human animal” is a mammal, for example a rodent such as rat or a mouse. In one embodiment, a “non-human primate” is a mammal, for example a monkey. In some instances, the subject is a patient, which as used herein, may refer to a subject diagnosed with a particular disease or disorder.

The term “gene,” as used herein, refers to a segment of nucleic acid that encodes an individual protein or RNA (also referred to as a “coding sequence” or “coding region”), optionally together with associated regulatory region such as promoter, operator, terminator and the like, which may be located upstream or downstream of the coding sequence.

The term “adeno-associated virus,” or “AAV” as used herein refers to the adeno-associated virus or derivatives thereof. Non-limited examples of AAV's include AAV type 1 (AAV1), AAV type 2 (AAV2), AAV type 3 (AAV3), AAV type 4 (AAV4), AAV type 5 (AAV5), AAV type 6 (AAV6), AAV type 7 (AAV7), AAV type 8 (AAV8), AAV type 9 (AAV9), AAV type 10 (AAV10), AAV type 11 (AAV11), AAV type 12 (AAV12), avian AAV, bovine AAV, canine AAV, equine AAV, primate AAV, non-primate AAV, and ovine AAV. In some instances, the AAV is described as a “Primate AAV,” which refers to AAV that infect primates. Likewise an AAV may infect bovine animals (e.g., “bovine AAV”, and the like). In some instances, the AAV is wildtype, or naturally occurring. In some instances, the AAV is recombinant.

The term “AAV capsid” as used herein refers to a capsid protein or peptide of an adeno-associated virus. In some instances, the AAV capsid protein is configured to encapsidate genetic information (e.g., a transgene, therapeutic nucleic acid, viral genome). In some instances, the AAV capsid of the instant disclosure is a modified AAV capsid, relative to a corresponding parental AAV capsid protein.

The term “tropism” as used herein refers to a quality or characteristic of the AAV capsid that may include specificity for, and/or an increase or a decrease in enrichment of, expressing the encapsidated genetic information into an in vivo environment, relative to a second in vivo environment. An in vivo environment, in some instances, is a cell-type. An in vivo environment, in some instances, is an organ or organ system.

The term “AAV vector” as used herein refers to nucleic acid polymer encoding genetic information related to the virus. The AAV vector may be a recombinant AAV vector (rAAV), which refers to an AAV vector generated using recombinatorial genetics methods. In some instances, the rAAV vector comprises at least one heterologous polynucleotide (e.g. a polynucleotide other than a wild-type or naturally occurring AAV genome such as a transgene).

The term “AAV particle” as used herein refers to an AAV virus, virion, AAV capsid protein or component thereof. In some cases, the AAV particle is modified relative to a parental AAV particle.

The term “gene product” of “gene expression product” refers to an expression product of a polynucleotide sequence such as, for e.g., a polypeptide, peptide, protein or RNA, including interfering RNA (e.g., siRNA, miRNA, shRNA) and messenger RNA (mRNA).

The term “heterologous” as used herein refers to a genetic element (e.g., coding region) or gene expression product (e.g., RNA, protein) that is derived from a genotypically distinct entity from that of the rest of the entity to which it is being compared.

The term “endogenous” as used herein refers to a genetic element (e.g., coding region) or gene expression product (e.g., RNA, protein) that is naturally occurring in or associated with an organism or a particular cell within the organism.

The terms “treat,” “treating,” and “treatment” as used herein refers to alleviating or abrogating a disorder, disease, or condition; or one or more of the symptoms associated with the disorder, disease, or condition; or alleviating or eradicating a cause of the disorder, disease, or condition itself. Desirable effects of treatment can include, but are not limited to, preventing occurrence or recurrence of disease, alleviation of symptoms, diminishing any direct or indirect pathological consequences of the disease, preventing metastasis, decreasing the rate of disease progression, amelioration or palliation of the disease state and remission or improved prognosis.

The term “therapeutically effective amount” refers to the amount of a compound or therapy that, when administered, is sufficient to prevent development of, or alleviate to some extent, one or more of the symptoms of a disorder, disease, or condition of the disease; or the amount of a compound that is sufficient to elicit biological or medical response of a cell, tissue, system, animal, or human that is being sought by a researcher, veterinarian, medical doctor, or clinician.

The term “pharmaceutically acceptable carrier,” “pharmaceutically acceptable excipient,” “physiologically acceptable carrier,” or “physiologically acceptable excipient” refers to a pharmaceutically acceptable material, composition, or vehicle, such as a liquid or solid filler, diluent, excipient, solvent, or encapsulating material. A component can be “pharmaceutically acceptable” in the sense of being compatible with the other ingredients of a pharmaceutical formulation. It can also be suitable for use in contact with the tissue or organ of humans and animals without excessive toxicity, irritation, allergic response, immunogenicity, or other problems or complications, commensurate with a reasonable benefit/risk ratio. See, Remington: The Science and Practice of Pharmacy, 21st Edition; Lippincott Williams & Wilkins: Philadelphia, PA, 2005; Handbook of Pharmaceutical Excipients, 5th Edition; Rowe et al., Eds., The Pharmaceutical Press and the American Pharmaceutical Association: 2005; and Handbook of Pharmaceutical Additives, 3rd Edition; Ash and Ash Eds., Gower Publishing Company: 2007; Pharmaceutical Preformulation and Formulation, Gibson Ed., CRC Press LLC: Boca Raton, FL, 2004).

The term “pharmaceutical composition” refers to a mixture of a compound disclosed herein with other chemical components, such as diluents or carriers. The pharmaceutical composition can facilitate administration of the compound to an organism. Multiple techniques of administering a compound exist in the art including, but not limited to, systemic administration.

Non-limiting examples of “sample” include any material from which nucleic acids and/or proteins can be obtained. As non-limiting examples, this includes whole blood, peripheral blood, plasma, serum, saliva, mucus, urine, semen, lymph, fecal extract, cheek swab, cells or other bodily fluid or tissue, including but not limited to tissue obtained through surgical biopsy or surgical resection. Alternatively, a sample can be obtained through primary patient derived cell lines, or archived patient samples in the form of preserved samples, or fresh frozen samples.

The term “in vivo” is used to describe an event that takes place in a subject's body.

The term “in vitro” is used to describe an event that takes places contained in a container for holding laboratory reagent such that it is separated from the biological source from which the material is obtained. In vitro assays can encompass cell-based assays in which living or dead cells are employed. In vitro assays can also encompass a cell-free assay in which no intact cells are employed.

The term “brain” means a tissue selected from brain, thalamus, cortex, putamen, lateral ventricles, medulla, the pons, the amygdala, the motor cortex, caudate, hypothalamus, striatum, ventral midbrain, neocortex, basal ganglia, hippocampus, cerebrum, cerebellum, brain stem, and spinal cord. The brain includes a variety of cortical and subcortical areas, including the frontal, temporal, occipital and parietal lobes.

The term “systemic delivery” is defined as a route of administration of medication or other substance into a circulatory system so that the entire body is affected. Administration can take place via enteral administration (absorption of the drug through the gastrointestinal tract) or parenteral administration (generally injection, infusion, or implantation). “Circulatory system” includes both blood or cerebrospinal fluid circulatory systems Examples of systemic administration for the brain include intraarterial, intravenous or intrathecal injection. Other examples include administration to the cerebrospinal fluid at any location, in the spine (i.e. but not limited to lumbar) or brain (i.e. but not limited to cisterna magna). The terms “systemic administration” and “systemic delivery” are used interchangeably.

The section headings used herein are for organizational purposes only and are not to be construed as limiting the subject matter described.

### EXAMPLES

### Example 1

### Method of Identifying the Modified Capsid Proteins in Cynomolgus Macaques

Of primary concern for the therapeutic applicability of engineered adeno-associated viruses (AAVs) is how well their transduction profiles translate to human application. While previous engineering efforts have focused on in vitro or in vivo rodent screening platforms due to the ease and flexibility of their use, screening efforts directly in non-human primates (NHPs) are much more likely to identify viruses that translate. Cynomolgus macaques, an old world NHP, were chosen for the engineering efforts. The efforts were focused on a region of the AAV9 capsid surface located at amino acid position 588, one of the most exposed loops on the capsid surface that is a variable region between natural AAV serotypes and has a role in receptor binding. Insertion of peptides between positions 588 and 589 has been studied in the past, and has resulted in novel receptor binding (AAV-PHP.B/AAV-PHP.eB binding of Ly6a on rodent brain endothelium to facilitate blood-brain barrier crossing and high transduction of the brain) and drastically altered capsid tropism. A library of viral capsids was created by performing a random 7 amino acid insertion at this site within AAV9, hoping for novel tropism toward the NHP brain. Through two rounds of screening via this process an rAAV was identified with a particular 7 amino acid peptide insertion conferring high brain tropism, and this rAAV was used as a parent capsid on which to make mutational substitutions in Round 3 screening.

Plasmids. The first-round viral DNA library was generated by amplification of a section of the AAV9 capsid genome between amino acids 450-599 using NNK degenerate primers (Integrated DNA Technologies, Inc., IDT) to insert seven random amino acids between amino acids 588 and 589 with all possible variations. The resulting library inserts were then introduced into the rAAV-ACap-in-rev-RNA plasmid via Gibson assembly as previously described. The resulting capsid DNA library, rAAV-Cap-Cag-GFP11, contained a diversity of ˜1.28 billion variants at the amino acid level. The second round viral DNA library was generated similarly to the first round, but instead of NNK degenerate primers inserted at the 588, a synthesized oligo pool (Twist Biosicence) was used to generate only selected variants in a UBC-Cap-DNA and CAG-Cap-DNA construct with CAP. The third round viral DNA library was generated similarly to the second, but a synthesized oligo pool of selected variants was ordered from IDT and used to generate capsids with amino acid substitutions in the 588 loop or 452 loop. This library contained a diversity of 10,000 variants at the amino acid level with 2 barcoded replicates for each variant.

The AAV2/9 REP-AAP-ACAP plasmid transfected into HEK293T cells to provide the Rep gene for library viral production prevents production of a wild-type AAV9 capsid during viral library production after a plausible recombination event between this plasmid co-transfected with the library plasmids at each stage containing the library inserts.

Viral production. Recombinant AAVs were generated according to established protocols. Briefly, immortalized HEK293T cells (ATCC) were quadruple transfected with four vectors using polyethylenimine (PEI). The first vector was the rAAV-Cap-in-cis-Lox library flanked by inverted terminal repeat (ITR) sequences from a parental AAV virus. The second vector was the AAV2/9 REP-AAP-ACAP plasmid. The third vector contains nucleic acids encoding helper virus proteins needed for viral assembly and packaging of the heterologous nucleic acid into the modified capsid structure. The fourth is a pUC-18 plasmid included to achieve the right PEI/DNA ratio for optimal transfection enrichment. Only 10 ng of rAAV-Cap-in-cis-Lox library DNA was transfected (per 150 mm plate) to decrease the likelihood of multiple library DNAs entering the same cell. Viral particles are harvested from the cells and media after 60 h post transfection. Virus present in the media is concentrated by precipitation with 8% polyethylene glycol and 500 mM sodium chloride and the precipitated virus is added to the lysates prepared from the collected cells. The viruses are purified over iodixanol (Optiprep, Sigma) step gradients (15%, 25%, 40%, and 60%). Viruses are concentrated and formulated in PBS. Virus titers are determined by measuring the number of DNaseI-resistant vector genome copies (VGs) using qPCR and the linearized genome plasmid as a control.

Animals. Cynomolgus macaque procedures were approved by the IACUC committee of Envol Biomedical. Cynomolgus macaques were born and raised in Envol Biomedical colonies and housed in family groups under standard conditions. They were fed ad libitum and received enrichment as part of the primate enrichment program for NHPs at Envol Biomedical. For AAV infusions, animals were screened for endogenous neutralizing antibodies (Nab). None of the animals that were screened showed any detectible blocking reaction at 1:10 dilution of serum. They were then group housed for several days and acclimated to a new room before injections. Animals were restrained and the test article was administered via intravenous infusion for 10 minutes. Two infant monkeys were used in each of the round 1 and round 2 screenings, and three infant monkeys in the round 3 screening. Activity and behavior were monitored daily over the course of in-life.

DNA/RNA recovery and sequencing. Viral libraries were injected into cynomolgus macaques at a dose of 1-3×10¹³ vg/kg animal and rAAV genomes were recovered two weeks post injection. Animals were euthanized and brain, spinal cord and liver were recovered, snap frozen, and placed into long-term storage at −80° C. as well as other peripheral tissues such as heart, spleen, adrenal, kidney, and quad. The brain was separated into 11-13 brain regions, and 100 mg of each brain region was homogenized in buffer using the MagMAX DNA ULTRA (A25597) and a Bead Ruptor 96 (OMNI, INC) and viral DNA was isolated according to the manufacturers recommended protocol. Recovered viral DNA was treated with RNase, and purified with a Zymo DNA Clean and Concentrator kit (D4033). Viral genomes were enriched by 25 cycles of PCR amplification with primers flanking the AA 452-588 region in the capsid genome using 50% of the total extracted viral DNA as a template. After Zymo DNA purification, samples were diluted 1:10 to 1:1000 depending on tissue type and each dilution further amplified around the library variable region with 10 cycles of PCR. Subsequently, samples were further amplified using custom primers with Illumina Indices for 10 more cycles. The amplification products were run on a 2% low-melting point agarose gel (ThermoFisher Scientific, 16520050) for better separation and recovery of the 600 bp band.

For the second and third round libraried only, packaged viral library DNA was isolated from the injected viral library by digestion of the viral capsid and purification of the contained ssDNA. These viral genomes were amplified by two PCR amplification steps, like the viral DNA extracted from tissue, to add adapters and indices for Illumina next-generation sequencing, and purified after gel electrophoresis. This viral library DNA, along with the viral DNA extracted from tissue, was sent for deep sequencing using an Illumina NextSeq 2000 system.

NGS data alignment and processing. Raw fastq files from NGS runs were processed with custom-built scripts (Capsida CapSeq Tools). For the first round library, the pipeline to process these datasets involved filtering to remove low-quality reads, utilizing a quality score for each sequence, and eliminating bias from PCR-induced mutations or high GC-content. The filtered dataset was then aligned by a perfect string match algorithm and trimmed to improve the alignment quality. Read counts for each sequence were pulled out and displayed by tissue, at which point all sequences found in the brain were compiled for formation of the second round library.

For the second and third round libraries read counts by tissue were similarly tabulated. Then, a read count of 1 was added to each sequence to remove 0 values, all brain regions for each sequence were summed together, and the read sequences for each codon replicate of a given 7-mer amino acid sequence were summed together to give a single value for each peptide insertion. Finally, the read count data was normalized to counts per million (Cpm). Enrichment values for each capsid variant were calculated via normalized cpm from [tissue of interest]/cpm from viral library.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

All publications, patents, and patent applications mentioned in this specification are herein incorporated by reference to the same extent as if each individual publication, patent, or patent application was specifically and individually indicated to be incorporated by reference.

What is claimed is:

1. An AAV capsid protein comprising a sequence provided in Table 1 or selected from the group consisting of SEQ ID NOs: 3-398.

2. The AAV capsid protein of claim 1, comprising an insertion sequence of Formula I
(I)
(SEQ ID NO: 2)
X¹-N-T-T-K-X²-X³
wherein X¹ is an amino acid selected from L, G, S, A, Q, and D; X² is an amino acid selected from S, P, Q, and N; and X³ is an amino acid selected from V, A, S, I, and F.

3. The AAV capsid protein of claim 2, wherein X¹ is L.

4. The AAV capsid protein of claim 2, wherein X¹ is G.

5. The AAV capsid protein of claim 2, wherein X² is S.

6. The AAV capsid protein of claim 2, wherein X³ is V.

7. The AAV capsid protein of claim 1, wherein the sequence is selected from the group consisting of SNTTKSV (SEQ ID NO: 631), QNTTKSV (SEQ ID NO: 632), ADLNTTKPVMQ (SEQ ID NO: 5), AQANTTKSVDQ (SEQ ID NO: 6), AQNNTTKSVDQ (SEQ ID NO: 7), AQLNTTKNVDQ (SEQ ID NO: 8), ADLNTTKSVAQ (SEQ ID NO: 9), AQDNTTKSVFQ (SEQ ID NO: 10), ADLNTTKSVSQ (SEQ ID NO: 11), AQGNTTKSVDQ (SEQ ID NO: 12), AQGNTTKSVQQ (SEQ ID NO: 13), and AQLNTTKSVDQ. (SEQ ID NO: 14).

8. The AAV capsid protein of claim 1, comprising AAV9 as a parent AAV.

9. The AAV capsid protein of claim 8, wherein the parent AAV comprises SEQ ID NO: 1.

10. The AAV capsid protein of claim 9, comprising a 7-mer insertion inserted into the parent AAV between amino acid 588 and amino acid 589 of the parent AAV, wherein positions 587-597 of the AAV capsid protein are selected from the sequence provided in Table 1 or selected from the group consisting of SEQ ID NOs: 3-398.

11. The AAV capsid protein of claim 1, wherein 60 copies of the AAV capsid protein are assembled into the AAV capsid.

12. The AAV capsid protein of claim 1, wherein the AAV capsid protein is present in VP1, VP2, and VP3 of the AAV capsid.

13. The AAV capsid protein of claim 1, further characterized by an increased transduction enrichment relative to AAV9 when measured in brain tissue in a subject when delivered to the subject systemically.

14. The AAV capsid protein of claim 1, further characterized by a decreased transduction enrichment relative to AAV9 when measured in liver tissue in a subject when delivered to the subject systemically.

15. The AAV capsid protein of claim 12, wherein the AAV capsid protein further comprises an amino acid substitution relative to the parent AAV comprising one or more of A587P, Q588D, A589M, A589D, or Q590G.

16. An AAV capsid protein comprising a sequence provided in Table 2 or selected from the group consisting of SEQ ID NOs: 400-630.

17. The AAV capsid protein of claim 16, comprising AAV9 as a parent AAV.

18. The AAV capsid protein of claim 17, wherein the parent AAV comprises SEQ ID NO: 1.

19. The AAV capsid protein of claim 18, comprising one or more substitutions at positions 452-458 relative to the parent AAV, wherein positions 452-458 of the AAV capsid protein are selected from the sequence provided in Table 2 or selected from the group consisting of SEQ ID NOs: 400-630.

20. The AAV capsid protein of claim 16, wherein 60 copies of the AAV capsid protein are assembled into the AAV capsid.

21. The AAV capsid protein of claim 16, wherein the AAV capsid protein is present in VPT, VP2, and VP3 of the AAV capsid.

22. The AAV capsid protein of claim 16, further characterized by an increased transduction enrichment relative to AAV9 when measured in brain tissue in a subject when delivered to the subject systemically.

23. The AAV capsid protein of claim 16, further characterized by a decreased transduction enrichment relative to AAV9 when measured in liver tissue in a subject when delivered to the subject systemically.

24. An AAV capsid protein comprising:
a first sequence provided in Table 1 or selected from the group consisting of SEQ ID NOs: 3-398; and
a second sequence provided in Table 2 or selected from the group consisting of SEQ ID NOs: 399-630.

25. The AAV capsid protein of claim 24, wherein the first sequence comprises an insertion sequence of Formula I
(I)
(SEQ ID NO: 2)
X¹-N-T-T-K-X²-X³
wherein X¹ is an amino acid selected from L, G, S, A, Q, and D; X² is an amino acid selected from S, P, Q, and N; and X³ is an amino acid selected from V, A, S, I, and F.

26. The AAV capsid protein of claim 25, wherein X¹ is L.

27. The AAV capsid protein of claim 25, wherein X¹ is G.

28. The AAV capsid protein of claim 25, wherein X² is S.

29. The AAV capsid protein of claim 25, wherein X³ is V.

30. The AAV capsid protein of claim 24, wherein the first sequence is selected from the group consisting of SNTTKSV (SEQ ID NO: 631), QNTTKSV (SEQ ID NO: 632), ADLNTTKPVMQ (SEQ ID NO: 5), AQANTTKSVDQ (SEQ ID NO: 6), AQNNTTKSVDQ (SEQ ID NO: 7), AQLNTTKNVDQ (SEQ ID NO: 8), ADLNTTKSVAQ (SEQ ID NO: 9), AQDNTTKSVFQ (SEQ ID NO: 10), ADLNTTKSVSQ (SEQ ID NO: 11), AQGNTTKSVDQ (SEQ TD NO: 12), AQGNTTKSVQQ (SEQ ID NO: 13), and AQLNTTKSVDQ. (SEQ ID NO: 14).

31. The AAV capsid protein of claim 24, comprising AAV9 as a parent AAV.

32. The AAV capsid protein of claim 31, wherein the parent AAV comprises SEQ ID NO: 1.

33. The AAV capsid protein of claim 32, comprising a 7-mer insertion inserted into the parent AAV between amino acid 588 and amino acid 589 of the parent AAV, wherein positions 587-597 of the AAV capsid protein are selected from the sequence provided in Table 1 or selected from the group consisting of SEQ ID NOs: 3-398.

34. The AAV capsid protein of claim 33, comprising one or more substitutions at positions 452-458 relative to the parent AAV, wherein positions 452-458 of the AAV capsid protein are selected from the sequence provided in Table 2 or selected from the group consisting of SEQ ID NOs: 399-630.

35. The AAV capsid protein of claim 24, wherein 60 copies of the AAV capsid protein are assembled into the AAV capsid.

36. The AAV capsid protein of claim 24, wherein the AAV capsid protein is present in VP1, VP2, and VP3 of the AAV capsid.

37. The AAV capsid protein of claim 24, further characterized by an increased transduction enrichment relative to AAV9 when measured in brain tissue in a subject when delivered to the subject systemically.

38. The AAV capsid protein of claim 24, further characterized by a decreased transduction enrichment relative to AAV9 when measured in liver tissue in a subject when delivered to the subject systemically.

39. The AAV capsid protein of claim 34, wherein the AAV capsid protein further comprises an amino acid substitution relative to the parent AAV comprising one or more of A587P, Q588D, A589M, A589D, or Q590G.
